SEQUENCE LISTING (38 sequences)

SEQ ID NO: 1 - protein, 529 aa, Listeria monocytogenes
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDPEGNEIVQHKNWSENNKSKLAHFTSSIYLPGNARNINVYAKECTGLAWEWWRTVIDDRNLPLVKNRNISIWGTTLYPKYSNKVDNPIE

SEQ ID NO: 2 - protein, 441 aa, Listeria monocytogenes
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYD

SEQ ID NO: 3 - protein, 416 aa, Listeria monocytogenes
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTD

SEQ ID NO: 4 - protein, 261 aa, Homo sapiens
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 5 - protein, 237 aa, Homo sapiens
IVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCYGVLQGITSWGSEPCALPERPSLYTKVVHYRKWIKDTIVANP

SEQ ID NO: 6 - protein, 237 aa, Homo sapiens
IVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 7 - DNA, 5873 bp, Homo sapiens
ggtgtcttaggcacactggtcttggagtgcaaaggatctaggcacgtgaggctttgtatgaagaatcggggatcgtacccaccccctgtttctgtttcatcctgggcatgtctcctctgcctttgtcccctagatgaagtctccatgagctacaagggcctggtgcatccagggtgatctagtaattgcagaacagcaagtgctagctctccctccccttccacagctctgggtgtgggagggggttgtccagcctccagcagcatggggagggccttggtcagcctctgggtgccagcagggcaggggcggagtcctggggaatgaaggttttatagggctcctgggggaggctccccagccccaagcttaccacctgcacccggagagctgtgtcaccatgtgggtcccggttgtcttcctcaccctgtccgtgacgtggattggtgagaggggccatggttggggggatgcaggagagggagccagccctgactgtcaagctgaggctctttcccccccaacccagcaccccagcccagacagggagctgggctcttttctgtctctcccagccccacttcaagcccatacccccagtcccctccatattgcaacagtcctcactcccacaccaggtccccgctccctcccacttaccccagaactttcttcccatttgcccagccagctccctgctcccagctgctttactaaaggggaagttcctgggcatctccgtgtttctctttgtggggctcaaaacctccaaggacctctctcaatgccattggttccttggaccgtatcactggtccatctcctgagcccctcaatcctatcacagtctactgacttttcccattcagctgtgagtgtccaaccctatcccagagaccttgatgcttggcctcccaatcttgccctaggatacccagatgccaaccagacacctccttctttcctagccaggctatctggcctgagacaacaaatgggtccctcagtctggcaatgggactctgagaactcctcattccctgactcttagccccagactcttcattcagtggcccacattttccttaggaaaaacatgagcatccccagccacaactgccagctctctgagtccccaaatctgcatccttttcaaaacctaaaaacaaaaagaaaaacaaataaaacaaaaccaactcagaccagaactgttttctcaacctgggacttcctaaactttccaaaaccttcctcttccagcaactgaacctcgccataaggcacttatccctggttcctagcaccccttatcccctcagaatccacaacttgtaccaagtttcccttctcccagtccaagaccccaaatcaccacaaaggacccaatccccagactcaagatatggtctgggcgctgtcttgtgtctcctaccctgatccctgggttcaactctgctcccagagcatgaagcctctccaccagcaccagccaccaacctgcaaacctagggaagattgacagaattcccagcctttcccagctccccctgcccatgtcccaggactcccagccttggttctctgcccccgtgtcttttcaaacccacatcctaaatccatctcctatccgagtcccccagttccccctgtcaaccctgattcccctgatctagcaccccctctgcaggcgctgcgcccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaagtgagtaggggcctggggtctggggagcaggtgtctgtgtcccagaggaataacagctgggcattttccccaggataacctctaaggccagccttgggactgggggagagagggaaagttctggttcaggtcacatggggaggcagggttggggctggaccaccctccccatggctgcctgggtctccatctgtgtccctctatgtctctttgtgtcgctttcattatgtctcttggtaactggcttcggttgtgtctctccgtgtgactattttgttctctctctccctctcttctctgtcttcagtctccatatctccccctctctctgtccttctctggtccctctctagccagtgtgtctcaccctgtatctctctgccaggctctgtctctcggtctctgtctcacctgtgccttctccctactgaacacacgcacgggatgggcctgggggaccctgagaaaaggaagggctttggctgggcgcggtggctcacacctgtaatcccagcactttgggaggccaaggcaggtagatcacctgaggtcaggagttcgagaccagcctggccaactggtgaaaccccatctctactaaaaatacaaaaaattagccaggcgtggtggcgcatgcctgtagtcccagctactcaggagctgagggaggagaattgcattgaacctggaggttgaggttgcagtgagccgagaccgtgccactgcactccagcctgggtgacagagtgagactccgcctcaaaaaaaaaaaaaaaaaaaaaaaaaaaaaagaaaagaaaagaaaagaaaaggaagtgttttatccctgatgtgtgtgggtatgagggtatgagagggcccctctcactccattccttctccaggacatccctccactcttgggagacacagagaagggctggttccagctggagctgggaggggcaattgagggaggaggaaggagaagggggaaggaaaacagggtatgggggaaaggaccctggggagcgaagtggaggatacaaccttgggcctgcaggcaggctacctacccacttggaaacccacgccaaagccgcatctacagctgagccactctgaggcctcccctccccggcggtccccactcagctccaaagtctctctcccttttctctcccacactttatcatcccccggattcctctctacttggttctcattcttcctttgacttcctgcttccctttctcattcatctgtttctcactttctgcctggttttgttcttctctctctctttctctggcccatgtctgtttctctatgtttctgtcttttctttctcatcctgtgtattttcggctcaccttgtttgtcactgttctcccctctgccctttcattctctctgcccttttaccctcttccttttcccttggttctctcagttctgtatctgcccttcaccctctcacactgctgtttcccaactcgttgtctgtattttggcctgaactgtgtcttcccaaccctgtgttttctcactgtttctttttctcttttggagcctcctccttgctcctctgtcccttctctctttccttatcatcctcgctcctcattcctgcgtctgcttcctccccagcaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagtgtacgcctgggccagatggtgcagccgggagcccagatgcctgggtctgagggaggaggggacaggactcctgggtctgagggaggagggccaaggaaccaggtggggtccagcccacaacagtgtttttgcctggcccgtagtcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcggtgagtcatccctactcccaagatcttgagggaaaggtgagtgggaccttaattctgggctggggtctagaagccaacaaggcgtctgcctcccctgctccccagctgtagccatgccacctccccgtgtctcatctcattccctccttccctcttctttgactccctcaaggcaataggttattcttacagcacaactcatctgttcctgcgttcagcacacggttactaggcacctgctatgcacccagcactgccctagagcctgggacatagcagtgaacagacagagagcagcccctcccttctgtagcccccaagccagtgaggggcacaggcaggaacagggaccacaacacagaaaagctggagggtgtcaggaggtgatcaggctctcggggagggagaaggggtggggagtgtgactgggaggagacatcctgcagaaggtgggagtgagcaaacacctgcgcaggggaggggagggcctgcggcacctgggggagcagagggaacagcatctggccaggcctgggaggaggggcctagagggcgtcaggagcagagaggaggttgcctggctggagtgaaggatcggggcagggtgcgagagggaacaaaggacccctcctgcagggcctcacctgggccacaggaggacactgcttttcctctgaggagtcaggaactgtggatggtgctggacagaagcaggacagggcctggctcaggtgtccagaggctgcgctggcctcctatgggatcagactgcagggagggagggcagcagggatgtggagggagtgatgatggggctgacctgggggtggctccaggcattgtccccacctgggcccttacccagcctccctcacaggctcctggccctcagtctctcccctccactccattctccacctacccacagtgggtcattctgatcaccgaactgaccatgccagccctgccgatggtcctccatggctccctagtgccctggagaggaggtgtctagtcagagagtagtcctggaaggtggcctctgtgaggagccacggggacagcatcctgcagatggtcctggcccttgtcccaccgacctgtctacaaggactgtcctcgtggaccctcccctctgcacaggagctggaccctgaagtcccttcctaccggccaggactggagcccctacccctctgttggaatccctgcccaccttcttctggaagtcggctctggagacatttctctcttcttccaaagctgggaactgctatctgttatctgcctgtccaggtctgaaagataggattgcccaggcagaaactgggactgacctatctcactctctccctgcttttacccttagggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaagtccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagttaggatggggtgtctgtgttatttgtgggatacagagatgaaagaggggtgggatcc

SEQ ID NO: 8 - protein, 238 aa, Homo sapiens
MWVPVVFLTLSVTWIGAAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSWVILITELTMPALPMVLHGSLVPWRGGV

SEQ ID NO: 9 - DNA, 1906 bp, Homo sapiens
agccccaagcttaccacctgcacccggagagctgtgtcaccatgtgggtcccggttgtcttcctcaccctgtccgtgacgtggattggtgctgcacccctcatcctgtctcggattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgtgggtcattctgatcaccgaactgaccatgccagccctgccgatggtcctccatggctccctagtgccctggagaggaggtgtctagtcagagagtagtcctggaaggtggcctctgtgaggagccacggggacagcatcctgcagatggtcctggcccttgtcccaccgacctgtctacaaggactgtcctcgtggaccctcccctctgcacaggagctggaccctgaagtcccttccccaccggccaggactggagcccctacccctctgttggaatccctgcccaccttcttctggaagtcggctctggagacatttctctcttcttccaaagctgggaactgctatctgttatctgcctgtccaggtctgaaagataggattgcccaggcagaaactgggactgacctatctcactctctccctgcttttacccttagggtgattctgggggcccacttgtctgtaatggtgtgcttcaaggtatcacgtcatggggcagtgaaccatgtgccctgcccgaaaggccttccctgtacaccaaggtggtgcattaccggaagtggatcaaggacaccatcgtggccaacccctgagcacccctatcaaccccctattgtagtaaacttggaaccttggaaatgaccaggccaagactcaagcctccccagttctactgacctttgtccttaggtgtgaggtccagggttgctaggaaaagaaatcagcagacacaggtgtagaccagagtgtttcttaaatggtgtaattttgtcctctctgtgtcctggggaatactggccatgcctggagacatatcactcaatttctctgaggacacagataggatggggtgtctgtgttatttgtggggtacagagatgaaagaggggtgggatccacactgagagagtggagagtgacatgtgctggacactgtccatgaagcactgagcagaagctggaggcacaacgcaccagacactcacagcaaggatggagctgaaaacataacccactctgtcctggaggcactgggaagcctagagaaggctgtgagccaaggagggagggtcttcctttggcatgggatggggatgaagtaaggagagggactggaccccctggaagctgattcactatggggggaggtgtattgaagtcctccagacaaccctcagatttgatgatttcctagtagaactcacagaaataaagagctgttatactgtg

SEQ ID NO: 10 - DNA, 711 bp, Artificial Sequence
  nucleotide molecule encoding KLK3 protein
attgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaacccc

SEQ ID NO: 11 - protein, 24 aa, Homo sapiens
MWVPVVFLTLSVTWIGAAPLILSR

SEQ ID NO: 12 - DNA, 2040 bp, Artificial Sequence
  sequence encoding truncated LLO fused to a PSA protein
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaacccc

SEQ ID NO: 13 - protein, 680 aa, Artificial Sequence [sequence omitted]

SEQ ID NO: 14 - DNA, 1257 bp, Artificial Sequence
  sequence encoding chimeric HER2 protein
acccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaagaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagac

SEQ ID NO: 15 - protein, 419 aa, Artificial Sequence
  chimeric HER2 protein
THLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKNIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLD

SEQ ID NO: 16 - DNA, 2586 bp, Artificial Sequence
  sequence encoding truncated LLO fused to a cHER2 protein
atgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaagaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagac

SEQ ID NO: 17 - protein, 862 aa, Artificial Sequence
  truncated LLO fused to a cHER2 protein
MKKIMLVFITLILVSLPIAQQTEAKDASAFNKENSISSMAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYDLETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKNIQEFAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLD

SEQ ID NO: 18 - protein, 390 aa, Listeria monocytogenes
MRAMMVVFITANCITINPDIIFAATDSEDSSLNTDEWEEEKTEEQPSEVNTGPRYETAREVSSRDIKELEKSNKVRNTNKADLIAMLKEKAEKGPNINNNNSEQTENAAINEEASGADRPAIQVERRHPGLPSDSAAEIKKRRKAIASSDSELESLTYPDKPTKVNKKKVAKESVADASESDLDSSMQSADESSPQPLKANQQPFFPKVFKKIKDAGKWVRDKIDENPEVKKAIVDKSAGLIDQLLTKKKSEEVNASDFPPPPTDEELRLALPETPMLLGFNAPATSEPSSFEFPPPPTDEELRLALPETPMLLGFNAPATSEPSSFEFPPPPTEDELEIIRETASSLDSSFTRGDLASLRNAINRHSQNFSDFPPIPTEEELNGRGGRP

SEQ ID NO: 19 - DNA, 1170 bp, Listeria monocytogenes
atgcgtgcgatgatggtggttttcattactgccaattgcattacgattaaccccgacataatatttgcagcgacagatagcgaagattctagtctaaacacagatgaatgggaagaagaaaaaacagaagagcaaccaagcgaggtaaatacgggaccaagatacgaaactgcacgtgaagtaagttcacgtgatattaaagaactagaaaaatcgaataaagtgagaaatacgaacaaagcagacctaatagcaatgttgaaagaaaaagcagaaaaaggtccaaatatcaataataacaacagtgaacaaactgagaatgcggctataaatgaagaggcttcaggagccgaccgaccagctatacaagtggagcgtcgtcatccaggattgccatcggatagcgcagcggaaattaaaaaaagaaggaaagccatagcatcatcggatagtgagcttgaaagccttacttatccggataaaccaacaaaagtaaataagaaaaaagtggcgaaagagtcagttgcggatgcttctgaaagtgacttagattctagcatgcagtcagcagatgagtcttcaccacaacctttaaaagcaaaccaacaaccatttttccctaaagtatttaaaaaaataaaagatgcggggaaatgggtacgtgataaaatcgacgaaaatcctgaagtaaagaaagcgattgttgataaaagtgcagggttaattgaccaattattaaccaaaaagaaaagtgaagaggtaaatgcttcggacttcccgccaccacctacggatgaagagttaagacttgctttgccagagacaccaatgcttcttggttttaatgctcctgctacatcagaaccgagctcattcgaatttccaccaccacctacggatgaagagttaagacttgctttgccagagacgccaatgcttcttggttttaatgctcctgctacatcggaaccgagctcgttcgaatttccaccgcctccaacagaagatgaactagaaatcatccgggaaacagcatcctcgctagattctagttttacaagaggggatttagctagtttgagaaatgctattaatcgccatagtcaaaatttctctgatttcccaccaatcccaacagaagaagagttgaacgggagaggcggtagacca

SEQ ID NO: 20 - protein, 226 aa, Artificial Sequence
  truncated ActA fused to hly signal peptide
MKKIMLVFITLILVSLPIAQQTEASRATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDK

SEQ ID NO: 21 - DNA, 678 bp, Artificial Sequence
  sequence encoding truncated ActA fused to hly signal peptide
atgaaaaaaataatgctagtttttattacacttatattagttagtctaccaattgcgcaacaaactgaagcatctagagcgacagatagcgaagattccagtctaaacacagatgaatgggaagaagaaaaaacagaagagcagccaagcgaggtaaatacgggaccaagatacgaaactgcacgtgaagtaagttcacgtgatattgaggaactagaaaaatcgaataaagtgaaaaatacgaacaaagcagacctaatagcaatgttgaaagcaaaagcagagaaaggtccgaataacaataataacaacggtgagcaaacaggaaatgtggctataaatgaagaggcttcaggagtcgaccgaccaactctgcaagtggagcgtcgtcatccaggtctgtcatcggatagcgcagcggaaattaaaaaaagaagaaaagccatagcgtcgtcggatagtgagcttgaaagccttacttatccagataaaccaacaaaagcaaataagagaaaagtggcgaaagagtcagttgtggatgcttctgaaagtgacttagattctagcatgcagtcagcagacgagtctacaccacaacctttaaaagcaaatcaaaaaccatttttccctaaagtatttaaaaaaataaaagatgcggggaaatgggtacgtgataaa

SEQ ID NO: 22 - protein, 14 aa, Listeria monocytogenes
KTEEQPSEVNTGPR

SEQ ID NO: 23 - protein, 28 aa, Listeria monocytogenes
KASVTDTSEGDLDSSMQSADESTPQPLK

SEQ ID NO: 24 - protein, 20 aa, Listeria monocytogenes
KNEEVNASDFPPPPTDEELR

SEQ ID NO: 25 - protein, 33 aa, Listeria monocytogenes
RGGIPTSEEFSSLNSGDFTDDENSETTEEEIDR

SEQ ID NO: 26 - protein, 17 aa, Streptococcus pyogenes
KQNTASTETTTTNEQPK

SEQ ID NO: 27 - protein, 17 aa, Streptococcus equisimilis
KQNTANTETTTTNEQPK

SEQ ID NO: 28 - DNA, 6523 bp, Artificial Sequence
  plasmid pAdv142
cggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaacccctaacccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcgcggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaattaa

SEQ ID NO: 29 - DNA, 36 bp, Artificial Sequence
  primer
cggaattcggatccgcgccaaatcattggttgattg

SEQ ID NO: 30 - DNA, 37 bp, Artificial Sequence
  primer
gcgagtcgacgtcggggttaatcgtaatgcaattggc

SEQ ID NO: 31 - DNA, 35 bp, Artificial Sequence
  primer
gcgagtcgacccatacgacgttaattcttgcaatg

SEQ ID NO: 32 - DNA, 39 bp, Artificial Sequence
  primer
gatactgcagggatccttcccttctcggtaatcagtcac

SEQ ID NO: 33 - DNA, 19 bp, Artificial Sequence
  primer
tgggatggccaagaaattc

SEQ ID NO: 34 - DNA, 22 bp, Artificial Sequence
  primer
ctaccatgtcttccgttgcttg

SEQ ID NO: 35 - DNA, 7075 bp, Artificial Sequence
  pAdv164 sequence
cggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaagaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagactaatctagacccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcgcggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaattaa

SEQ ID NO: 36 - protein, 9 aa, Artificial Sequence
  mapped HLA-A2 restricted extracellular epitope
HLYQGCQVV

SEQ ID NO: 37 - protein, 9 aa, Artificial Sequence
  mapped HLA-A2 restricted extracellular epitope
KIFGSLAFL

SEQ ID NO: 38 - protein, 9 aa, Artificial Sequence
  mapped HLA-A2 restricted intracellular epitope
RLLQETELV

(19) United States
(12) Patent Application Publication
(10) Pub. No.: US 2018/0325964 A1
(43) Pub. Date: Nov. 15, 2018

(54) MANUFACTURING DEVICE AND METHOD OF AN IMMUNOTHERAPEUTIC FORMULATION COMPRISING A RECOMBINANT LISTERIA STRAIN

(71) Applicant: Advaxis, Inc., Princeton, NJ (US)

(72) Inventors: Anil Eapen, Princeton, NJ (US); Robert Petit, Newtown (Wrightstown), PA (US)

(21) Appl. No.: 15/777,480

(22) PCT Filed: Nov. 18, 2016
(86) PCT No.: PCT/IB2016/056980

§ 371 Date: May 18, 2018

Related U.S. Application Data
(60) Provisional application No. 62/258,301, filed on Nov. 20, 2015.
(60) Provisional application No. 62/342,037, filed on May 26, 2016.

(51) Int. Cl. A61K 35/74; C12N 9/64; B01D 61/18; B01D 61/22; A61P 35/00; A61K 39/00

(52) U.S. Cl. CPC A61K 35/74; C12N 9/6445; B01D 61/18; B01D 61/22; A61P 35/00; A61K 39/001194; C12Y 206/01021; A61K 2039/523; A61K 2039/522; B01D 2315/10; C12Y 207/10001; C12Y 304/21077; B01D 2315/16

(57) ABSTRACT

Provided herein are an apparatus and process for manufacturing a formulation comprising a drug substance, said drug substance comprising a recombinant Listeria strain comprising a prostate specific antigen (PSA) or a chimeric HER2 antigen fused to a Listeriolysin O (LLO) protein fragment.

### FIELD OF INVENTION

The present disclosure discloses a process for manufacturing a formulation comprising a drug substance, said drug substance comprising a recombinant Listeria strain comprising a prostate specific antigen or a chimeric HER2 antigen fused to a Listeriolysin O (LLO) protein fragment.

### BACKGROUND

Listeria monocytogenes (Lm) is an intracellular pathogen that primarily infects antigen presenting cells and has adapted for life in the cytoplasm of these cells. Host cells, such as macrophages, actively phagocytose L. monocytogenes and the majority of the bacteria are degraded in the phagolysosome. Some of the bacteria escape into the host cytosol by perforating the phagosomal membrane through the action of a hemolysin, listeriolysin O (LLO). Once in the cytosol, L. monocytogenes can polymerize the host actin and pass directly from cell to cell further evading the host immune system and resulting in a negligible antibody response to L. monocytogenes.

Her-2/neu is a 185 kDa glycoprotein that is a member of the epidermal growth factor receptor (EGFR) family of tyrosine kinases, and consists of an extracellular domain, a transmembrane domain, and an intracellular domain which is known to be involved in cellular signaling. In humans, the HER2 antigen is overexpressed in 25 to 40% of all breast cancers and is also overexpressed in many cancers of the ovaries, lung, pancreas, bones, brain, and gastrointestinal tract. The overexpression of Her-2 is associated with uncontrolled cell growth and signaling, both of which contribute to the development of tumors. Patients with cancers that overexpress Her-2 exhibit tolerance even with detectable humoral, CD8⁺ T cell, and CD4⁺ T cell responses directed against Her-2.

The Her2/neu is too big to fit in Lm which necessitated the generation of Her2/neu fragments. Having found activity in each fragment independently the present disclosure incorporates all of the active sites from each of the independent fragments. Thus, a immunotherapy based upon a chimeric protein made by fusing of two of the extracellular and one intracellular fragments of the protein which included most of the known MHC class I epitopes of the Her2/neu receptor (Lm-LLO-ChHer2) has been generated. All of these immunotherapies were shown to be immunogenic and efficacious in regressing pre-established tumors in FVB/N mice and delay the onset of spontaneous mammary tumors in Her2/neu-expressing transgenic animals. The encouraging results from these preliminary experiments suggested that a recombinant Listeria-Her2/neu immunotherapy could be generated which could break the tolerance toward the Her2/neu self-antigen. However, the Listeria-Her2/neu immunotherapies developed thus far have been based on an attenuated Listeria platform which used the antibiotic marker (cat), for in vitro selection of the recombinant bacteria in the presence of chloramphenicol. For clinical use, not only high attenuation is important, but also the absence of resistance to antibiotics.

Prostate-specific antigen (PSA), also known as kallikrein III (KLK3), seminin, semenogelase, γ-serinoprotein and P-30 antigen, is a 34-kD glycoprotein produced almost exclusively by the prostate gland. PSA is produced for the ejaculate, where it liquefies semen in the seminal coagulum and allows sperm to swim freely. It is also believed to be instrumental in dissolving cervical mucus, allowing the entry of sperm into the uterus.

PSA is present in small quantities in the serum of men with healthy prostates, but is often elevated in the presence of prostate cancer or other prostate disorders. Increased levels of PSA may suggest the presence of prostate cancer. The PSA rate of rise may have value in prostate cancer prognosis. Men with prostate cancer whose PSA level increased by more than 2.0 ng per milliliter during the year before the diagnosis of prostate cancer have a higher risk of death from prostate cancer despite undergoing radical prostatectomy. PSA also is found in the serum of women with breast, lung, or uterine cancer and in some patients with renal cancer.

In addition, PSA is not a unique indicator of prostate cancer, but may also detect prostatitis or benign prostatic hyperplasia. 30 percent of patients with high PSA have prostate cancer diagnosed after biopsy. Prostate cancer is the most frequent type of cancer in American men and it is the second cause of cancer related death in this population. Prostate Specific Antigen (PSA) is a marker for prostate cancer that is highly expressed by prostate tumors.

Tumor evasion of the host immune response via escape mutations has been well documented and remains a major obstacle in tumor therapy. Thus, there is a need for developing a immunotherapy that has high therapeutic efficacy and that does not result in escape mutations. The subject matter of the present disclosure meets this need by providing a manufacturing process for a recombinant Listeria-Her2/neu immunotherapy (ADXS31-164) and a recombinant Listeria-PSA (ADXS31-142) immunotherapy that were generated using the LmddA immunotherapy vector which has a well-defined attenuation mechanism and is devoid of antibiotic selection markers.

### SUMMARY OF THE INVENTION

In one aspect, some embodiments of the disclosure relate to a process for the manufacturing of a formulation comprising a drug substance, said drug substance comprising a recombinant Listeria strain, said recombinant Listeria strain comprising a nucleic acid comprising an open reading frame encoding a recombinant polypeptide, said recombinant polypeptide comprising a prostate specific antigen (PSA) or a chimeric HER2 (cHER2) antigen fused to a Listeriolysin O (LLO) polypeptide, the method comprising the steps of:
a) Aseptically preparing a first pre-culture media (PC1) in a container and a second pre-culture media (PC2) in at least two containers.
   i. Wherein said PC1 and PC2 is incubated for 12-24 h to ensure sterility.
b) Aseptically adding a working cell bank (WCB) comprising said recombinant Listeria into PC1.
   i. Wherein said PC1 is incubated until a target optical density (OD) is reached.
c) Aseptically inoculating each container of PC2 with an aliquot from said PC1.
d) Incubating each container in c) until a target optical density (OD) is reached and pooling culture media from each of said container into a larger biotainer.
e) Preparing fermentation media and adding said fermentation media into a fermenter system. In another aspect, the fermentation media is pre-incubated for 12±6 h before inoculating in order to verify sterility.
f) Inoculating said fermentation media with the pooled culture media from d) and initiating a fermentation process until a target optical density (OD) is reached.
g) Aseptically connecting said fermenter system to a filtration system and concentrating said drug substance within said fermentation media to a desired weight.
h) Obtaining a retentate or harvest solution comprising said drug substance from step g) and exchanging the spent fermentation media with an appropriate formulation for human use.
i) Aseptically transferring said harvest comprising said drug substance into biotainers.
   i. Wherein said biotainers are stored at −80° C.±10° C. until they are aseptically filled into vials for clinical use.
   ii. 2-7 days prior to the filling process, the viable cell count of one drug substance aliquot is determined for the calculation of the dilution factor and required amount for formulation of the drug substance with the same buffer used for the diafiltration step in step h).
   iii. The required number of drug substance biotainers are thawed at 5±3° C. for ≤16 hours.
   iv. The drug substance is formulated under aseptic conditions and aseptically filled into vials.
j) Disinfecting, inspecting, labeling, packaging and distributing vials to clinical sites.

In another related aspect, disclosed herein are methods of treating, protecting against, and inducing an immune response against a tumor or cancer, comprising the step of administering to a subject a recombinant Listeria strain, comprising a fusion peptide that comprises an LLO fragment and an PSA or a chimeric HER2 (cHER2) antigen. The present disclosure also provides methods for inducing an anti-PSA or anti-HER2 CTL response in a human subject and treating PSA- or cHER2-mediated diseases, disorders, and symptoms, comprising administration of the recombinant Listeria strain.

In another related aspect, the disclosure relates to a tangential flow filtration (TFF) device comprising of a concentration section and a diafiltration section for concentrating and diafiltrating a drug product comprising a recombinant Listeria strain, wherein said comprising a retentate container 1, operably linked via flow fluid conduits 5 to a permeate container 2.

Other features and advantages of the subject matter disclosed herein will become apparent from the following detailed description examples and figures. It should be understood, however, that the detailed description and the specific examples while indicating embodiments of the disclosure are given by way of illustration only, since various changes and modifications within the spirit and scope of the disclosure will become apparent to those skilled in the art from this detailed description.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1A-B. (FIG. 1A) Schematic representation of the chromosomal region of the Lmdd-143 and LmddA-143 after klk3 integration and actA deletion; (FIG. 1B) The klk3 gene is integrated into the Lmdd and LmddA chromosome. PCR from chromosomal DNA preparation from each construct using klk3 specific primers amplifies a band of 714 bp corresponding to the klk3 gene, lacking the secretion signal sequence of the wild type protein.

FIGS. 2A-D. (FIG. 2A) Map of the pADV134 plasmid. (FIG. 2B) Proteins from LmddA-134 culture supernatant were precipitated, separated in a SDS-PAGE, and the LLO-E7 protein detected by Western-blot using an anti-E7 monoclonal antibody. The antigen expression cassette consists of hly promoter, ORF for truncated LLO and human PSA gene (klk3). (FIG. 11C) Map of the pADV142 plasmid. (FIG. 2D) Western blot showed the expression of LLO-PSA fusion protein using anti-PSA and anti-LLO antibody.

FIGS. 3A-B. (FIG. 3A) Plasmid stability in vitro of LmddA-LLO-PSA if cultured with and without selection pressure (D-alanine). Strain and culture conditions are listed first and plates used for CFU determination are listed after. (FIG. 3B) Clearance of LmddA-LLO-PSA in vivo and assessment of potential plasmid loss during this time. Bacteria were injected i.v. and isolated from spleen at the time point indicated. CFUs were determined on BHI and BHI+D-alanine plates.

FIGS. 4A-B. (FIG. 4A) In vivo clearance of the strain LmddA-LLO-PSA after administration of 10⁸ CFU in C57BL/6 mice. The number of CFU were determined by plating on BHI/str plates. The limit of detection of this method was 100 CFU. (FIG. 4B) Cell infection assay of J774 cells with 10403S, LmddA-LLO-PSA and XFL7 strains.

FIGS. 5A-E. (FIG. 5A) PSA tetramer-specific cells in the splenocytes of naïve and LmddA-LLO-PSA immunized mice on day 6 after the booster dose. (FIG. 5B) Intracellular cytokine staining for IFN-γ in the splenocytes of naïve and LmddA-LLO-PSA immunized mice were stimulated with PSA peptide for 5 h. Specific lysis of EL4 cells pulsed with PSA peptide with in vitro stimulated effector T cells from LmddA-LLO-PSA immunized mice and naïve mice at different effector/target ratio using a caspase based assay (FIG. 5C) and a europium based assay (FIG. 5D). Number of IFNγ spots in naïve and immunized splenocytes obtained after stimulation for 24 h in the presence of PSA peptide or no peptide (FIG. 5E).

FIGS. 6A-C. Immunization with LmddA-142 induces regression of Tramp-C1-PSA (TPSA) tumors. Mice were left untreated (n=8) (FIG. 6A) or immunized i.p. with LmddA-142 (1×10⁸ CFU/mouse) (n=8) (FIG. 6B) or Lm-LLO-PSA (n=8), (FIG. 6C) on days 7, 14 and 21. Tumor sizes were measured for each individual tumor and the values expressed as the mean diameter in millimeters. Each line represents an individual mouse.

FIGS. 7A-B. (FIG. 7A) Analysis of PSA-tetramer⁺CD8⁺ T cells in the spleens and infiltrating T-PSA-23 tumors of untreated mice and mice immunized with either an Lm control strain or LmddA-LLO-PSA (LmddA-142). (FIG. 7B) Analysis of CD4⁺ regulatory T cells, which were defined as CD25⁺FoxP3⁺, in the spleens and infiltrating T-PSA-23 tumors of untreated mice and mice immunized with either an Lm control strain or LmddA-LLO-PSA.

FIGS. 8A-B. (FIG. 8A) Schematic representation of the chromosomal region of the Lmdd-143 and LmddA-143 after klk3 integration and actA deletion; (FIG. 8B) The klk3 gene is integrated into the Lmdd and LmddA chromosome. PCR from chromosomal DNA preparation from each construct using klk3 specific primers amplifies a band of 760 bp corresponding to the klk3 gene.

FIGS. 9A-C. (FIG. 9A) Lmdd-143 and LmddA-143 secretes the LLO-PSA protein. Proteins from bacterial culture supernatants were precipitated, separated in a SDS-PAGE and LLO and LLO-PSA proteins detected by Western-blot using an anti-LLO and anti-PSA antibodies; (FIG. 9B) LLO produced by Lmdd-143 and LmddA-143 retains hemolytic activity. Sheep red blood cells were incubated with serial dilutions of bacterial culture supernatants and hemolytic activity measured by absorbance at 590 nm; (FIG. 9C) Lmdd-143 and LmddA-143 grow inside the macrophage-like J774 cells. J774 cells were incubated with bacteria for 1 hour followed by gentamicin treatment to kill extracellular bacteria. Intracellular growth was measured by plating serial dilutions of J774 lysates obtained at the indicated timepoints. Lm 10403S was used as a control in these experiments.

FIG. 10. Immunization of mice with Lmdd-143 and LmddA-143 induces a PSA-specific immune response. C57BL/6 mice were immunized twice at 1-week interval with 1×10⁸ CFU of Lmdd-143, LmddA-143 or LmddA-142 and 7 days later spleens were harvested. Splenocytes were stimulated for 5 hours in the presence of monensin with 1 μM of the PSA₆₅₋₇₄ peptide. Cells were stained for CD8, CD3, CD62 L and intracellular IFN-γ and analyzed in a FACS Calibur cytometer.

FIGS. 11A-B. Construction of ADXS31-164. (FIG. 11A) Plasmid map of pAdv164, which harbors bacillus subtilis dal gene under the control of constitutive Listeria p60 promoter for complementation of the chromosomal dal-dat deletion in LmddA strain. It also contains the fusion of truncated LLO₍₁₋₄₄₁₎ to the chimeric human Her2/neu gene, which was constructed by the direct fusion of 3 fragments the Her2/neu: EC1 (aa 40-170), EC2 (aa 359-518) and ICI (aa 679-808). (FIG. 11B) Expression and secretion of tLLO-ChHer2 was detected in Lm-LLO-ChHer2 (Lm-LLO-138) and LmddA-LLO-ChHer2 (ADXS31-164) by western blot analysis of the TCA precipitated cell culture supernatants blotted with anti-LLO antibody. A differential band of ˜104 KD corresponds to tLLO-ChHer2. The endogenous LLO is detected as a 58 KD band. Listeria control lacked ChHer2 expression.

FIGS. 12A-C. Immunogenic properties of ADXS31-164 (FIG. 12A) Cytotoxic T cell responses elicited by Her2/neu Listeria-based immunotherapies in splenocytes from immunized mice were tested using NT-2 cells as stimulators and 3T3/neu cells as targets. Lm-control was based on the LmddA background that was identical in all ways but expressed an irrelevant antigen (HPV16-E7). (FIG. 12B) IFN-γ secreted by the splenocytes from immunized FVB/N mice into the cell culture medium, measured by ELISA, after 24 hours of in vitro stimulation with mitomycin C treated NT-2 cells. (FIG. 12C) IFN-γ secretion by splenocytes from HLA-A2 transgenic mice immunized with the chimeric immunotherapy, in response to in vitro incubation with peptides from different regions of the protein. A recombinant ChHer2 protein was used as positive control and an irrelevant peptide or no peptide groups constituted the negative controls as listed in the figure legend. IFN-γ secretion was detected by an ELISA assay using cell culture supernatants harvested after 72 hours of co-incubation. Each data point was an average of triplicate data+/−standard error. * P value<0.001.

FIG. 13. Tumor Prevention Studies for Listeria-ChHer2/neu immunotherapies Her2/neu transgenic mice were injected six times with each recombinant Listeria-ChHer2 or a control Listeria immunotherapy. Immunizations started at 6 weeks of age and continued every three weeks until week 21. Appearance of tumors was monitored on a weekly basis and expressed as percentage of tumor free mice. *p<0.05, N=9 per group.

FIG. 14. Effect of immunization with ADXS31-164 on the % of Tregs in Spleens. FVB/N mice were inoculated s.c. with 1×10⁶ NT-2 cells and immunized three times with each immunotherapy at one week intervals. Spleens were harvested 7 days after the second immunization. After isolation of the immune cells, they were stained for detection of Tregs by anti CD3, CD4, CD25 and FoxP3 antibodies. Dot-plots of the Tregs from a representative experiment showing the frequency of CD25⁺/FoxP3⁺ T cells, expressed as percentages of the total CD3⁺ or CD3⁺CD4⁺ T cells across the different treatment groups.

FIGS. 15A-B. Effect of immunization with ADXS31-164 on the % of tumor infiltrating Tregs in NT-2 tumors. FVB/N mice were inoculated s.c. with 1×10⁶ NT-2 cells and immunized three times with each immunotherapy at one week intervals. Tumors were harvested 7 days after the second immunization. After isolation of the immune cells, they were stained for detection of Tregs by anti CD3, CD4, CD25 and FoxP3 antibodies. (FIG. 15A). Dot-plots of the Tregs from a representative experiment (FIG. 15B). Frequency of CD25⁺/FoxP3⁺ T cells, expressed as percentages of the total CD3⁺ or CD3⁺CD4⁺ T cells (left panel) and intratumoral CD8/Tregs ratio (right panel) across the different treatment groups. Data is shown as mean±SEM obtained from 2 independent experiments.

FIGS. 16A-C. Vaccination with ADXS31-164 can delay the growth of a breast cancer cell line in the brain. Balb/c mice were immunized thrice with ADXS31-164 or a control Listeria immunotherapy. EMT6-Luc cells (5,000) were injected intracranially in anesthetized mice. (FIG. 16A) Ex vivo imaging of the mice was performed on the indicated days using a Xenogen X-100 CCD camera. (FIG. 16B) Pixel intensity was graphed as number of photons per second per cm2 of surface area; this is shown as average radiance. (FIG. 16C) Expression of Her2/neu by EMT6-Luc cells, 4T1-Luc and NT-2 cell lines was detected by Western blots, using an anti-Her2/neu antibody. J774.A2 cells, a murine macrophage like cell line was used as a negative control.

FIG. 17. Flow diagram of manufacturing process of drug substance (ADXS31-142 and ADXS31-164).

FIG. 18. Shows a process for preparing fermentation media.

FIG. 19. Shows a process for preparing IM Sodium Hydroxide (NaOH) solution.

FIG. 20. Shows a process for preparing a washing buffer.

FIG. 21. Shows a process for preparing inoculum bag(s).

FIG. 22. Shows a process for carrying out fermentation of the Listeria construct disclosed herein.

FIG. 23. Shows a process for setting up and carrying out tangential flow filtration and fill.

FIGS. 24A-24C. Show Tangential Flow Filtration (TFF) manifolds according to some embodiments discussed herein. FIG. 51A shows a TFF manifold and FIG. 51B shows the descriptions of several parts of the TFF manifold. FIG. 51C shows another TFF manifold according to some embodiments discussed herein.

FIG. 25. Shows an example fill manifold that may connect to the TFF manifolds.

FIG. 26. Shows a fill manifold used for collecting the final product in one or more bags.

FIG. 27. Shows the legends for the labels in FIG. 25A through FIG. 27.

FIG. 28. Shows a table comparing Reynolds number, pump flow rate, fiber count, velocity, kinematic viscosity, flow/fiber, unit length, internal diameter, fiber volume, transit time, and characteristic length for several example embodiments.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION

In one embodiment, disclosed herein is a manufacturing device and process for the manufacturing of a formulation comprising a drug substance (DS), said drug substance comprising a recombinant Listeria strain, said recombinant Listeria strain comprising a nucleic acid comprising an open reading frame encoding a recombinant polypeptide, said recombinant polypeptide comprising a prostate specific antigen (PSA) or a chimeric HER2 (cHER2) antigen fused to a Listeriolysin O (LLO) polypeptide, the method comprising the steps of:
a) Aseptically preparing a first pre-culture media (PC1) in a container and a second pre-culture media (PC2) in at least two containers.
   i. Wherein said PC1 and PC2 is incubated for 12-24 h to ensure sterility.
b) Aseptically adding a working cell bank (WCB) comprising said recombinant Listeria into PC1.
   i. Wherein said PC1 is incubated until a target optical density (OD) is reached.
c) Aseptically inoculating each container of PC2 with an aliquot from said PC1.
d) Incubating each container in c) until a target optical density (OD) is reached and pooling culture media from each of said container into a larger biotainer.
e) Preparing fermentation media and adding said fermentation media into a fermenter system. In another embodiment, the fermentation media is pre-incubated for 12±6 h before inoculating in order to verify sterility.
f) Inoculating said fermentation media with the pooled culture media from d) and initiating a fermentation process until a target optical density (OD) is reached.
g) Aseptically connecting said fermenter system to a filtration system and concentrating said drug substance within said fermentation media to a desired weight.
h) Obtaining a retentate or harvest solution comprising said drug substance from step g) and exchanging the spent fermentation media with an appropriate formulation for human use.
i) Aseptically transferring said harvest comprising said drug substance into biotainers.
j) Disinfecting, inspecting, labeling, packaging and distributing vials to clinical sites.

In another embodiment, said PC1 and PC2 are aseptically sampled and tested for Optical Density (OD_{600 nm}), and pH at regular intervals until said target OD is reached.

In another embodiment, the pooled culture from d) is sampled to determine the viable cell count (VCC), OD, and pH.

In one embodiment, said initiation of said fermentation process is preceded by a pre-incubation step of the fermentation media. In another embodiment, said pre-incubation step comprises regulating and maintaining a constant temperature, constant pH, and constant dissolved oxygen percentage (pO2). In another embodiment, said pO2 level is controlled by sparger aeration with oxygen. In another embodiment, said pH of said fermentation process is controlled using an alkylating agent.

In one embodiment, a fermentation process disclosed herein is stopped by cooling the fermentation media to a temperature of ≤20° C. after said target OD has been reached. In another embodiment, a fermentation process is monitored using pO2 and is stopped when a target pO2 level is reached. In another embodiment, a fermentation process is monitored by measuring the pH and is stopped when a target pH is reached.

In one embodiment, a fermented media is prepared according to the steps disclosed herein (see Example 14). In another embodiment, a fermented media disclosed herein is aseptically sampled and tested for OD, pH and viable cell count (VCC) prior to connecting to a filtration system. In another embodiment, a fermented media disclosed herein is aseptically sampled and tested for OD, pH and viable cell count (VCC) prior to connecting to a cross flow filtration system/tangential flow filtration system disclosed herein.

In one embodiment, one or more biotainers disclosed herein are stored at −80° C.±10° C. until they are aseptically filled/aliquoted into vials for clinical use. It will be appreciated by a skilled artisan that other types of containers other than a biotainer may be used in the manufacturing process disclosed herein. Such containers may include but are not limited to flasks, including Erlenmeyer flasks, bottles and the like.

In another embodiment, 2-7 days prior to the filling process, the viable cell count (VCC) of one drug substance aliquot is determined for the calculation of the dilution factor and required amount for formulation of the drug substance with the same buffer used for the diafiltration step in step h). It will be appreciated by a skilled artisan that the above-mentioned range of days make be varied or adjusted as desired in order to optimize a manufacturing process disclosed herein. Such variations include but are not limited to broader ranges such as 1-10 days, 1-15 days, or 1-20 days, or narrower ranges such as 2-4 days, 2-5 days, or 2-6 days.

In another embodiment, the required number of drug substance biotainers are thawed at 5±3° C. for about ≤16 hours. In another embodiment, the required number of drug substance biotainers are thawed at about 5±3° C. for about 1-5, 5-10, or 10-16 hours. It will be appreciated by a skilled artisan that thawing temperatures are not strictly restricted to the above mentioned range but may vary based on other variables, including, but not limited to, atmospheric or artificial air pressure.

In one embodiment, a drug substance disclosed herein is formulated under aseptic conditions and is aseptically filled or aliquoted into vials.

In one embodiment, a drug substance disclosed herein comprises a recombinant Listeria also disclosed herein. In another embodiment, a drug substance disclosed is a recombinant Listeria also disclosed herein. In one embodiment, the recombinant Listeria strain of disclosed herein is a recombinant Listeria monocytogenes strain. In another embodiment, the Listeria strain is a recombinant Listeria seeligeri strain. In another embodiment, the Listeria strain is a recombinant Listeria grayi strain. In another embodiment, the Listeria strain is a recombinant Listeria ivanovii strain. In another embodiment, the Listeria strain is a recombinant Listeria murrayi strain. In another embodiment, the Listeria strain is a recombinant Listeria welshimeri strain. In another embodiment, the Listeria strain is a recombinant strain of any other Listeria species known in the art.

In another embodiment, the recombinant Listeria disclosed herein comprises a nucleic acid molecule in a plasmid in said recombinant Listeria. In another embodiment, said plasmid is stably maintained in said recombinant Listeria. In another embodiment, said plasmid lacks antibiotic resistance genes. In another embodiment, said plasmid does not confer antibiotic resistance upon said recombinant Listeria. In another embodiment, said plasmid is an integrative plasmid. In another embodiment, said plasmid is an extrachromosomal or episomal plasmid.

In one embodiment, a recombinant polypeptide disclosed herein is expressed by a recombinant Listeria.

In another embodiment, a recombinant Listeria strain of the present disclosure has been passaged through an animal host. In another embodiment, the passaging maximizes efficacy of the strain as an immunotherapy vector. In another embodiment, the passaging stabilizes the immunogenicity of the Listeria strain. In another embodiment, the passaging stabilizes the virulence of the Listeria strain. In another embodiment, the passaging increases the immunogenicity of the Listeria strain. In another embodiment, the passaging increases the virulence of the Listeria strain. In another embodiment, the passaging removes unstable sub-strains of the Listeria strain. In another embodiment, the passaging reduces the prevalence of unstable sub-strains of the Listeria strain. In another embodiment, the Listeria strain contains a genomic insertion of the gene encoding the antigen-containing recombinant peptide. In another embodiment, the Listeria strain carries a plasmid comprising the gene encoding the antigen-containing recombinant peptide. In another embodiment, the passaging is performed by any other method known in the art.

In another embodiment, the recombinant Listeria strain utilized in methods of the present disclosure has been stored in a frozen cell bank prior to adding into a fermenter disclosed herein. In another embodiment, the recombinant Listeria strain has been stored in a lyophilized cell bank prior to adding into a fermenter disclosed herein.

In another embodiment, the cell bank of methods and compositions of the present disclosure is a master cell bank (MCB). In another embodiment, the cell bank is a working cell bank (WCB). In another embodiment, the cell bank is Good Manufacturing Practice (GMP) cell bank. In another embodiment, the cell bank is intended for production of clinical-grade material. In another embodiment, the cell bank conforms to regulatory practices for human use. In another embodiment, the cell bank is any other type of cell bank known in the art.

In one embodiment, 2 mL cryovials containing 1 mL of the ADXS31-142 or ADXS31-164 Working Cell Bank (WCB) are thawed prior to adding a drug substance disclosed herein into the fermenter system disclosed herein. In another embodiment, 2-5 mL cryovials containing the 1-5 mL of the ADXS31-142 or ADXS31-164 Working Cell Bank (WCB) are thawed prior to adding a drug substance disclosed herein into the fermenter system disclosed herein. In another embodiment, 1-5 ml of the ADXS31-142 or ADXS31-164 Working Cell Bank (WCB) are present in the cryovials. In another embodiment, 5-10 ml of the ADXS31-142 or ADXS31-164 Working Cell Bank (WCB) are present in the cryovials. In another embodiment, a cryovial disclosed herein is a polypropylene cryovial, however, it will be understood by a skilled artisan that other suitable cryovials known in the art may be used.

“Good Manufacturing Practices” are defined, in another embodiment, by (21 CFR 210-211) of the United States Code of Federal Regulations. In another embodiment, “Good Manufacturing Practices” are defined by other standards for production of clinical-grade material or for human consumption; e.g. standards of a country other than the United States.

In another embodiment, a recombinant Listeria strain utilized in methods of the present disclosure is from a batch of immunotherapy doses.

In another embodiment, a recombinant Listeria strain utilized in methods of the present disclosure is from a frozen stock produced by the process disclosed herein.

In another embodiment, a recombinant Listeria strain utilized in methods of the present disclosure is from a lyophilized stock produced by the process disclosed herein.

In another embodiment, a cell bank, frozen stock, or batch of immunotherapy doses of the present disclosure exhibits viability upon thawing of greater than 90%. In another embodiment, the thawing follows storage for cryopreservation or frozen storage for 2 hours. In another embodiment, the thawing follows storage for cryopreservation or frozen storage for 6 hours. In another embodiment, the thawing follows storage for cryopreservation or frozen storage for 12 hours. In another embodiment, the thawing follows storage for cryopreservation or frozen storage for 24 hours. In another embodiment, the storage is for 2 days. In another embodiment, the storage is for 3 days. In another embodiment, the storage is for 4 days. In another embodiment, the storage is for 1 week. In another embodiment, the storage is for 2 weeks. In another embodiment, the storage is for 3 weeks. In another embodiment, the storage is for 1 month. In another embodiment, the storage is for 2 months. In another embodiment, the storage is for 3 months. In another embodiment, the storage is for 5 months. In another embodiment, the storage is for 6 months. In another embodiment, the storage is for 9 months. In another embodiment, the storage is for 1 year.

In another embodiment, a cell bank, frozen stock, or batch of immunotherapy doses of the present disclosure is cryopreserved by a method that comprises growing a culture of the Listeria strain in a nutrient media, freezing the culture in a solution comprising an antifreeze agent, and storing the Listeria strain at below −20 degrees Celsius. In another embodiment, the antifreeze agent is propylene glycol. In another embodiment, the antifreeze agent is ethylene glycol. In another embodiment, the antifreeze agent is glycerol. In another embodiment, the antifreeze agent is sucrose. In another embodiment, the temperature is about −70 degrees Celsius. In another embodiment, the temperature is about −70-−80 degrees Celsius.

In another embodiment, a cell bank, frozen stock, or batch of immunotherapy doses of the present disclosure is cryopreserved by a method that comprises growing a culture of the Listeria strain in a defined media, freezing the culture in a solution comprising an antifreeze agent, and storing the Listeria strain at below −20 degrees Celsius. In another embodiment, the antifreeze agent is propylene glycol. In another embodiment, the antifreeze agent is ethylene glycol. In another embodiment, the antifreeze agent is glycerol. In another embodiment, the antifreeze agent is sucrose. In another embodiment, the temperature is about −70 degrees Celsius. In another embodiment, the temperature is about −70-−80 degrees Celsius. In another embodiment, any defined microbiological media of the present disclosure may be used in this method.

In another embodiment of methods and compositions disclosed herein, the culture (e.g. the culture of a Listeria immunotherapy strain that is used to produce a batch of Listeria immunotherapy doses) is inoculated from a cell bank. In another embodiment, the culture is inoculated from a frozen stock. In another embodiment, the culture is inoculated from a starter culture. In another embodiment, the culture is inoculated from a colony. In another embodiment, the culture is inoculated at mid-log growth phase. In another embodiment, the culture is inoculated at approximately mid-log growth phase. In another embodiment, the culture is inoculated at another growth phase. In another embodiment, the WCB is removed from ≤−70° C. storage and thawed at room temperature prior to adding into a fermenter system.

In another embodiment of methods and compositions disclosed herein, the solution used for freezing contains ethylene glycol, propylene glycol, glycerol or sucrose in an amount of 1-20%. In another embodiment, the amount is 2%. In another embodiment, the amount is 20%. In another embodiment, the amount is 1%. In another embodiment, the amount is 1.5%. In another embodiment, the amount is 3%. In another embodiment, the amount is 4%. In another embodiment, the amount is 5%. In another embodiment, the amount is 2%. In another embodiment, the amount is 2%. In another embodiment, the amount is 7%. In another embodiment, the amount is 9%. In another embodiment, the amount is 10%. In another embodiment, the amount is 12%. In another embodiment, the amount is 14%. In another embodiment, the amount is 16%. In another embodiment, the amount is 18%. In another embodiment, the amount is 222%. In another embodiment, the amount is 25%. In another embodiment, the amount is 30%. In another embodiment, the amount is 35%. In another embodiment, the amount is 40%.

In another embodiment, the solution used for freezing contains another colligative additive or additive with anti-freeze properties, in place of glycerol. In another embodiment, the solution used for freezing contains another colligative additive or additive with anti-freeze properties, in addition to glycerol. In another embodiment, the additive is mannitol. In another embodiment, the additive is DMSO. In another embodiment, the additive is sucrose. In another embodiment, the additive is any other colligative additive or additive with anti-freeze properties that is known in the art.

In another embodiment, the fermentation media utilized for growing a culture of a Listeria strain is LB. In another embodiment, the nutrient media is TB. In another embodiment, the nutrient media is a defined media. In another embodiment, the nutrient media is peptone based. In another embodiment, the nutrient media is dextrose based. In another embodiment, the nutrient media is tryptic soy both (TSB). In another embodiment, the nutrient media is any other type of nutrient media known in the art.

In one embodiment, a nutrient or fermentation media disclosed herein comprises a yeast extract or any other similarly useful extract available in the art.

In one embodiment of the methods and compositions disclosed herein, the step of growing is performed with a shake flask. In another embodiment, the flask is a baffled shake flask. In another embodiment, the growing is performed with a batch fermenter. In another embodiment, the growing is performed with a stirred tank or flask. In another embodiment, the growing is performed with an airlift fermenter. In another embodiment, the growing is performed with a fed batch. In another embodiment, the growing is performed with a continuous cell reactor. In another embodiment, the growing is performed in a cultibag. In another embodiment, the growing is performed in a single use bioreactor (SUB). In another embodiment, the growing is performed with a Bioreactor that uses wave-like motion. In another embodiment, the growing is performed with an immobilized cell reactor. In another embodiment, the growing is performed with any other means of growing bacteria that is known in the art.

It will be appreciated by a skilled artisan that the terms “reactor,” “bioreactor,” “fermenter,” and “fermentation system” are used interchangeably herein. In one embodiment, the fermentation system disclosed herein is a disposable system. In some embodiments, the entire manufacturing system may be disposable and may be fully enclosed such that sterile connections are made between various components. In another embodiment, the fermentation system is any fermentation system known in the art.

In one embodiment, the term “cultibag” “bioreactor,” “fermenter” and “fermenter system” are used interchangeably herein. In one embodiment, the fermenter disclosed herein is aseptically sampled to measure Optical Density, pH and Viable Cell Count off-line following transfer of the WCB into said fermenter. In another embodiment, the fermenter disclosed herein is aseptically sampled to measure Optical Density, pH and Viable Cell Count off-line following any step of the manufacturing process.

In one embodiment, the fermenter is set at a specific rocking rate. In another embodiment, the bioreactor is set to rock 10-30 times per minute. In another embodiment, the bioreactor is set to rock 20-40 times per minute. In another embodiment, the bioreactor is set to rock 50-80 times per minute.

In another embodiment, the fermenter is set at a specific rocking angle. In another embodiment, the fermenter is set to rock at a 2-10° angle. In another embodiment, the fermenter is set to rock at a 11-20° angle. In another embodiment, the fermenter is set to rock at a 21-40° angle. In another embodiment, the fermenter is set to rock at a 41-60° angle. In another embodiment, the fermenter is set to rock at a 61-80° angle. In another embodiment, the fermenter is set to rock at an 80-90° angle.

In one embodiment, the fermentation process is controlled by monitoring the dissolved oxygen (pO₂) levels, the pH and temperature within the fermentation system. In another embodiment, the pO₂ is monitored during the exponential growth.

In one embodiment, the fermentation process is controlled by monitoring the dissolved oxygen (pO₂) levels, the pH and temperature within the fermentation system at intervals of up to 20 minutes. In another embodiment, the pO₂ is monitored during the exponential growth at intervals of up to 20 minutes. In one embodiment, the fermentation process is controlled by monitoring the dissolved oxygen (pO₂) levels, the pH and temperature within the fermentation system at intervals of up to 40 minutes. In another embodiment, the pO₂ is monitored during the exponential growth at intervals of up to 40 minutes. In one embodiment, the fermentation process is controlled by monitoring the dissolved oxygen (pO₂) levels, the pH and temperature within the fermentation system at intervals of up to 60 minutes. In another embodiment, the pO₂ is monitored during the exponential growth at intervals of up to 60 minutes.

In one embodiment, the fermentation process is controlled by monitoring the dissolved oxygen (pO₂) levels, the pH and temperature within the fermentation system at intervals of more than 60 minutes. In another embodiment, the pO₂ is monitored during the exponential growth at intervals of more than 60 minutes.

In one embodiment, the fermentation process is sampled to measure the optical density (OD 600 nm), the pH, and the viable cell count (VCC).

In one embodiment, the fermentation process is sampled to measure the optical density (OD 600 nm), the pH, and the viable cell count (VCC) at intervals of up to 20 minutes. In another embodiment, the fermentation process is sampled to measure the optical density (OD 600 nm), the pH, and the viable cell count (VCC) at intervals of up to 40 minutes. In another embodiment, the fermentation process is sampled to measure the optical density (OD 600 nm), the pH, and the viable cell count (VCC) at intervals of up to 60 minutes. In another embodiment, the fermentation process is sampled to measure the optical density (OD 600 nm), the pH, and the viable cell count (VCC) at intervals of more than 60 minutes.

In another embodiment, the pH of the fermentation process disclosed herein is controlled using an alkylating agent. In another embodiment, the alkylating agent is selected from one or more of the following sodium hydroxide, potassium carbonate, potassium hydroxide, sodium carbonate, sodium metasilicate, trisodium phosphate. In another embodiment, a constant pH is maintained during growth of the culture in a fermenter system. In another embodiment, the pH is maintained at about 7.0. In another embodiment, the pH is about 6. In another embodiment, the pH is about 6.5. In another embodiment, the pH is about 7.5. In another embodiment, the pH is about 8. In another embodiment, the pH is 6.5-7.5. In another embodiment, the pH is 6-8. In another embodiment, the pH is 6-7. In another embodiment, the pH is 7-8.

In another embodiment, a constant temperature is maintained during growth of the culture. In another embodiment, the temperature is maintained at about 37° C. In another embodiment, the temperature is 37° C. In another embodiment, the temperature is 25° C. In another embodiment, the temperature is 27° C. In another embodiment, the temperature is 28° C. In another embodiment, the temperature is 30° C. In another embodiment, the temperature is 32° C. In another embodiment, the temperature is 33° C. In another embodiment, the temperature is 34° C. In another embodiment, the temperature is 35° C. In another embodiment, the temperature is 36° C. In another embodiment, the temperature is 38° C. In another embodiment, the temperature is 39° C. In another embodiment, the temperature is 40° C. In another embodiment, the temperature is 41° C. In another embodiment, the temperature is 42° C. In another embodiment, the temperature is 43° C. In another embodiment, the temperature is 44° C. In another embodiment, the temperature is 45° C. In another embodiment, the temperature is about 30° C.-45° C.

In one embodiment, a (pO₂) level is monitored during the exponential growth phase of the recombinant Listeria culture. In another embodiment, the pO₂ concentration is maintained during growth of the culture. In another embodiment, a constant dissolved oxygen (pO₂) concentration is maintained during growth of the culture. In another embodiment, the dissolved oxygen concentration is maintained at 20% of saturation. In another embodiment, the concentration is 15% of saturation. In another embodiment, the concentration is 16% of saturation. In another embodiment, the concentration is 18% of saturation. In another embodiment, the concentration is 22% of saturation. In another embodiment, the concentration is 25% of saturation. In another embodiment, the concentration is 30% of saturation. In another embodiment, the concentration is 35% of saturation. In another embodiment, the concentration is 40% of saturation. In another embodiment, the concentration is 45% of saturation. In another embodiment, the concentration is 50% of saturation. In another embodiment, the concentration is 55% of saturation. In another embodiment, the concentration is 60% of saturation. In another embodiment, the concentration is 65% of saturation. In another embodiment, the concentration is 70% of saturation. In another embodiment, the concentration is 75% of saturation. In another embodiment, the concentration is 80% of saturation. In another embodiment, the concentration is 85% of saturation. In another embodiment, the concentration is 90% of saturation. In another embodiment, the concentration is 95% of saturation. In another embodiment, the concentration is 100% of saturation. In another embodiment, the concentration is near 100% of saturation. In another embodiment, the concentration is above 100% of saturation. In another embodiment, the concentration is 100-120% of saturation.

In one embodiment, the fermentation process is discontinued once an OD_{600 nm} value of 1-10 has been reached.

In another embodiment of methods and compositions disclosed herein, the culture is grown in fermentation media having a maximum volume of 20 liters (L) per vessel. In another embodiment, the media has a maximum volume of 200 ml per vessel. In another embodiment, the media has a maximum volume of 300 ml per vessel. In another embodiment, the media has a maximum volume of 500 ml per vessel. In another embodiment, the media has a maximum volume of 750 ml per vessel. In another embodiment, the media has a maximum volume of 1-5 L per vessel. In another embodiment, the media has a maximum volume of 5-10 L per vessel. In another embodiment, the media has a maximum volume of 10-15 L per vessel. In another embodiment, the media has a maximum volume of 15-20 L per vessel.

In another embodiment, the media has a minimum volume of 2 L per vessel. In another embodiment, the media has a minimum volume of 500 ml per vessel. In another embodiment, the media has a minimum volume of 750 ml per vessel. In another embodiment, the media has a minimum volume of 1 L per vessel. In another embodiment, the media has a minimum volume of 1.5 L per vessel. In another embodiment, the media has a minimum volume of 2.5 L per vessel. In another embodiment, the media has a minimum volume of 2 L per vessel. In another embodiment, the media has a minimum volume of 3 L per vessel. In another embodiment, the media has a minimum volume of 4 L per vessel. In another embodiment, the media has a minimum volume of 5 L per vessel. In another embodiment, the media has a minimum volume of 6 L per vessel. In another embodiment, the media has a minimum volume of 8 L per vessel. In another embodiment, the media has a minimum volume of 10 L per vessel.

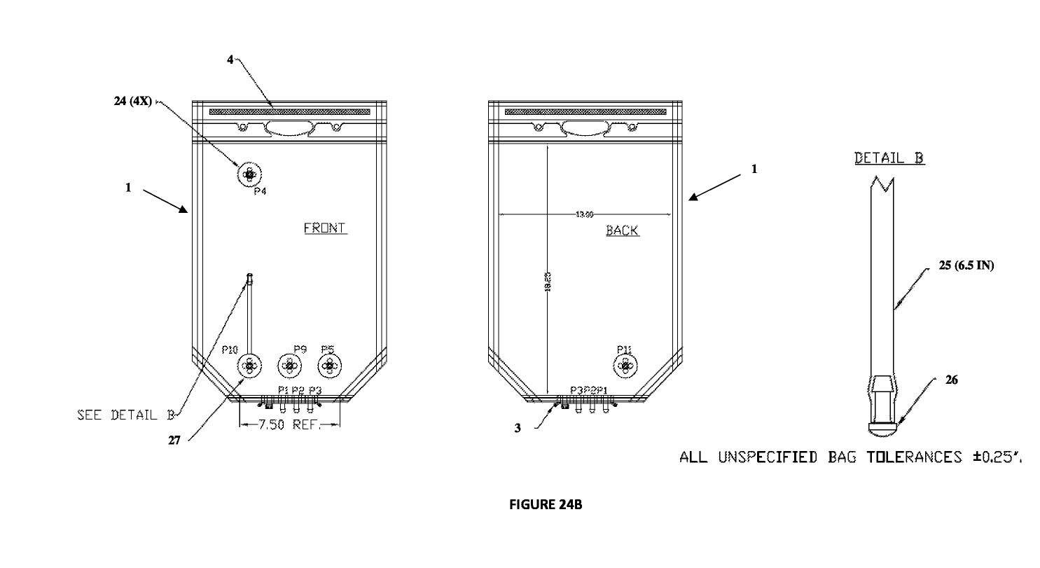

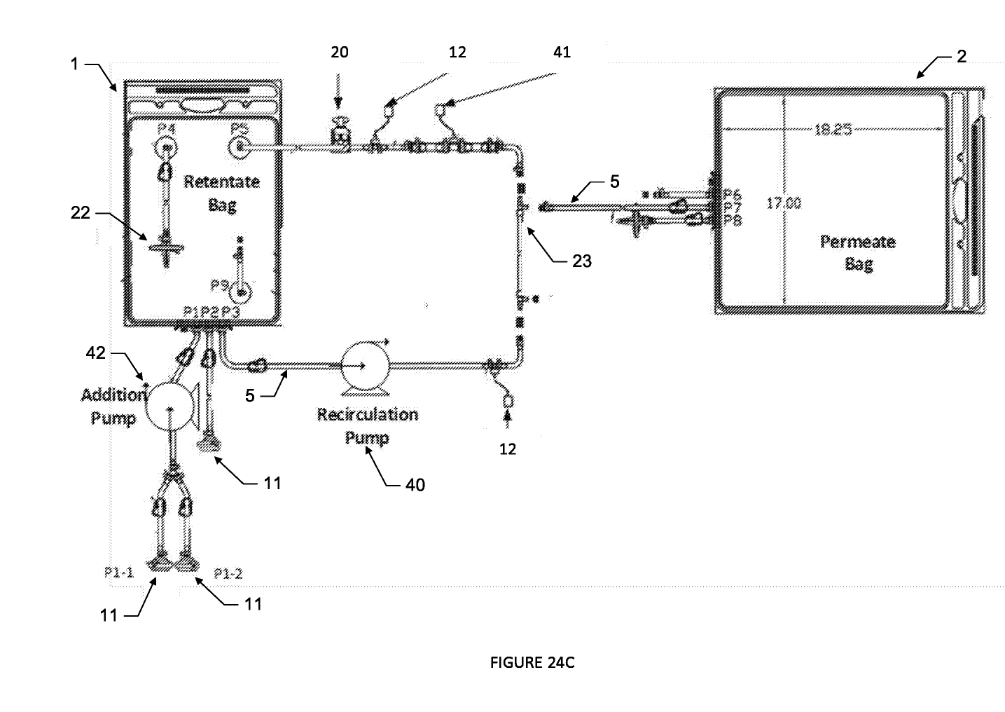

In one embodiment, a recombinant Listeria culture is grown in a fermenter system disclosed herein and is then concentrated using a filtration system. Embodiments of an example filtration system are shown in FIGS. 24A-24C. In another embodiment, a drug substance comprising a recombinant Listeria culture disclosed herein is concentrated using a filtration system, after the Listeria is grown in a fermenter system. In another embodiment, the filtration system is a cross flow filtration (CFF) system or Tangential Flow Filtration (TFF) system. As used herein, the terms cross flow filtration (CFF) system or Tangential Flow Filtration (TFF) system may be used interchangeably. In another embodiment, the fermenter system is aseptically connected to the inlet of the CFF system. In another embodiment, the fermenter system is aseptically connected to the inlet of the TFF system. In another embodiment, the CFF or TFF system is disposable. Each section or component of the manufacturing system disclosed herein may be operably connected with each other section to create a single, fully-enclosed liquid flow path from inoculation, to fermentation, concentration, diafiltration, and to final product dispensation.

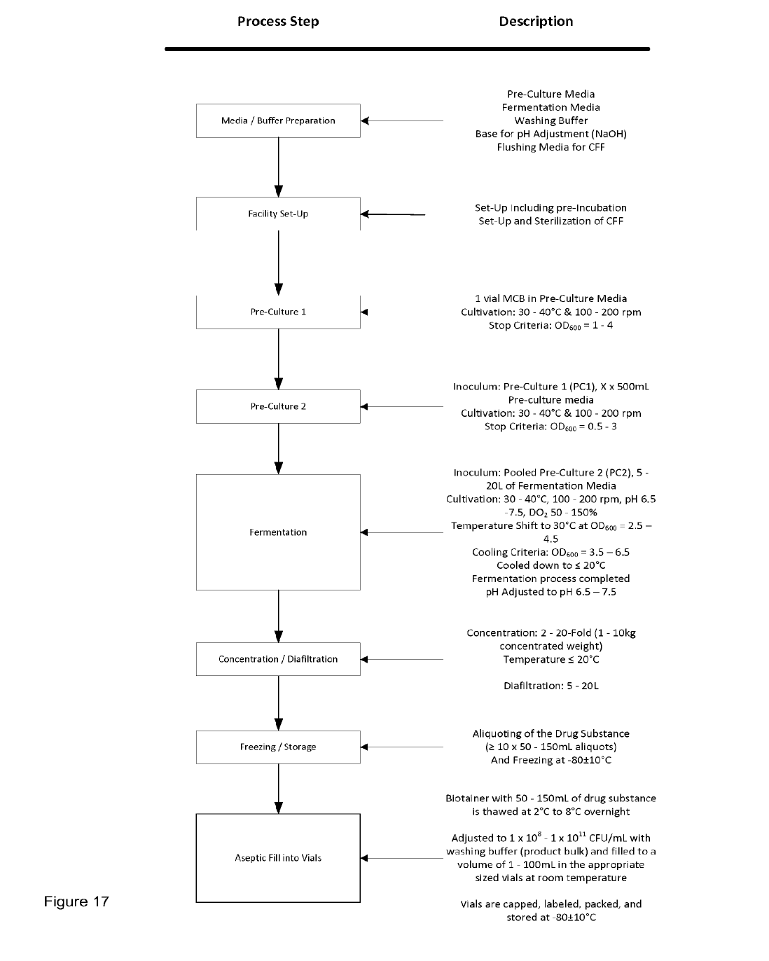

In one embodiment, the manufacturing process is carried out as demonstrated in FIG. 17. In one embodiment, in the beginning stages of the manufacturing process the media/buffer is prepared and a colony containing a Listeria construct is picked from a plate to inoculate a pre-determined volume of fermentation media (in a container suitable for incubation using any of the embodiments discussed herein) and form a first Pre-Culture (PC1). Following incubation of PC1, the culture is up-scaled by obtaining a target volume of PC1 and inoculating into a larger pre-determined volume of fermentation media (in a container suitable for incubation) to form a second Pre-Culture (PC2). In another embodiment, the pre-determined volumes can range from, for example, 10 ml to 300 ml. In another embodiment, a pre-determined volume for PC1 is 10 ml. In another embodiment, a pre-determined volume of PC2 is 500 ml. In another embodiment, the cultures (PC1, PC2) are incubated overnight or at conditions known in the art suitable for growing/incubating bacteria, specifically, Listeria spp.

In another embodiment, following incubation of PC2, a pre-determined volume of PC2 is filled into one or more inoculum bags. In another embodiment, following incubation of PC2, a pre-determined volume of PC2 is filled into inoculum bags (e.g., 4 inoculum bags). In another embodiment, each inoculum bag can hold up to 250 ml. In another embodiment, each inoculum bag can hold up to 500 ml. In another embodiment, each inoculum bag can hold up to 1 L. In another embodiment, each inoculum bag can hold up to 5 L. In another embodiment, each inoculum bag is filled with 25 ml of PC2 and filled up to 100 ml with fermentation media. In another embodiment, each inoculum bag is filled with 1-10 ml of PC2 and filled up to 50-250 ml with fermentation media. In another embodiment, each inoculum bag is filled with 1-20 ml of PC2 and filled up to 50-250 ml with fermentation media. In another embodiment, each inoculum bag is filled with 1-40 ml of PC2 and filled up to 100-500 ml with fermentation media. In another embodiment, each inoculum bag is filled with 1-50 ml of PC2 and filled up to 100-500 ml with fermentation media. In another embodiment, each inoculum bag is filled with 1-100 ml of PC2 and filled up to 150-500 ml with fermentation media. In another embodiment, each inoculum bag is filled with desired volume of PC2 suitable for expanding or upscaling in a larger volume container such as an inoculum bag. In another embodiment, each inoculum bag is filled with desired volume of PC2 suitable for expanding or upscaling in a larger volume container having a predetermined larger volume of fermentation media.

In one embodiment, an inoculum bag containing the expanded Listeria clones, which in one embodiment are referred to herein as the “drug product” or “product,” can be frozen at −70 to −80° C. for later usage.

In another embodiment, following incubation of PC2, a pre-determined volume of PC2 is filled into cell bag bioreactor for initiation of the fermentation process (FIG. 17). In another embodiment, the fermentation process is carried out in the fermentation section of the manufacturing system according to any of the fermentation methods or processes discussed herein. In another embodiment, the fermentation section comprises a cell bag bioreactor.

In one embodiment, a concentration step disclosed herein is carried out at a low 5-10 fold following a fermentation process. In another embodiment, a fermentation media comprising a drug substance disclosed herein is concentrated 2-10 fold, following a fermentation process. In another embodiment, a drug substance is concentrated 2-15 fold. In another embodiment, the drug substance is concentrated 2, 3, 4, 5, 6, 7, 8, 9, 10, 11, 12, 13, 14 or 15 fold.

In one embodiment, following a concentration step disclosed herein a fermentation spent media comprising a drug substance disclosed herein is exchanged with washing buffer. In another embodiment, following concentration step, the retentate comprising the drug substance is diafiltered using washing buffer. In another embodiment, said diafiltering step is followed by sampling and measuring the OD, pH, VCC, and weight of a harvest comprising said drug substance. In another embodiment, said drug substance is diafiltered about 1-10 times (against 1-10 volumes of washing/diafiltration buffer) and then transferred to a biotainer prior to obtaining a sample and diluting the same in order to measure the OD, pH, VCC, and weight of the drug substance. In another embodiment, said drug substance is diafiltered about 1-10 times (against 1-10 volumes of washing buffer) and then transferred to a biotainer prior to aliquoting the drug substance.

In one embodiment, when the culture of recombinant attenuated Listeria has reached a predetermined OD600 or biomass during fermentation, the culture is then transferred to the concentration and diafiltration segment of the fully enclosed cell growth system for carrying out concentration and diafiltration as discussed in any of the embodiments herein.

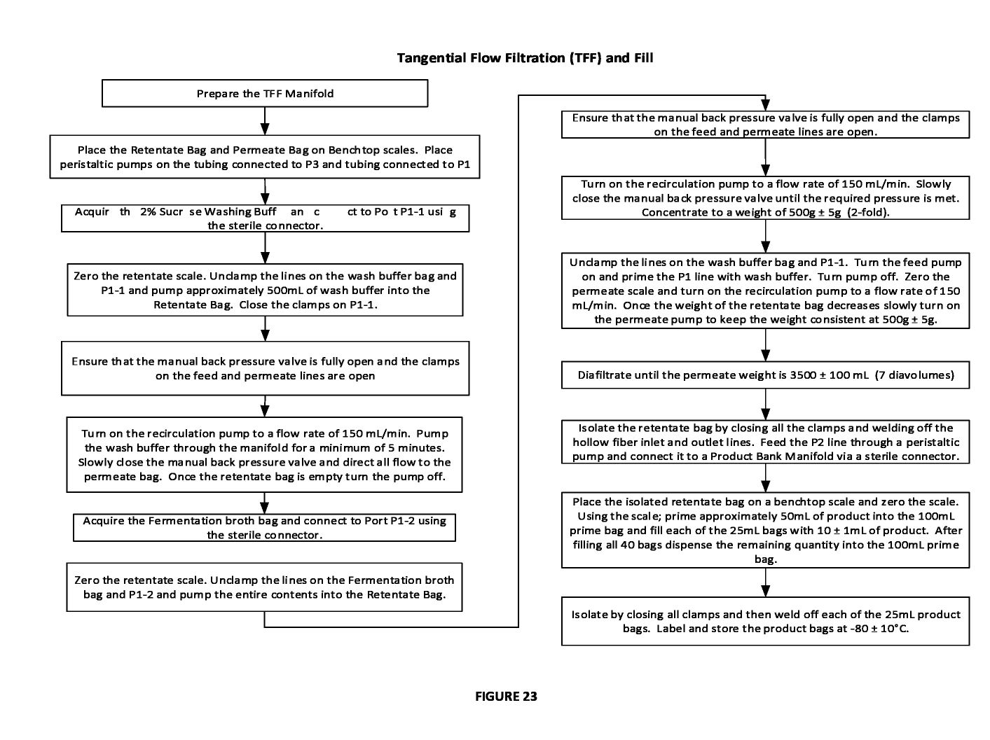

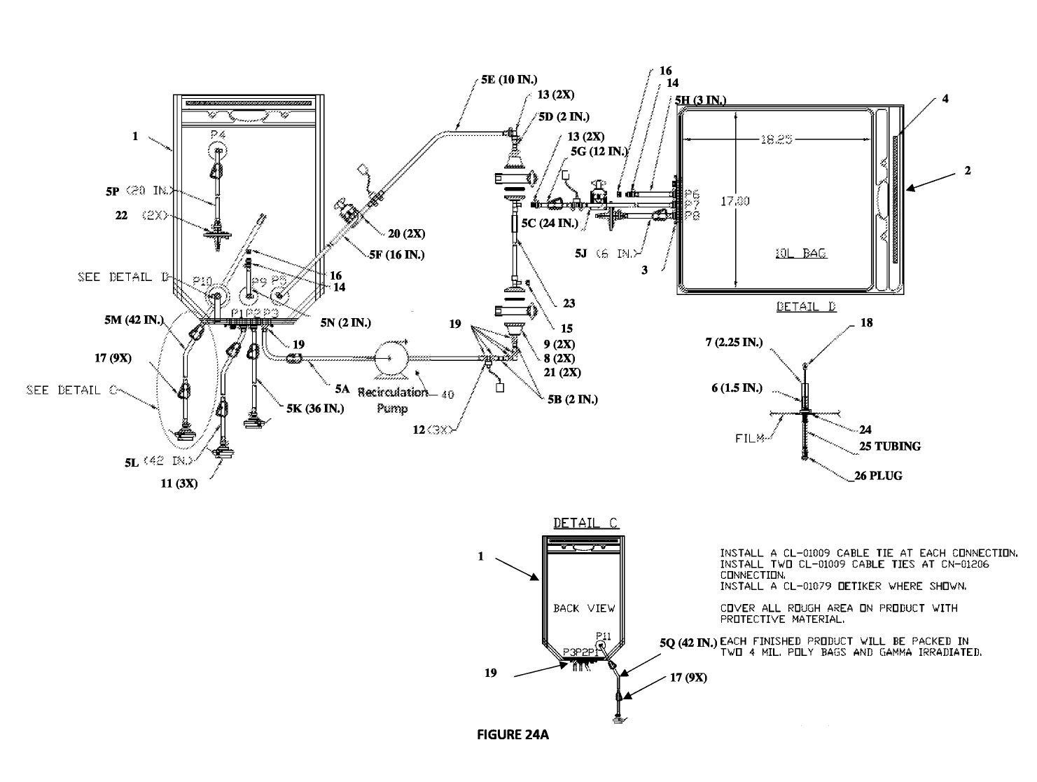

With reference to FIGS. 24A-C, in some embodiments, the concentration and diafiltration section of the disclosed manufacturing system is also referred to as “tangential flow filtration manifold” (TFF) or cross-flow filtration system or manifold (CFF). In one embodiment, the concentration and diafiltration section comprises a concentrated culture container, also called a retentate container 1, one or more filters 23 and a permeate container 2. In another embodiment, said concentration and diafiltration section further comprises one or more fluid conduits 5 (e.g., 5A-5Q, generically referenced as “5”) connecting said concentrated culture container 1 to one or more fermentation containers of the fermentation section (see FIGS. 23, 24A, and 24C). In another embodiment, each fluid of the conduits 5 between the retentate 1 and a fermentation container further comprise means of permanently interrupting fluid flow, such as a clamp 17 or a pinch valve 20. In yet another embodiment, the concentration section further comprises one or more fluid conduits 5 connecting the retentate container 1 to said one or more filters 23. In a further embodiment, fluid conduits 5 connecting the retentate container 1 and said filter 23 form a loop from the retentae container 1 to the filter 23 (e.g., via conduits 5A and 5B) and back to the retentae container 1 from the filter 23 (e.g., via conduits 5D, 5E, and 5F), thereby forming a recirculating loop between the filter and the retentate container. The fluid conduits 5A, 5B which transport fluid from the retentae bag 1 to the filter 23 (e.g., in a counter-clockwise loop in the embodiment shown in FIG. 24A) may optionally comprise a flow actuator, such as a peristaltic pump 40. In yet further embodiment, the fluid conduits 5C, 5D, 5E which transport fluid from the filter 23 back to the retentae bag 1 may further comprise a means of interrupting fluid flow, such as a valve 20 or a clamp 17. In another embodiment, said one or more filters 23 are arranged in a filter array, wherein, in one embodiment, the filters are arranged in series, or, in another embodiment, the filters are arranged in parallel.

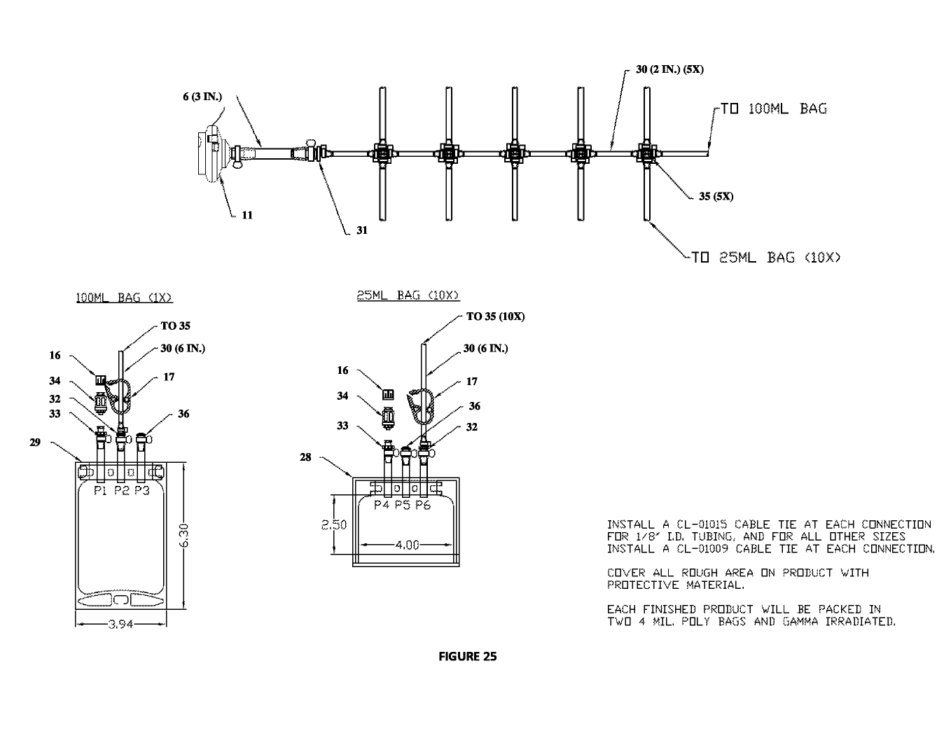

With continued reference to FIGS. 24A-24C, the retentae bag 1 may include a plurality of sterile openings to allow engagement with one or more conduits 5, circulation of the mixtures, and introduction of the diafiltration buffer discussed below. The retentae bag 1 may include a recirculation outlet P3 through which the mixture is drawn from the retentae bag, a recirculation inlet P5 through which the remaining mixture is reintroduced to the retentae bag after passing the filter 23, a diafiltration inlet P11 (shown in Detail C of FIG. 24A) through which the buffer may be introduced. The retentae bag 1 and/or the permeate bag 2 may further include an air exchange device 22 for equalizing the pressure in the respective bags. The air exchange device 22 may include one or more valves and filters for cleaning incoming air and preventing spillage. The retentae bag 1 may further include a thermometer port P10 for receiving a thermometer during operation. With reference to FIG. 25C, in some embodiments a thermometer 41 may be positioned on a conduit 4 of the fluid circulation loop. As detailed herein, the retentae bag 1 may include one or more additional ports P1, P2, P9 for the fermenter or additional features, manifolds, or sampling devices, and similarly, the permeate bag 2 may include one or more ports P6, P7, P8 to which similar air exchange devices, sampling ports, and the filter 23 may be connected. In some embodiments, one or more clamps 8, 9, 17 may be positioned on one or more conduits 5 of the concentration and diafiltration system for controlling the flow therethrough.

As discussed herein, the concentration and diafiltration section shown in FIGS. 24A-24C may, in a concentration step, remove media from the fluid mixture of the construct to concentrate the construct. In the embodiments depicted in FIGS. 24A and 24C, the media passes through the membrane of the filter 23 (e.g., a hollow fiber filter) into the permeate bag 2 as the mixture is pumped from the retentae container 1, through the conduits 5, past the filter 23, and back into the retentae bag 1 by pump 40. By separating the old media, while retaining the construct in the retentae bag 1 and conduits 5, the concentration and diafiltration section may concentrate the construct. For example, the concentration and diafiltration section may perform a 2-fold concentration of the construct. The filter may include at least one filter surface oriented substantially perpendicular to the flow direction in the conduits 5, such that the mixture engages the filter substantially tangentially.

The concentration and diafiltration section may further include a scale (not shown) on which the retentae bag 1 may be positioned. Based on an initial weight of the retentae bag 1 and monitoring of the weight during the concentration process, the change in concentration may be indirectly calculated based on the weight of media removed. In some embodiments, a valve 20 (e.g., a screw valve or pinch valve) may be adjusted either by computer-operated actuators or manually to restrict flow in the conduits 5 and maintain the pressure in the conduits 5 at the filter 23. The mixture in the circulation system may be kept at a predetermined pressure (e.g., 3 psi) to facilitate passage of the medium through the membrane of the filter. In the embodiment shown in FIGS. 24A and 24C, a pressure sensor (e.g., pressure sensor 12 shown in FIG. 24C) is positioned upstream of the pinch valve 20 to effectively measure the pressure in the system between the pump 40 and the valve 20, including the pressure at the filter 23. In one embodiment, the filter array comprises one filter 23. In another embodiment, the filter array comprises more than one filter unit. In yet another embodiment, the filter array comprises two filter units. In yet another embodiment, the filter array comprises three filter units. In yet another embodiment, the filter array comprises four filter units. In yet another embodiment, the filter array comprises five filter units. In yet another embodiment, the filter array comprises more than five filter units.

In one embodiment, the filters 23 are capable of retaining bacteria in the recirculation loop with the retentae bag 1 while allowing fluids, such as the medium to pass through a membrane to the permeate bag 2. In another embodiment, the filters additionally allow macroparticles, such as viral particles and macromolecules to pass through.

In one embodiment, the filters have membrane pore size at least about 0.01-100 μm2. In another embodiment, the filters operate through diafiltration.

The concentration section may further comprise a fluid conduit 5C, 5G connecting the filter 23 to a permeate container 2 (e.g., bag), said fluid conduit further comprising a valve or clamp allowing for unidirectional flow toward the permeate container, and, optionally, further comprising a flow actuator, such as a pump.

In another embodiment, the concentrated culture container 1 and the permeate container 2 are plastic containers. In another embodiment, the concentrated culture container 1 and the permeate container 2 are tissue culture bags.

In one embodiment, the concentrated culture container 1 has a maximum volume of about 100 ml. In another embodiment, the concentrated culture container 1 has a maximum volume of about 150 ml. In another embodiment, the concentrated culture container 1 has a maximum volume of about 200 ml. In another embodiment, the concentrated culture container 1 has a maximum volume of about 250 ml. In another embodiment, the concentrated culture container 1 has a maximum volume of about 300 ml. In another embodiment, the concentrated culture container 1 has a maximum volume of about 350 ml. In another embodiment, the concentrated culture container 1 has a maximum volume of about 400 ml. In another embodiment, the concentrated culture container 1 has a maximum volume of about 450 ml. In another embodiment, the concentrated culture container 1 has a maximum volume of about 500 ml.

In one embodiment, the permeate container 2 has a maximum volume of about 100 ml. In another embodiment, the permeate container 2 has a maximum volume of about 150 ml. In another embodiment, the permeate container 2 has a maximum volume of about 200 ml. In another embodiment, the permeate container 2 has a maximum volume of about 250 ml. In another embodiment, the permeate container 2 has a maximum volume of about 300 ml. In another embodiment, the permeate container 2 has a maximum volume of about 350 ml. In another embodiment, the permeate container 2 has a maximum volume of about 400 ml. In another embodiment, the permeate container has a maximum volume of about 450 ml. In another embodiment, the permeate container 2 has a maximum volume of about 500 ml. In another embodiment, the permeate container 2 has a maximum volume of about 600 ml. In another embodiment, the permeate container 2 has a maximum volume of about 700 ml. In another embodiment, the permeate container 2 has a maximum volume of about 800 ml. In another embodiment, the permeate container 2 has a maximum volume of about 900 ml. In another embodiment, the permeate container 2 has a maximum volume of about 1 L. In another embodiment, the permeate container 2 has a maximum volume of about 1.2 L. In another embodiment, the permeate container 2 has a maximum volume of about 1.4 L. In another embodiment, the permeate container 2 has a maximum volume of about 1.6 L. In another embodiment, the permeate container 2 has a maximum volume of about 1.8 L. In another embodiment, the permeate container 2 has a maximum volume of about 2 L. In another embodiment, the permeate container 2 has a maximum volume of more than 2 L.

In one embodiment, the disclosed culture medium that is transferred from the fermentation section into the retentate container 1 is circulated through a filter array, and the medium that passes through the filters 23 is withdrawn into the permeate container 2, thereby achieving reduced volume of the culture and increasing the concentration of the bacteria in the culture. In another embodiment, the bacteria are concentrated through a single passage over a single use filter array. In some embodiments, the filter 23 includes a hollow fiber filter. In another embodiment, the filtration process uses transmembrane pressure diafiltration to recover cell concentrate. This may differentiate the process disclosed herein from other processes that use transmembrane pressure filtration.

In one embodiment, the final target concentration of bacteria in the culture is about 1-109 bacteria/ml.

In another embodiment, culture of recombinant attenuated Listeria strain is concentrated until the culture's biomass reaches a predetermined value. In one embodiment, the biomass is about 7×109 CFR/ml. In another embodiment, the biomass is about 9×109 CFR/ml. In another embodiment, the biomass is about 10×109 CFR/ml. In another embodiment, the biomass is about 12×109 CFR/ml. In another embodiment, the biomass is about 15×109 CFR/ml. In another embodiment, the biomass is about 20×109 CFR/ml. In another embodiment, the biomass is about 25×109 CFR/ml. In another embodiment, the biomass is about 30×109 CFR/ml. In another embodiment, the biomass is about 33×109 CFR/ml. In another embodiment, the biomass is about 40×109 CFR/ml. In another embodiment, the biomass is about 50×109 CFR/ml. In another embodiment, the biomass is more than 50×109 CFR/ml.

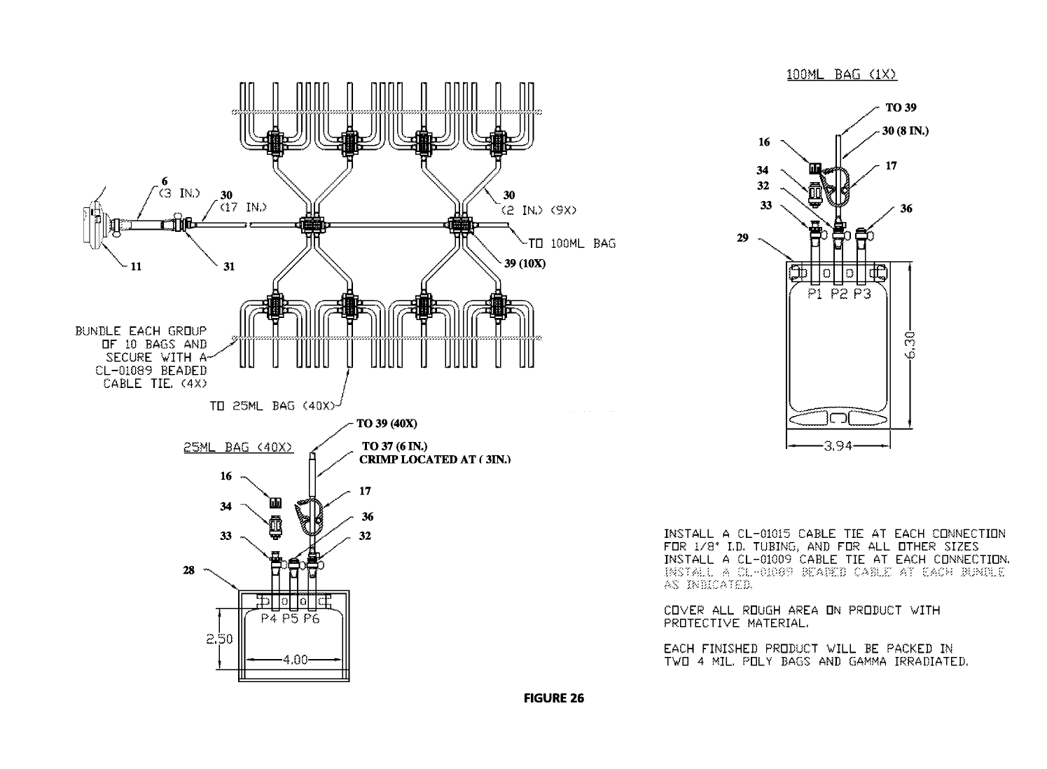

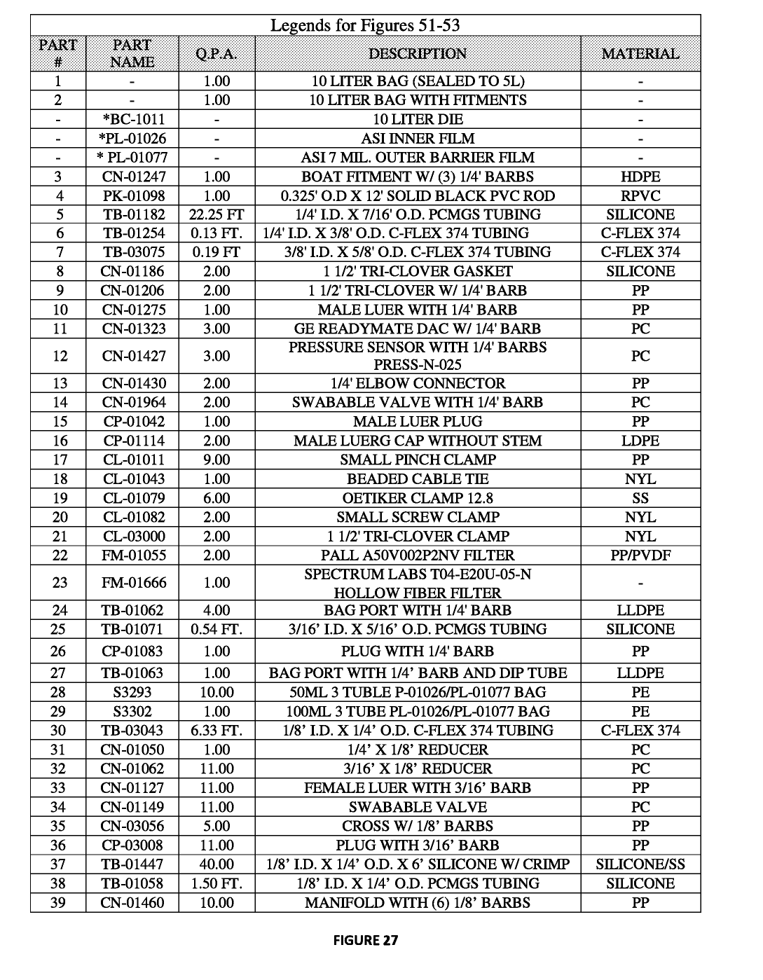

In an additional embodiment, the retentate container further comprises at least one optional port P1, P2 for connecting one or more manifolds (e.g., manifolds 39 shown in FIGS. 25-26) for sampling and/or filling containers of product, similar to sampler ports in the fermentation section and concentration sections.

In one embodiment, the tangential flow filtration manifold comprises a retentate container, a formulation buffer container configured to connect to the retentae container via one or more diafiltration inlets P11; one or more filters 23; and a permeate container 2. In another embodiment, the concentration and diafiltration section further comprises a fluid conduit 5 connecting the permeate container 2 to the retentate container 1 of the concentration and diafiltration section. In yet another embodiment, the concentration and diafiltration section further comprises one or more fluid conduits 5 connecting the retentate container 1 to said one or more filters 23. In a further embodiment, fluid conduits connecting the retentate container 1 and the filters 23 comprise both direct flow conduits 5 configured to carry fluid from the retentae bag 1 to the filter 23 and reverse flow conduits configured to carry fluid from the filter back to the retentae bag, thereby forming a recirculating loop between the filters and the retentate container. In a further embodiment, said direct flow fluid conduits optionally comprise a flow actuator 40, such as a peristaltic pump. In yet further embodiment, said reverse flow fluid conduits further comprise means of slowing or interrupting fluid flow, such as a valve 20 or a clamp 17. In another embodiment, said one or more filters are arranged in a filter array, wherein, in one embodiment, the filters are arranged in series, or, in another embodiment, the filters are arranged in parallel.

After concentrating the construct product during the concentration process, diafiltration may be carried out to clean the product and replace the old media with buffer solution. During diafiltration, a formation buffer container is connected to the retentae bag 1 via the one or more diafiltration inlets P11. The formation buffer container (e.g., a container similar to bags 28, 29) may connect to an aseptic coupling 11 connected via a conduit 5M to the diafiltration inlet P11. Once connected, the formation buffer container may introduce buffer (e.g., Phosphate-Buffered Saline (PBS) buffer) at a controlled rate into the retentae bag 1. The concentration and diafiltration section may continue to circulate the mixture past the filter 23 to remove fluids, including old media, from the mixture. As buffer is introduced, the old media may be diluted while maintaining the overall concentration of construct. In some embodiments, the diafiltration may be manually controlled by squeezing or pumping the buffer into the retentae bag 1. In some embodiments, a computer system (e.g., a controller, microprocessor, or the like, coupled with a non-transitory memory) may control the inlet of buffer. For example, in some embodiments the manual or computerized operator may monitor the scale to maintain a steady weight of the retentae bag 1. With reference to FIG. 24C, an additional pump 42 connected to the conduit 5M may be used to supply the buffer. In some embodiments, the diafiltration may alternately overlap the concentration process, such that at least a portion of the construct is concentrated while new buffer is added.

In some embodiments, the buffer may include a cryoprotectant to protect the construct from freezing damage during later freezing processes. For example, the buffer may include 2% Sucrose. In some alternate embodiments, any solution may be used to achieve the cryoprotectant effect, such as glycerol, glycol compounds, and other cryoprotectants as would be appreciated by one of ordinary skill in the art in light of this disclosure.

In some embodiments, the recirculation outlet P3, the recirculation inlet P5, and/or the diafiltration inlet P11 may be positioned to prevent settling of the construct in the retentae bag. For example, in the depicted embodiment, the recirculation outlet P3 and the diafiltration inlet P11 are positioned proximate the bottom of the retentae bag 1 in its operational position. The recirculation outlet P3 and the diafiltration inlet P11 may be positioned at the bottom of the retentae bag 1. In some embodiments, the recirculation outlet P3 and the diafiltration inlet P11 may be positioned proximate each other to create vortices in the retentae bag 1 and prevent settling. In some embodiments, the recirculation outlet P3 and the diafiltration inlet P11 may be positioned less than one inch from each other. In some embodiments, the recirculation outlet P3 and the diafiltration inlet P11 may be positioned less than two inches from each other. In some embodiments, the recirculation outlet P3 and the diafiltration inlet P11 may be positioned less than three inches from each other. In some embodiments, the recirculation outlet P3 and the diafiltration inlet P11 may be positioned less than four inches from each other. In some alternate embodiments, the recirculation inlet P5 may be positioned proximate at least one of the recirculation outlet P3 and the diafiltration inlet P11 to create vortices.

In some embodiments, the flow rate through the recirculation loop may be maintained at a determined flow rate. The flow rate may be sufficiently high to prevent the formation of biofilms and clogging, and the flow rate may be sufficiently low to prevent shearing and killing the construct. The flow rate may be experimentally established based upon the viscosity of the mixture and filter size/flow rate (e.g., the number of fibers in a hollow fiber filter) and is dependent upon the Reynolds number. In some embodiments, the flow rate may be sufficiently high to cause turbulent flow in the circulation loop, where the turbulent flow helps to prevent biofilm formation. The pump 40 may be controlled manually, preset to a predetermined flow rate, or automatically controlled by a computer system to maintain the flow rate.

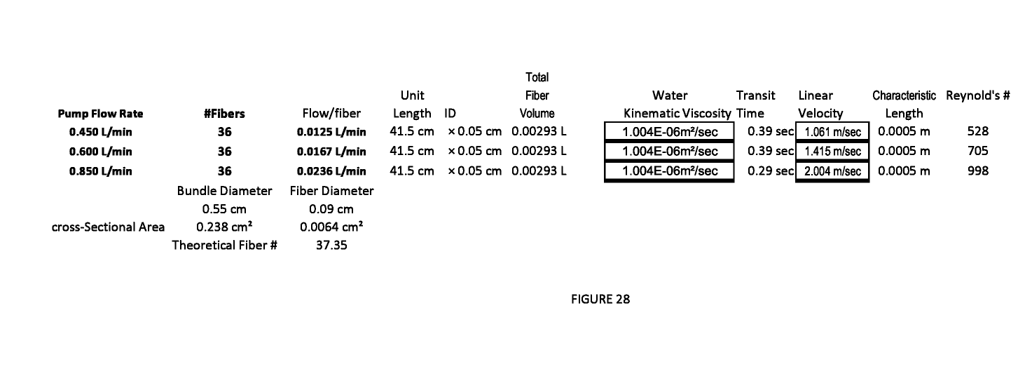

In some embodiments, the flow rate may be from 0.450 L/min to 0.850 L/min. In some embodiments, the flow rate may be from 0.250 L/min to 1 L/min, or any individual sub-increment thereof. In some embodiments, the flow rate may be 0.600 L/min. In some embodiments, the flow rate may be 0.650 L/min. In some embodiments, the flow rate may be from 0.650 L/min to 0.850 L/min. In some embodiments, the flow rate may be from 0.600 L/min to 0.850 L/min. In some embodiments, the flow rate may be from 0.450 L/min to 0.650 L/min. In some embodiments, the flow rate may be from 0.450 L/min to 0.600 L/min. In some embodiments, the flow rate may be from 0.600 L/min to 0.650 L/min. With reference to FIG. 28, a table is shown comparing Reynolds number, pump flow rate, fiber count, velocity, kinematic viscosity, flow/fiber, unit length, internal diameter, fiber volume, and transit time, characteristic length for several example embodiments. In some embodiments, a Reynolds number of approximately 700 is preferred. In some embodiments, the pump speed may remain constant during concentration and diafiltration. In some other embodiments, the pump speed may increase or decrease as the Reynolds number changes. In some embodiments, the pump speed may increase during concentration and/or diafiltration.

As detailed herein, the concentration and diafiltration may be controlled by one or more computer systems including processors, memory, one or more sensors, one or more actuators and associated analysis and control software and hardware as would be understood by one of ordinary skill in the art in light of this disclosure. One or more sensors may be disposed in the concentration and diafiltration section to provide operational data to a user or computer. In some embodiments, the accumulation of biofilm may be detected by one or more pressure sensors (e.g., pressure sensors 12 shown in FIG. 24C) positioned in the conduits 5. A pressure reading may be taken in two or more locations to detect a decrease in pressure in the loop. Detection of a change from a baseline pressure differential may indicate the formation of a biofilm and thus, that the flow rate through the loop is too low. In response to a change in the pressure differential between the two or more pressure sensors, the section may increase the pump speed, or signal an error if the biofilm is not removed. In some embodiments, the two of the pressure sensors may be positioned on either side of the filter 23.

In some embodiments, shearing of the construct may be detected by one or more optical density sensors. In some embodiments, a change in optical density of the mixture from a baseline optical density may indicate shear. The baseline may be taken at the beginning of a concentration or diafiltration step. In some embodiments, a live/dead count may be taken to determine the maximum flow rate.

The optical density sensor may be positioned in the retentae bag 1 or in the conduits 5 to detect the optical density of the circulating mixture. In some embodiments, two or more optical density sensors may be positioned at different locations in the recirculation loop to detect changes in optical density. In some other embodiments, an optical density sensor may be positioned in the permeate bag 2 to detect changes in optical density as part of any of the OD measurements needed or described herein. Typically, the permeate bag 2 will contain little to no construct and will thus have low to no opacity. Sheared construct may pass through the filter 23 rather than recirculating in the concentration loop, and as such, a change (e.g., increase) in optical density of the permeate bag 2 may indicate that shearing is occurring. In response to a change in optical density, the pump 40 speed may be increased by the computer system or user.

In one embodiment, the filter array comprises one filter unit. In another embodiment, the filter array comprises more than one filter unit. In yet another embodiment, the filter array comprises two filter units. In yet another embodiment, the filter array comprises three filter units. In yet another embodiment, the filter array comprises four filter units. In yet another embodiment, the filter array comprises five filter units. In yet another embodiment, the filter array comprises more than five filter units.

A filter disclosed herein may be a bag membrane filter, a flat surface membrane filters, a cartridge filters, an adsorbent filter or absorbent filter. In another embodiment, the filters are hollow fiber filters.

In one embodiment, the filters are capable of retaining bacteria while allowing medium to pass through. In another embodiment, the filters additionally allow macroparticles, such as viral particles and macromolecules to pass through.

In one embodiment, the filters have membrane pore size at least about 0.01-100 μm2. In another embodiment, the filters operate through tangential flow filtration.

In another embodiment, the concentration and diafiltration section further comprises a fluid conduit connecting the filter array to a permeate bag, said fluid conduit further comprising a valve allowing for unidirectional flow toward the permeate container, and, optionally, further comprises a flow actuator, such as a pump. In another embodiment, the concentration and diafiltration section further comprises a fluid conduit connecting the formulation buffer container to a retentate container, said fluid conduit further comprising a valve allowing for unidirectional flow toward the retentate container, and, optionally, further comprising a flow actuator, such as a pump.

In another embodiment, the retentate, formulation buffer, and permeate container are plastic containers. In another embodiment, the retentate, formulation buffer, and permeate container are tissue culture bags.

In one embodiment, the retentate container has a maximum volume of about 100 ml. In another embodiment, the retentate container has a maximum volume of about 150 ml. In another embodiment, the retentate container has a maximum volume of about 200 ml. In another embodiment, the retentate container has a maximum volume of about 250 ml. In another embodiment, the retentate container has a maximum volume of about 300 ml. In another embodiment, the retentate container has a maximum volume of about 350 ml. In another embodiment, the retentate container has a maximum volume of about 400 ml. In another embodiment, the retentate container has a maximum volume of about 450 ml. In another embodiment, the retentate container has a maximum volume of about 500 ml.

In one embodiment, the formulation buffer container has a maximum volume of about 100 ml. In another embodiment, the formulation buffer container has a maximum volume of about 150 ml. In another embodiment, the formulation buffer container has a maximum volume of about 200 ml. In another embodiment, the formulation buffer container has a maximum volume of about 250 ml. In another embodiment, the formulation buffer container has a maximum volume of about 300 ml. In another embodiment, the formulation buffer container has a maximum volume of about 350 ml. In another embodiment, the formulation buffer container has a maximum volume of about 400 ml. In another embodiment, the formulation buffer container has a maximum volume of about 450 ml. In another embodiment, the formulation buffer container has a maximum volume of about 500 ml.

In one embodiment, the formulation buffer container is filled with formulation buffer and integrated into fully enclosed cell growth system prior to the start of the manufacturing process. In another embodiment, the formulation buffer container is filled with formulation buffer and integrated into fully enclosed cell growth system via, for example, a disposable aseptic connector while the manufacturing process is underway.

In another embodiment, the formulation buffer is equated to predetermined temperature prior to use. In another embodiment, both retentate container and formulation buffer container are equated to predetermined temperature prior to diafiltration process. In one embodiment, the temperature is maintained at about 37° C. In another embodiment, the temperature is about 37° C. In another embodiment, the temperature is about 4° C. In another embodiment, the temperature is about 8° C. In another embodiment, the temperature is about 12° C. In another embodiment, the temperature is about 16° C. In another embodiment, the temperature is about 12° C. In another embodiment, the temperature is about 20° C. In another embodiment, the temperature is about 25° C. In another embodiment, the temperature is about 27° C. In another embodiment, the temperature is about 28° C. In another embodiment, the temperature is about 30° C. In another embodiment, the temperature is about 32° C. In another embodiment, the temperature is about 34° C. In another embodiment, the temperature is about 35° C. In another embodiment, the temperature is about 36° C. In another embodiment, the temperature is about 38° C. In another embodiment, the temperature is about 39° C.

In another embodiment, the culture medium transferred from the concentration section into the retentate container 1 is circulated through said filter array, wherein the medium that passed through the filters 23 is withdrawn into the permeate container 2, while at the same time formulation buffer is added to retentate container 1, thereby achieving replacement of nutrient medium with formulation buffer. In another embodiment, the buffer is replaced through a single passage over a single use filter array. In additional embodiment, the volume of the formulation buffer added to retentate bag 1 is less than the medium volume removed in into the permeate container 2, thereby achieving reduced volume of the culture and thus increases concentration of the bacteria in the immunotherapeutic composition. In yet another embodiment, the volume of the formulation buffer added to retentate bag 1 is greater than the medium volume removed in into the permeate container 2, thereby achieving increased volume of the culture and thus decreased concentration of the bacteria in the immunotherapeutic composition. In another embodiment, the filtration process uses transmembrane pressure diafiltration to recover the immunotherapeutic composition. This differentiates the process of the disclosure from other processes that use transmembrane pressure filtration. In one embodiment, the final target concentration of bacteria in the culture is about 1-109 bacteria/ml.

In one embodiment, a desired weight to which the drug substance is concentrated following the filtration is about 1 kg. In one embodiment, the desired weight to which the drug substance is concentrated following connecting said fermenter system to the filtration system or otherwise transferring the drug substance from the fermenter to the filtration system (e.g., the concentration and diafiltration system detailed above) is about 0.01 kg to 0.1 kg. In one embodiment, the desired weight to which the drug substance is concentrated following connecting said fermenter system to the filtration system is about 0.1 kg to 1 kg.

In one embodiment, the desired weight to which the drug substance is concentrated following connecting said fermenter system to the filtration system is about 1 kg-5 kg. In one embodiment, the desired weight to which the drug substance is concentrated following connecting said fermenter system to the filtration system is about 5 kg-10 kg.

In one embodiment, the target OD following diafiltration is 5-10 units. In another embodiment, the target OD following diafiltration is 10-20 units. In another embodiment, the target OD following diafiltration is 20-30 units. In another embodiment, the target OD following diafiltration is 30-40 units. In another embodiment, the target OD following diafiltration is 40-50 units. In another embodiment, the target OD following diafiltration is 50-60 units. In another embodiment, the target OD following diafiltration is 60-80 units. In another embodiment, the target OD following diafiltration is 80-100 units. In another embodiment, the target OD following diafiltration is ≥30.

In another embodiment, said measuring of OD is carried out following aseptically obtaining a sample from said biotainer.

In one embodiment, following diafiltration of a harvest comprising a drug substance disclosed herein the harvest is aseptically transferred into a 50 ml-150 ml biotainer. In another embodiment, following diafiltration of a harvest comprising a drug substance disclosed herein the harvest is aseptically transferred into a 150 ml-250 ml biotainer. In another embodiment, following diafiltration of a harvest comprising a drug substance disclosed herein the harvest is aseptically transferred into a 250 ml-350 ml biotainer. In another embodiment, following diafiltration of a harvest comprising a drug substance disclosed herein the harvest is aseptically transferred into a 350 ml-500 ml biotainer. In another embodiment, following diafiltration of a harvest comprising a drug substance disclosed herein the harvest is aseptically transferred into a 500 ml-1 L biotainer. In another embodiment, following diafiltration of a harvest comprising a drug substance disclosed herein the harvest is aseptically transferred into a 1 L-5 L biotainer. In another embodiment, following diafiltration of a harvest comprising a drug substance disclosed herein the harvest is aseptically transferred into a 5-10 L biotainer. In one embodiment, a biotainer comprising a drug substance disclosed herein is stored at −80° C.±10° C. until they are aseptically filled into vials for clinical use.

In one embodiment, a biotainer with a drug substance is closed completely and transferred for sampling and aliquotation in a Grade A/B cleanroom. In another embodiment, prior to the filling process (in a vial), a viable cell count of a drug substance aliquot is determined for the calculation of the dilution factor and required amount for formulation of the drug substance with the same buffer used for the diafiltration step disclosed herein. In another embodiment, 2-7 days prior to the filling process (in a vial), the viable cell count of one drug substance aliquot is determined for the calculation of the dilution factor and required amount for formulation of the drug substance with the same buffer used for the diafiltration step disclosed herein. In another embodiment, a biotainer with the harvest is weighed before aliquotation and a sample of 5±1 mL is taken to analyze OD₆₀₀, pH, and VCC. In another embodiment, a drug substance is diluted to a target OD prior to aliquoting/filling.

In one embodiment, a required number of drug substance biotainers are thawed at about 5° C.±3° C. for about ≤16 hours prior to aliquoting/filling.

In one embodiment, a drug substance disclosed herein is formulated under aseptic conditions and aseptically filled into vials, for example, by the fully-enclosed manufacturing system described herein and shown in FIGS. 24A-26. In another embodiment, a drug substance disclosed herein is formulated under aseptic conditions and aseptically filled into vials to a desired concentration. In another embodiment, the drug substance is aseptically aliquoted into 1-10 mL vials. In another embodiment, the filling process is carried out at room temperature. In another embodiment, the filling process is carried out at 0-20° C. In another embodiment, the bulk drug substance is aseptically aliquoted into about 10-500, 501-1,000, 1,001-10,000, 10,001-20,000, 20,001-30,000, 30,001-40,000, or 40,001-50,000 1-10 mL vials from a biotainer disclosed herein to make a drug product. In one embodiment, an aliquot is obtained from a drug substance manufactured by a process disclosed herein for storing. In another embodiment, an aliquot is obtained from said drug substance and is stored frozen at ≤−70° C. to −80° C. In another embodiment, an aliquot is obtained from said drug substance for quality control testing. In another embodiment, an aliquot is obtained from said drug substance for testing the stability of said drug substance.

In one embodiment, product containers (e.g., vial(s)) disclosed herein is/are disinfected, inspected, labeled, packaged and distributed to clinical sites. In another embodiment, the vials are stored at ≤−90° C. and thawed at room temperature prior to human use.

In one embodiment of methods and compositions of disclosed herein, the immunotherapeutic composition comprising a recombinant attenuated Listeria in formulation buffer is subsequently transferred from the retentate container 1 to the product dispensation section of the fully enclosed cell growth system through aforementioned fluid conduit, said fluid conduit comprising a valve 20 allowing for unidirectional flow toward the product dispensation section (FIG. 26), a means of permanently interrupting the fluid flow, such as a valve 20 or a clamp 17 and, optionally, further comprising a flow actuator, such as a pump.

In one embodiment, the product dispensation section 39 of the manufacturing system disclosed herein is also referred to as a “product bank manifold” or “manifold” (see FIGS. 25-26). In one embodiment, the product dispensation section comprises a bulk container (e.g., retentae container 1), a purge container, and one or more product containers in to which the product may be aliquoted. In yet another embodiment, the product dispensation section further comprises one or more fluid conduits 30 connecting in series the bulk container to said purge container (e.g., 100 mL bag 29) and to said one or more product containers (e.g., 25 mL bags 28), wherein the purge container is positioned at the distal terminus of the series of connections, while the product containers have intermediate position in the series of connections. In a further embodiment, the conduit connecting the bulk container, the purge container and the product containers further comprises means of permanently interrupting flow into each product container, such as a valve 20, a clamp 17 or means for permanently sealing off the conduit, and, optionally, comprises a flow actuator, such as a pump, wherein said actuator positioned proximally to the bulk container. The manifold 39 may aseptically attach to the retentae bag (e.g., P1 or P2 of retentae bag 1 shown in FIGS. 24A-24C) with one or more connectors 11.

In one embodiment, the bulk container and purge container are plastic containers. In another embodiment, the bulk container and purge container are tissue culture bags.

In one embodiment, the product containers are plastic containers, plastic ampoules, glass ampoules or single-use syringes. In another embodiment, the product containers are IV bags further comprising IV delivery port. In another embodiment, the product containers are single dose IV bags.

In one embodiment, the product dispensation section, also referred to herein as “product bank manifold” comprises one single dose product container. In another embodiment, the product dispensation section comprises two single dose product containers. In another embodiment, the product dispensation section comprises three single dose product containers. In another embodiment, the product dispensation section comprises four single dose product containers. In another embodiment, the product dispensation section comprises five single dose product containers. In another embodiment, the product dispensation section comprises six single dose product containers. In another embodiment, the product dispensation section comprises seven single dose product containers. In another embodiment, the product dispensation section comprises eight single dose product containers. In another embodiment, the product dispensation section comprises nine single dose product containers. In another embodiment, the product dispensation section comprises ten single dose product containers. In another embodiment, the product dispensation section comprises more than ten single dose product containers.

In one embodiment, each product container has a volume of about 1-500 ml.

In an additional embodiment, the bulk container comprises at least one optional sampler port similar to sampler ports in the fermentation and concentration/diafiltration sections.

In another embodiment, said fully enclosed cell growth system disclosed herein has a centralized architecture, wherein the fermentation container of the fermentation section also functions as a retentate container of concentration section and diafiltration section, and as bulk container of the product dispensation section. In another embodiment, the centralized fully enclosed cell growth system further comprises separate sets of outgoing fluid conduits connecting fermentation/concentrated culture/retentate/bulk container to the respective components of each of inoculation, concentration/diafiltration and product dispensation section, specifically to inoculation container, to one or more filters of the concentration section/diafiltration section, and to the product and purge containers of product dispensation section. In another embodiment, the centralized fully enclosed cell growth system further comprises a set of recirculation conduits connecting one or more filters of concentration/diafiltration section to fermentation/concentrated culture/retentate/bulk container. In another embodiment, the outgoing fluid conduits connecting said fermentation/concentrated culture/retentate/bulk container to other sections of the centralized fully enclosed cell growth system further comprise optional valves allowing for unidirectional flow away from the fermentation/concentrated culture/retentate/bulk container. In another embodiment, one or more of the outgoing fluid conduits optionally comprise fluid flow actuator, such as a pump. In an additional embodiment, the recirculation conduits connecting said one or more filters of concentration section/diafiltration section to the fermentation/concentrated culture/retentate/bulk container further comprise optional valves allowing for unidirectional flow toward from the fermentation/concentrated culture/retentate/bulk container. In another embodiment, every fluid conduit connected to the fermentation/concentrated culture/retentate/bulk container of the centralized fully enclosed cell growth system further comprised means of permanently interrupting the flow of fluid, such as a valve 20 or a clamp 17, or means of permanently sealing of the conduit.

Disclosed herein is a process for scaling up the process of manufacturing personalized immunotherapeutic compositions through the parallel use of several fully enclosed disposable cell growth systems described hereinabove. In one embodiment, a set of the fully enclosed cell growth systems is used to make several different personalized immunotherapeutic compositions for the same patient. In another embodiment, a set of the fully enclosed cell growth systems is used to make several different personalized immunotherapeutic compositions for the different patients. In another embodiment, parallel use of a set of fully enclosed cell growth systems allows for tremendous increase in the output of personalized immunotherapeutic compositions In one embodiment, said set comprises two fully enclosed cell growth systems operating in parallel. In another embodiment, the set comprises three fully enclosed cell growth systems operating in parallel. In another embodiment, the set comprises four fully enclosed cell growth systems operating in parallel. In another embodiment, the set comprises five fully enclosed cell growth systems operating in parallel. In another embodiment, the set comprises six fully enclosed cell growth systems operating in parallel. In another embodiment, the set comprises seven fully enclosed cell growth systems operating in parallel. In another embodiment, the set comprises eight fully enclosed cell growth systems operating in parallel. In another embodiment, the set comprises nine fully enclosed cell growth systems operating in parallel. In another embodiment, the set comprises ten fully enclosed cell growth systems operating in parallel. In another embodiment, the set comprises more than ten fully enclosed cell growth systems operating in parallel.

Disclosed herein is a process for operating the fully enclosed disposable cell growth system or a set of the systems in a closed environmental chamber. In one embodiment, the closed environmental chamber is a clean room. In another embodiment, the closed environmental chamber is a bio-hood.

In one embodiment, the term “closed environmental chamber” refers to an enclosure of any size that is fully or partially sealed or isolated from the outside environment and wherein one or more environmental parameters such as temperature, pressure, atmosphere, and levels of particulate matter in the air are maintained at particular preset levels.

In another embodiment, the method of manufacturing personalized immunotherapeutic compositions further provides for testing of the compositions being manufactured either concurrently with the manufacturing process, or after the completion of manufacturing process. The concurrent testing can be carried out at any step of manufacturing process and provides significant advantages of continuously monitoring quality of the product throughout the manufacturing process. Concurrent testing further provides an additional advantage of eliminating post-production testing, resulting in significant time savings. In one embodiment, said testing includes, but not limited to purity control, safety control, potency control, identity control and stability control.

In one embodiment, the term “purity control” means testing the personalized immunotherapeutic composition for the presence of process impurities, such as residual media components, product impurities, and contaminating adventurous agents, such as bacteriophages.

In another embodiment, the term “safety control” means testing the personalized immunotherapeutic composition for virulence, specifically, in the case of Listeria, the manufactured composition will be tested for attenuation. In another embodiment, the term “identity control” refers to testing the personalized immunotherapeutic composition for the presence of expected quality attributes, such as antibiotic sensitivity. In another embodiment, the term “potency control” refers to testing the personalized immunotherapeutic composition for therapeutic effectiveness. Therapeutic effectiveness can be tested for example in a model in vitro system.

In another embodiment, the term “stability control” means testing the personalized immunotherapeutic composition for the ability to maintain quality attributes through expected usage.

Disclosed herein is a manufacture-to-order, allowing for delivery of the personalized immunogenic composition to the patient immediately upon completion of manufacturing process. In one embodiment, at least one single dose product container, preferably an IV bag, is detached from single use fully enclosed cell growth system once the product has been delivered to the product container, and the fluid conduit connecting the product container to the cell growth system has been permanently sealed off.

Following the separation the product container is used to directly administer the personalized immunotherapeutic composition to a patient, for example via IV infusion.

Disclosed herein is a system for storing the personalized immunotherapeutic composition for subsequent use or shipment to a patient in a remote location. As contemplated by this disclosure one or more single dose product containers, preferably single use IV bags, are detached from single use fully enclosed cell growth system once the product has been delivered to the product containers, and the fluid conduits connecting the product containers to the cell growth system have been permanently sealed off. Following the separation the product containers are immediately frozen and either stored or shipped. In one embodiment, the personalized immunogenic compositions are frozen, stored and shipped at the temperature below −20 degrees Celsius. In another embodiment, the temperature is about −70 degrees Celsius. In another embodiment, the temperature is about −70-−80 degrees Celsius. In another embodiment, the personalized immunotherapeutic composition is thawed and the bacterial cells are resuspended evenly in the formulation buffer immediately prior to delivery to a patient. In one embodiment, the personalized immunotherapeutic composition is equated to a predetermined temperature immediately prior to delivery to patient. In another embodiment, the temperature is ambient temperature. In another embodiment, the temperature is about 37 degrees Celsius.

In one embodiment, the manufacturing process of disclosed herein eliminates the need to transfer the drug substance to a separate facility for further processing (i.e. filling into vials) thereby reducing the risk of contamination and time. In another embodiment, manufacturing process of disclosed herein allows for manufacture in a Grade D/Class 100,000/ISO 8 or higher environment.

As provided by disclosed herein, the manufacturing step will take up no longer than two weeks. In another embodiment, the manufacturing step will take up about 1-2 weeks. In another embodiment, the manufacturing step will take up about 1 week. In another embodiment, the manufacturing step will take up less than 1 week.

As further provided by disclosed herein, the pre-release testing of immunotherapeutic agent and release step will take up no longer than five weeks. In another embodiment, the pre-release testing of immunotherapeutic agent and release step will take up about 4-5 weeks. In another embodiment, the pre-release testing of immunotherapeutic agent and release step will take up about 4 weeks. In another embodiment, the pre-release testing of immunotherapeutic agent and release step will take up less than 4 weeks.

As additionally provided by disclosed herein, the shipping step will take up no longer than one week. In another embodiment, the shipping step will take up less than 1 week.

In another embodiment of methods and compositions disclosed herein, the step of measuring, sampling, freezing or lyophilizing is performed when the culture has an OD₆₀₀ of 0.1 units. In another embodiment, the culture has an OD₆₀₀ of 0.8 units. In another embodiment, the culture has an OD₆₀₀ of 0.2 units. In another embodiment, the culture has an OD₆₀₀ of 0.3 units. In another embodiment, the culture has an OD₆₀₀ of 0.4 units. In another embodiment, the culture has an OD₆₀₀ of 0.5 units. In another embodiment, the culture has an OD₆₀₀ of 0.6 units. In another embodiment, the OD₆₀₀ is about 0.7 units. In another embodiment, the OD₆₀₀ is about 0.8 units. In another embodiment, the OD₆₀₀ is 0.6 units. In another embodiment, the OD₆₀₀ is 0.65 units. In another embodiment, the OD₆₀₀ is 0.75 units. In another embodiment, the OD₆₀₀ is 0.85 units. In another embodiment, the OD₆₀₀ is 0.9 units. In another embodiment, the OD₆₀₀ is 1 unit. In another embodiment, the OD₆₀₀ is 0.6-0.9 units. In another embodiment, the OD₆₀₀ is 0.65-0.9 units. In another embodiment, the OD₆₀₀ is 0.7-0.9 units. In another embodiment, the OD₆₀₀ is 0.75-0.9 units. In another embodiment, the OD₆₀₀ is 0.8-0.9 units. In another embodiment, the OD₆₀₀ is 0.75-1 units. In another embodiment, the OD₆₀₀ is 0.9-1 units. In another embodiment, the OD₆₀₀ is greater than 1 unit. In another embodiment, the OD₆₀₀ is significantly greater than 1 unit (e.g. when the culture is produced in a batch fermenter). In another embodiment, the OD₆₀₀ is 7.5-8.5 units. In another embodiment, the OD₆₀₀ is 1.2 units. In another embodiment, the OD₆₀₀ is 1.5 units. In another embodiment, the OD₆₀₀ is 2 units. In another embodiment, the OD₆₀₀ is 2.5 units. In another embodiment, the OD₆₀₀ is 3 units. In another embodiment, the OD₆₀₀ is 3.5 units. In another embodiment, the OD₆₀₀ is 4 units. In another embodiment, the OD₆₀₀ is 4.5 units. In another embodiment, the OD₆₀₀ is 5 units. In another embodiment, the OD₆₀₀ is 5.5 units. In another embodiment, the OD₆₀₀ is 6 units. In another embodiment, the OD₆₀₀ is 6.5 units. In another embodiment, the OD₆₀₀ is 7 units. In another embodiment, the OD₆₀₀ is 7.5 units. In another embodiment, the OD₆₀₀ is 8 units. In another embodiment, the OD₆₀₀ is 8.5 units. In another embodiment, the OD₆₀₀ is 9 units. In another embodiment, the OD₆₀₀ is 9.5 units. In another embodiment, the OD₆₀₀ is 10 units. In another embodiment, the OD₆₀₀ is more than 10 units.

In another embodiment, the OD₆₀₀ is 1-2 units. In another embodiment, the OD₆₀₀ is 1.5-2.5 units. In another embodiment, the OD₆₀₀ is 2-3 units. In another embodiment, the OD₆₀₀ is 2.5-3.5 units. In another embodiment, the OD₆₀₀ is 3-4 units. In another embodiment, the OD₆₀₀ is 3.5-4.5 units. In another embodiment, the OD₆₀₀ is 4-5 units. In another embodiment, the OD₆₀₀ is 4.5-5.5 units. In another embodiment, the OD₆₀₀ is 5-6 units. In another embodiment, the OD₆₀₀ is 5.5-6.5 units. In another embodiment, the OD₆₀₀ is 1-3 units. In another embodiment, the OD₆₀₀ is 1.5-3.5 units. In another embodiment, the OD₆₀₀ is 2-4 units. In another embodiment, the OD₆₀₀ is 2.5-4.5 units. In another embodiment, the OD₆₀₀ is 3-5 units. In another embodiment, the OD₆₀₀ is 4-6 units. In another embodiment, the OD₆₀₀ is 5-7 units. In another embodiment, the OD₆₀₀ is 2-5 units. In another embodiment, the OD₆₀₀ is 3-6 units. In another embodiment, the OD₆₀₀ is 4-7 units. In another embodiment, the OD₆₀₀ is 5-8 units. In another embodiment, the OD₆₀₀ is 1.2-7.5 units. In another embodiment, the OD₆₀₀ is 1.5-7.5 units. In another embodiment, the OD₆₀₀ is 2-7.5 units. In another embodiment, the OD₆₀₀ is 2.5-7.5 units. In another embodiment, the OD₆₀₀ is 3-7.5 units. In another embodiment, the OD₆₀₀ is 3.5-7.5 units. In another embodiment, the OD₆₀₀ is 4-7.5 units. In another embodiment, the OD₆₀₀ is 4.5-7.5 units. In another embodiment, the OD₆₀₀ is 5-7.5 units. In another embodiment, the OD₆₀₀ is 5.5-7.5 units. In another embodiment, the OD₆₀₀ is 6-7.5 units. In another embodiment, the OD₆₀₀ is 6.5-7.5 units. In another embodiment, the OD₆₀₀ is 7-7.5 units. In another embodiment, the OD₆₀₀ is more than 10 units. In another embodiment, the OD₆₀₀ is 1.2-8.5 units. In another embodiment, the OD₆₀₀ is 1.5-8.5 units. In another embodiment, the OD₆₀₀ is 2-8.5 units. In another embodiment, the OD₆₀₀ is 2.5-8.5 units. In another embodiment, the OD₆₀₀ is 3-8.5 units. In another embodiment, the OD₆₀₀ is 3.5-8.5 units. In another embodiment, the OD₆₀₀ is 4-8.5 units. In another embodiment, the OD₆₀₀ is 4.5-8.5 units. In another embodiment, the OD₆₀₀ is 5-8.5 units. In another embodiment, the OD₆₀₀ is 5.5-8.5 units. In another embodiment, the OD₆₀₀ is 6-8.5 units. In another embodiment, the OD₆₀₀ is 6.5-8.5 units. In another embodiment, the OD₆₀₀ is 7-8.5 units. In another embodiment, the OD₆₀₀ is 7.5-8.5 units. In another embodiment, the OD₆₀₀ is 8-8.5 units. In another embodiment, the OD₆₀₀ is 9.5-8.5 units. In another embodiment, the OD₆₀₀ is 10 units.

In one embodiment, an OD_{600 nm} analysis is performed to calculate the amount of Formulation Buffer that is needed to achieve a final desired OD of about 5-10 at 600 nm.

In another embodiment, the step of freezing or lyophilization is performed when the culture has a biomass of 1×10⁸-1×10¹¹ colony-forming units (CFU)/ml. In another embodiment, the biomass ranges from 1.0×10⁵ to 1.0×10¹¹ CFU/ml

In another embodiment of methods and compositions disclosed herein, the Listeria culture is flash-frozen in liquid nitrogen, followed by storage at the final freezing temperature. In another embodiment, the culture is frozen in a more gradual manner; e.g. by placing in a vial of the culture in the final storage temperature. In another embodiment, the culture is frozen by any other method known in the art for freezing a bacterial culture.

In another embodiment of methods and compositions disclosed herein, the storage temperature of the culture is between −20 and −90 degrees Celsius (° C.). In another embodiment, the temperature is significantly below −20° C. In another embodiment, the temperature is not warmer than −70° C. In another embodiment, the temperature is −70° C. In another embodiment, the temperature is about −70° C. In another embodiment, the temperature is −20° C. In another embodiment, the temperature is about −20° C. In another embodiment, the temperature is −30° C. In another embodiment, the temperature is −40° C. In another embodiment, the temperature is −50° C. In another embodiment, the temperature is −60° C. In another embodiment, the temperature is −90° C. In another embodiment, the temperature is −30-−70° C. In another embodiment, the temperature is −40-−70° C. In another embodiment, the temperature is −50-−70° C. In another embodiment, the temperature is −60-−70° C. In another embodiment, the temperature is −30-−90° C. In another embodiment, the temperature is −40-−80° C. In another embodiment, the temperature is −50-−90° C. In another embodiment, the temperature is −60-−90° C. In another embodiment, the temperature is −70-−90° C. In another embodiment, the temperature is colder than −70° C. In another embodiment, the temperature is colder than 90° C.

In another embodiment of methods and compositions disclosed herein, the cryopreservation, frozen storage, or lyophilization is for a maximum of 24 hours. In another embodiment, the cryopreservation, frozen storage, or lyophilization is for maximum of 2 days. In another embodiment, the cryopreservation, frozen storage, or lyophilization is for maximum of 3 days. In another embodiment, the cryopreservation, frozen storage, or lyophilization is for maximum of 4 days. In another embodiment, the cryopreservation, frozen storage, or lyophilization is for maximum of 1 week. In another embodiment, the cryopreservation, frozen storage, or lyophilization is for maximum of 2 weeks. In another embodiment, the cryopreservation, frozen storage, or lyophilization is for maximum of 3 weeks. In another embodiment, the cryopreservation, frozen storage, or lyophilization is for maximum of 1 month. In another embodiment, the cryopreservation, frozen storage, or lyophilization is for maximum of 2 months. In another embodiment, the cryopreservation, frozen storage, or lyophilization is for maximum of 3 months. In another embodiment, the cryopreservation, frozen storage, or lyophilization is for maximum of 5 months. In another embodiment, the cryopreservation, frozen storage, or lyophilization is for maximum of 6 months. In another embodiment, the cryopreservation, frozen storage, or lyophilization is for maximum of 9 months. In another embodiment, the cryopreservation, frozen storage, or lyophilization is for maximum of 1 year.

In another embodiment, the cryopreservation, frozen storage, or lyophilization is for a minimum of 1 week. In another embodiment, the cryopreservation, frozen storage, or lyophilization is for minimum of 2 weeks. In another embodiment, the cryopreservation, frozen storage, or lyophilization is for minimum of 3 weeks. In another embodiment, the cryopreservation, frozen storage, or lyophilization is for minimum of 1 month. In another embodiment, the cryopreservation, frozen storage, or lyophilization is for minimum of 2 months. In another embodiment, the cryopreservation, frozen storage, or lyophilization is for minimum of 3 months. In another embodiment, the cryopreservation, frozen storage, or lyophilization is for minimum of 5 months. In another embodiment, the cryopreservation, frozen storage, or lyophilization is for minimum of 6 months. In another embodiment, the cryopreservation, frozen storage, or lyophilization is for minimum of 9 months. In another embodiment, the cryopreservation, frozen storage, or lyophilization is for minimum of 1 year. In another embodiment, the cryopreservation, frozen storage, or lyophilization is for minimum of 1.5 years. In another embodiment, the cryopreservation, frozen storage, or lyophilization is for minimum of 2 years. In another embodiment, the cryopreservation, frozen storage, or lyophilization is for minimum of 3 years. In another embodiment, the cryopreservation, frozen storage, or lyophilization is for minimum of 5 years. In another embodiment, the cryopreservation, frozen storage, or lyophilization is for minimum of 7 years. In another embodiment, the cryopreservation, frozen storage, or lyophilization is for minimum of 10 years. In another embodiment, the cryopreservation, frozen storage, or lyophilization is for longer than 10 years.

In another embodiment of the methods and compositions disclosed herein, the Listeria bacteria exhibit exponential growth essentially immediately after thawing following an extended period of cryopreservation or frozen storage. In another embodiment, the Listeria bacteria exhibit exponential growth essentially immediately after reconstitution following an extended period of lyophilization. In another embodiment, “essentially immediately” refers to within about 1 hour after inoculating fresh media with cells from the cell bank or starter culture. In another embodiment, the bacteria exhibit exponential growth shortly after (e.g. in various embodiments, after 10 minutes (min), 20 min, 30 min, 40 min, 50 min, 1 hour, 75 min, 90 min, 105 min, or 2 hours) thawing following the period of cryopreservation or storage.

The “extended period” of cryopreservation, frozen storage, or lyophilization is, in another embodiment, 1 month. In another embodiment, the period is 2 months. In another embodiment, the period is 3 months. In another embodiment, the period is 5 months. In another embodiment, the period is 6 months. In another embodiment, the period is 9 months. In another embodiment, the period is 1 year. In another embodiment, the period is 1.5 years. In another embodiment, the period is 2 years.

In another embodiment, “exponential growth” refers to a doubling time that is close to the maximum observed for the conditions (e.g. media type, temperature, etc.) in which the culture is growing. In another embodiment, “exponential growth” refers to a doubling time that is reasonable constant several hours (e.g. 1 hour, 1.5 hours, 2 hours, or 2.5 hours) after dilution of the culture; optionally following a brief recovery period.

In another embodiment, a Listeria immunotherapy strain of methods and compositions of the present disclosure retains a viability of over 90% after thawing following 14 days of cryopreservation. In another embodiment, the viability upon thawing is close to 100% following the period of cryopreservation. In another embodiment, the viability upon thawing is about 90%. In another embodiment, the viability upon thawing is close to 90%. In another embodiment, the viability upon thawing is at least 90%. In another embodiment, the viability upon thawing is over 80%.

In another embodiment, a Listeria immunotherapy strain of methods and compositions of the present disclosure retains a viability of over 90% after reconstitution following lyophilization. In another embodiment, the viability upon thawing is close to 100% following the period of lyophilization. In another embodiment, the viability upon thawing is about 90%. In another embodiment, the viability upon thawing is close to 90%.

In another embodiment, the viability upon thawing is at least 90%. In another embodiment, the viability upon thawing is over 80%.

In another embodiment, a cell bank, frozen stock, or batch of immunotherapyimmunotherapy doses of the present disclosure is grown in a defined microbiological media, comprising: (1) between about 0.3 and about 0.6 g/L of methionine; and (2) effective amounts of: (a) cysteine; (b) a pH buffer; (c) a carbohydrate; (d) a divalent cation; (e) ferric or ferrous ions; (f) glutamine or another nitrogen source; (g) riboflavin; (h) thioctic acid (also known as lipoic acid); (i) another or more components selected from leucine, isoleucine, valine, arginine, histidine, tryptophan, and phenylalanine; (j) one or more components selected from adenine, biotin, thiamine, pyridoxal, para-aminobenzoic acid, pantothenate, and nicotinamide; (k) an oxygen source; and (1) one or more components selected from cobalt, copper, boron, manganese, molybdenum, zinc, calcium, and citrate.

In another embodiment, the cell bank, frozen stock, or batch of immunotherapy doses is grown in a defined microbiological media, comprising: (1) between about 0.3 and about 0.6 g/L of cysteine; and (2) effective amounts of: (a) methionine; (b) a pH buffer; (c) a carbohydrate; (d) a divalent cation; (e) ferric or ferrous ions; (f) glutamine or another nitrogen source; (g) riboflavin; (h) thioctic acid; (i) one or more components selected from leucine, isoleucine, valine, arginine, histidine, tryptophan, and phenylalanine; (j) one or more components selected from adenine, biotin, thiamine, pyridoxal, para-aminobenzoic acid, pantothenate, and nicotinamide; (k) an oxygen source; and (1) one or more components selected from cobalt, copper, boron, manganese, molybdenum, zinc, calcium, and citrate.

In another embodiment, the cell bank, frozen stock, or batch of immunotherapy doses is grown in a defined microbiological media, comprising: (1) between about 0.00123-0.00246 moles of ferric or ferrous ions per liter; and (2) effective amounts of: (a) a pH buffer; (b) a carbohydrate; (c) a divalent cation; (d) methionine; (e) cysteine; (f) glutamine or another nitrogen source; (g) riboflavin; (h) thioctic acid; (i) one or more components selected from leucine, isoleucine, valine, arginine, histidine, tryptophan, and phenylalanine; (j) one or more components selected from adenine, biotin, thiamine, pyridoxal, para-aminobenzoic acid, pantothenate, and nicotinamide; (k) an oxygen source; and (l) one or more components selected from cobalt, copper, boron, manganese, molybdenum, zinc, calcium, and citrate.

In another embodiment, the cell bank, frozen stock, or batch of immunotherapy doses is grown in a defined microbiological media, comprising: (1) between about 1.8-3.6 g/L of glutamine or another nitrogen source; and (2) effective amounts of: (a) a pH buffer; (b) a carbohydrate: (c) a divalent cation; (d) methionine (e) cysteine; (f) ferric or ferrous ions (g) riboflavin (h); thioctic acid; (i) one or more components selected from leucine, isoleucine, valine, arginine, histidine, tryptophan, and phenylalanine; (j) one or more components selected from adenine, biotin, thiamine, pyridoxal, para-aminobenzoic acid, pantothenate, and nicotinamide; (k) an oxygen source; and (l) one or more components selected from cobalt, copper, boron, manganese, molybdenum, zinc, calcium, and citrate.

In another embodiment, the cell bank, frozen stock, or batch of immunotherapy doses is grown in a defined microbiological media, comprising: (1) between about 15 and about 30 mg/L of riboflavin; and (2) effective amounts of: (a) a pH buffer; (b) a carbohydrate; (c) a divalent cation; (d) methionine; (e) cysteine; (f) ferric or ferrous ions; (g) glutamine or another nitrogen source; (h) thioctic acid; (i) one or more components selected from leucine, isoleucine, valine, arginine, histidine, tryptophan, and phenylalanine; (j) one or more components selected from adenine, biotin, thiamine, pyridoxal, para-aminobenzoic acid, pantothenate, and nicotinamide; (k) an oxygen source; and (l) one or more components selected from cobalt, copper, boron, manganese, molybdenum, zinc, calcium, and citrate.

In another embodiment, the cell bank, frozen stock, or batch of immunotherapy doses is grown in a defined microbiological media, comprising (1) between about 0.3 and about 0.6 g/L of thioctic acid; and (2) effective amounts of: (a) a pH buffer; (b) a carbohydrate (c) a divalent cation; (d) methionine (e) cysteine; (f) ferric or ferrous ions; (g) glutamine or another nitrogen source; (h) riboflavin; (i) one or more components selected from leucine, isoleucine, valine, arginine, histidine, tryptophan, and phenylalanine; (j) one or more components selected from adenine, biotin, thiamine, pyridoxal, para-aminobenzoic acid, pantothenate, and nicotinamide; (k) an oxygen source; and (l) one or more components selected from cobalt, copper, boron, manganese, molybdenum, zinc, calcium, and citrate.

In another embodiment, the cell bank, frozen stock, or batch of immunotherapy doses is grown in a defined microbiological media, comprising: (1) between about 0.3 and about 0.6 g/L each of methionine and cysteine; (2) between about 0.00123 and 0.00246 moles of ferric or ferrous ions per liter; (3) between about 1.8 and about 3.6 g/L of glutamine or another nitrogen source; (4) between about 0.3 and about 0.6 g/L of thioctic acid; (5) between about 15 and about 30 mg/L of riboflavin; (6) an oxygen source; and (7) effective amounts of: (a) a pH buffer; (b) a carbohydrate; (c) a divalent cation; (d) one or more components selected from leucine, isoleucine, valine, arginine, histidine, tryptophan, and phenylalanine; (e) one or more components selected from adenine, biotin, thiamine, pyridoxal, para-aminobenzoic acid, pantothenate, and nicotinamide; and (f) one or more components selected from cobalt, copper, boron, manganese, molybdenum, zinc, calcium, and citrate.

In another embodiment, the cell bank, frozen stock, or batch of immunotherapy doses is grown in a defined microbiological media, comprising: (1) between about 0.3 and about 0.6 g/L each of methionine and cysteine; (2) between about 0.00123 and 0.00246 moles of ferric or ferrous ions per liter; (3) between about 1.8 and about 3.6 g/L of glutamine or another nitrogen source; (4) between about 0.3 and about 0.6 g/L of thioctic acid; (5) between about 15 and about 30 mg/L of riboflavin; (6) an oxygen source; and (7) effective amounts of: (a) a pH buffer; (b) a carbohydrate; (c) a divalent cation; (d) leucine; (e) isoleucine; (f) valine; (g) arginine; (h) histidine; (i) tryptophan; (j) phenylalanine; (k) one or more components selected from adenine, biotin, thiamine, pyridoxal, para-aminobenzoic acid, pantothenate, and nicotinamide; and (l) one or more components selected from cobalt, copper, boron, manganese, molybdenum, zinc, calcium, and citrate.

In another embodiment, the cell bank, frozen stock, or batch of immunotherapy doses is grown in a defined microbiological media, comprising (1) between about 0.3 and about 0.6 g/L each of one or more components selected from leucine, isoleucine, valine, arginine, histidine, tryptophan, and phenylalanine; and (2) effective amounts of: (a) a pH buffer; (b) a carbohydrate; (c) a divalent cation; (d) methionine; (e) cysteine; (f) ferric or ferrous ions; (g) glutamine or another nitrogen source; (h) riboflavin; (i) thioctic acid; (j) one or more components selected from adenine, biotin, thiamine, pyridoxal, para-aminobenzoic acid, pantothenate, and nicotinamide; (k) an oxygen source; and (l) one or more components selected from cobalt, copper, boron, manganese, molybdenum, zinc, calcium, and citrate.

In another embodiment, the cell bank, frozen stock, or batch of immunotherapy doses is grown in a defined microbiological media, comprising (1) between about 0.3 and about 0.6 g/L each of leucine, isoleucine, valine, arginine, histidine, tryptophan, and phenylalanine; and (2) effective amounts of: (a) a pH buffer; (b) a carbohydrate; (c) a divalent cation; (d) methionine; (e) cysteine; (f) ferric or ferrous ions; (g) glutamine or another nitrogen source; (h) riboflavin; (i) thioctic acid; (j) one or more components selected from adenine, biotin, thiamine, pyridoxal, para-aminobenzoic acid, pantothenate, and nicotinamide; (k) an oxygen source; and (l) one or more components selected from cobalt, copper, boron, manganese, molybdenum, zinc, calcium, and citrate.

In another embodiment, the cell bank, frozen stock, or batch of immunotherapy doses is grown in a defined microbiological media, comprising (1) between about 0.2 and about 0.75 of one or more components selected from biotin and adenine; and (2) effective amounts of: (a) a pH buffer; (b) a carbohydrate; (c) a divalent cation; (d) methionine; (e) cysteine; (f) ferric or ferrous ions; (g) glutamine or another nitrogen source; (h) riboflavin; (i) thioctic acid; (j) one or more components selected from leucine, isoleucine, valine, arginine, histidine, tryptophan, and phenylalanine; (k) an oxygen source; (l) one or more components selected from thiamine, pyridoxal, para-aminobenzoic acid, pantothenate, and nicotinamide; and (m) one or more components selected from cobalt, copper, boron, manganese, molybdenum, zinc, calcium, and citrate.

In another embodiment, the cell bank, frozen stock, or batch of immunotherapy doses is grown in a defined microbiological media, comprising (1) between about 3 and about 6 mg/L each of one or more components selected from thiamine, pyridoxal, para-aminobenzoic acid, pantothenate, and nicotinamide; and (2) effective amounts of: (a) a pH buffer; (b) a carbohydrate; (c) a divalent cation; (d) methionine; (e) cysteine; (f) ferric or ferrous ions; (g) glutamine or another nitrogen source; (h) riboflavin; (i) thioctic acid; (j) one or more components selected from leucine, isoleucine, valine, arginine, histidine, tryptophan, and phenylalanine; (k) biotin; (l) adenine; (l) an oxygen source; and (m) one or more components selected from cobalt, copper, boron, manganese, molybdenum, zinc, calcium, and citrate.

In another embodiment, the cell bank, frozen stock, or batch of immunotherapy doses is grown in a defined microbiological media, comprising: (1) between about 0.2 and about 0.75 mg/L each of one or more components selected from biotin and adenine; (2) between about 3 and about 6 mg/L each of one or more components selected from thiamine, pyridoxal, para-aminobenzoic acid, pantothenate, and nicotinamide; and (3) effective amounts of: (a) a pH buffer; (b) a carbohydrate; (c) a divalent cation; (d) methionine; (e) cysteine; (f) ferric or ferrous ions; (g) glutamine or another nitrogen source; (h) riboflavin; (i) thioctic acid; (j) one or more components selected from leucine, isoleucine, valine, arginine, histidine, tryptophan, and phenylalanine; (k) an oxygen source; and (l) one or more components selected from cobalt, copper, boron, manganese, molybdenum, zinc, calcium, and citrate.

In another embodiment, the cell bank, frozen stock, or batch of immunotherapy doses is grown in a defined microbiological media, comprising: (1) between about 0.005 and about 0.02 g/L each of one or more components selected from cobalt, copper, boron, manganese, molybdenum, zinc, and calcium; and (2) effective amounts of: (a) a pH buffer; (b) a carbohydrate; (c) a divalent cation; (d) methionine; (e) cysteine; (f) ferric or ferrous ions; (g) glutamine or another nitrogen source; (h) riboflavin; (i) thioctic acid; (j) one or more components selected from leucine, isoleucine, valine, arginine, histidine, tryptophan, and phenylalanine; (k) an oxygen source; and (l) one or more components selected from adenine, biotin, thiamine, pyridoxal, para-aminobenzoic acid, pantothenate, and nicotinamide.

In another embodiment, the cell bank, frozen stock, or batch of immunotherapy doses is grown in a defined microbiological media, comprising: (1) between about 0.4 and about 1 g/L of citrate; and (2) effective amounts of: (a) a pH buffer; (b) a carbohydrate; (c) a divalent cation; (d) methionine; (e) cysteine; (f) ferric or ferrous ions; (g) glutamine or another nitrogen source; (h) riboflavin; (i) thioctic acid; (j) one or more components selected from leucine, isoleucine, valine, arginine, histidine, tryptophan, and phenylalanine; (k) one or more components selected from cobalt, copper, boron, manganese, molybdenum, zinc, and calcium; (k) an oxygen source; and (m) one or more components selected from adenine, biotin, thiamine, pyridoxal, para-aminobenzoic acid, pantothenate, and nicotinamide.

In another embodiment, the cell bank, frozen stock, or batch of immunotherapy doses is grown in a defined microbiological media, comprising: (1) between about 0.3 and about 0.6 g/L each of methionine and cysteine; (2) between about 0.00123 and 0.00246 moles of ferric or ferrous ions per liter; (3) between about 1.8 and about 3.6 g/L of glutamine or another nitrogen source; (4) between about 0.3 and about 0.6 g/L of thioctic acid; (5) between about 15 and about 30 mg/L of riboflavin; (6) between about 0.3 and about 0.6 g/L each of one or more components selected from leucine, isoleucine, valine, arginine, histidine, tryptophan, and phenylalanine; (7) between about 0.2 and about 0.75 mg/L each of one or more components selected from biotin and adenine; (8) between about 3 and about 6 mg/L each of one or more components selected from thiamine, pyridoxal, para-aminobenzoic acid, pantothenate, and nicotinamide; (9) between about 0.005 and about 0.02 g/L each of one or more components selected from cobalt, copper, boron, manganese, molybdenum, zinc, and calcium; (10) between about 0.4 and about 1 g/L of citrate; and (11) and effective amounts of: (a) a pH buffer; (b) a carbohydrate; and (c) a divalent cation.

In another embodiment, the cell bank, frozen stock, or batch of immunotherapy doses is grown in a defined microbiological media, comprising: (1) between about 0.3 and about 0.6 g/L each of methionine and cysteine; (2) between about 0.00123 and 0.00246 moles of ferric or ferrous ions per liter; (3) between about 1.8 and about 3.6 g/L of glutamine or another nitrogen source; (4) between about 0.3 and about 0.6 g/L of thioctic acid; (5) between about 15 and about 30 mg/L of riboflavin; (6) between about 0.3 and about 0.6 g/L each of leucine, isoleucine, valine, arginine, histidine, tryptophan, and phenylalanine; (7) between about 0.2 and about 0.75 mg/L each of one or more components selected from biotin and adenine; (8) between about 3 and about 6 mg/L each of one or more components selected from thiamine, pyridoxal, para-aminobenzoic acid, pantothenate, and nicotinamide; (9) between about 0.005 and about 0.02 g/L each of one or more components selected from cobalt, copper, boron, manganese, molybdenum, zinc, and calcium; (10) between about 0.4 and about 1 g/L of citrate; and (11) and effective amounts of: (a) a pH buffer; (b) a carbohydrate; and (c) a divalent cation.

In another embodiment, the cell bank, frozen stock, or batch of immunotherapy doses is grown in a defined microbiological media, comprising: (1) between about 0.3 and about 0.6 g/L each of methionine and cysteine; (2) between about 0.00123 and 0.00246 moles of ferric or ferrous ions per liter; (3) between about 1.8 and about 3.6 g/L of glutamine or another nitrogen source; (4) between about 0.3 and about 0.6 g/L of thioctic acid; (5) between about 15 and about 30 mg/L of riboflavin; (6) between about 0.3 and about 0.6 g/L each of leucine, isoleucine, valine, arginine, histidine, tryptophan, and phenylalanine; (7) between about 0.2 and about 0.75 mg/L each of biotin and adenine; (8) between about 3 and about 6 mg/L each of thiamine, pyridoxal, para-aminobenzoic acid, pantothenate, and nicotinamide; (9) between about 0.005 and about 0.02 g/L each of one or more components selected from cobalt, copper, boron, manganese, molybdenum, zinc, and calcium; (10) between about 0.4 and about 1 g/L of citrate; and (11) and effective amounts of: (a) a pH buffer; (b) a carbohydrate; and (c) a divalent cation.

In another embodiment, a fermentation media disclosed herein comprises an aqueous solvent. In another embodiment, the aqueous solvent is water. In another embodiment, the solvent is Water for Injection (WFI). In another embodiment, the aqueous solvent is any other aqueous solvent known in the art.

In one embodiment, a fermentation media disclosed herein comprises any 2 of the components listed in Table 3. In another embodiment, a fermentation media disclosed herein comprises any 3 of the components listed in Table 3. In another embodiment, a fermentation media disclosed herein comprises any 4 of the components listed in Table 3. In another embodiment, a fermentation media disclosed herein comprises any 5 of the components listed in Table 3. In another embodiment, a fermentation media disclosed herein comprises any 6 of the components listed in Table 3. In another embodiment, a fermentation media disclosed herein comprises all of the components listed in Table 3. In one embodiment, a buffer media disclosed herein comprises any 2 of the components listed in Table 4. In another embodiment, a buffer media disclosed herein comprises any 3 of the components listed in Table 4. In another embodiment, a buffer media disclosed herein comprises any 4 of the components listed in Table 4. In another embodiment, a buffer media disclosed herein comprises any 5 of the components listed in Table 4. In another embodiment, a buffer media disclosed herein comprises all of the components listed in Table 4.

In another embodiment, a defined microbiological media of the present disclosure further comprises an aqueous solvent. In another embodiment, the aqueous solvent is water. In another embodiment, the aqueous solvent is any other aqueous solvent known in the art.

The carbohydrate utilized in methods and compositions of the present disclosure is, in another embodiment, glucose. In another embodiment, the carbohydrate is fructose.

In another embodiment, the carbohydrate is sucrose. In another embodiment, the carbohydrate is maltose. In another embodiment, the carbohydrate is lactose. In another embodiment, the carbohydrate is fructose. In another embodiment, the carbohydrate is mannose. In another embodiment, the carbohydrate is cellobiose. In another embodiment, the carbohydrate is trehalose. In another embodiment, the carbohydrate is maltose. In another embodiment, the carbohydrate is glycerol. In another embodiment, the carbohydrate is glucosamine. In another embodiment, the carbohydrate is N-acetylglucosamine. In another embodiment, the carbohydrate is N-acetylmuramic acid. In another embodiment, the carbohydrate is any other carbohydrate that can be utilized by Listeria.

In another embodiment, the amount of a carbohydrate present in a defined microbiological media of methods and compositions of the present disclosure is between about 12-18 grams/liter (g/L). In another embodiment, the amount is 15 g/L. In another embodiment, the amount is 10 g/L. In another embodiment, the amount is 9 g/L. In another embodiment, the amount is 11 g/L. In another embodiment, the amount is 12 g/L. In another embodiment, the amount is 13 g/L. In another embodiment, the amount is 14 g/L. In another embodiment, the amount is 16 g/L. In another embodiment, the amount is 17 g/L. In another embodiment, the amount is 18 g/L. In another embodiment, the amount is 19 g/L. In another embodiment, the amount is 20 g/L. In another embodiment, the amount is more than 20 g/L.

In another embodiment, the amount is 9-15 g/L. In another embodiment, the amount is 10-15 g/L. In another embodiment, the amount is 11-15 g/L. In another embodiment, the amount is 12-16 g/L. In another embodiment, the amount is 13-17 g/L. In another embodiment, the amount is 14-18 g/L. In another embodiment, the amount is 16-19 g/L. In another embodiment, the amount is 17-20 g/L. In another embodiment, the amount is 10-20 g/L. In another embodiment, the amount is 12-20 g/L. In another embodiment, the amount is 15-20 g/L.

In another embodiment, the total amount of carbohydrate in the media is one of the above amounts. In another embodiment, the amount of one of the carbohydrates in the media is one of the above amounts. In another embodiment, the amount of each of the carbohydrates in the media is one of the above amounts.

The cobalt present in defined microbiological media of methods and compositions of the present disclosure is, in another embodiment, present as a cobalt ion. In another embodiment, the cobalt is present as a cobalt salt. In another embodiment, the salt is cobalt chloride. In another embodiment, the salt is any other cobalt salt known in the art. In another embodiment, the cobalt is present as any other form of cobalt known in the art.

In another embodiment, the cobalt salt is a hydrate (e.g. cobalt chloride hexahydrate). In another embodiment, the cobalt salt is anhydrous. In another embodiment, the cobalt salt is any other form of a cobalt salt known in the art.

A hydrate of a component of a defined media of methods and compositions of the present disclosure is, in another embodiment, a monohydrate. In another embodiment, the hydrate is a dihydrate. In another embodiment, the hydrate is a trihydrate. In another embodiment, the hydrate is a tetrahydrate. In another embodiment, the hydrate is a pentahydrate. In another embodiment, the hydrate is a hexahydrate. In another embodiment, the hydrate is a heptahydrate. In another embodiment, the hydrate is any other hydrate known in the art.

The copper present in defined microbiological media of the methods and compositions disclosed herein is, in another embodiment, present as a copper ion. In another embodiment, the copper ion is a copper (I) ion. In another embodiment, the copper ion is a copper (II) ion. In another embodiment, the copper ion is a copper (III) ion.

In another embodiment, the copper is present as a copper salt. In another embodiment, the salt is copper chloride. In another embodiment, the salt is any other copper salt known in the art. In another embodiment, the copper is present as any other form of copper known in the art.

In another embodiment, the copper salt is a hydrate (e.g. copper chloride dihydrate). In another embodiment, the copper salt is anhydrous. In another embodiment, the copper salt is any other form of a copper salt known in the art.

The boron present in defined microbiological media of methods and compositions of the present disclosure is, in another embodiment, present as a borate ion. In another embodiment, the boron is present as a borate acid (e.g. boric acid, H₃BO₃). In another embodiment, the boron is present as any other form of boron known in the art.

In another embodiment, the borate salt or borate acid is a hydrate. In another embodiment, the borate salt or borate acid is anhydrous. In another embodiment, the borate salt or borate acid is any other form of a borate salt or borate acid known in the art.

The manganese present in defined microbiological media of methods and compositions of the present disclosure is, in another embodiment, present as a manganese ion. In another embodiment, the manganese is present as a manganese salt. In another embodiment, the salt is manganese sulfate. In another embodiment, the salt is any other manganese salt known in the art. In another embodiment, the manganese is present as any other form of manganese known in the art.

In another embodiment, the manganese salt is a hydrate (e.g. manganese sulfate monohydrate). In another embodiment, the manganese salt is anhydrous. In another embodiment, the manganese salt is any other form of a manganese salt known in the art.

The molybdenum present in defined microbiological media of methods and compositions of the present disclosure is, in another embodiment, present as a molybdate ion. In another embodiment, the molybdenum is present as a molybdate salt. In another embodiment, the salt is sodium molybdate. In another embodiment, the salt is any other molybdate salt known in the art. In another embodiment, the molybdenum is present as any other form of molybdenum known in the art.

In another embodiment, the molybdate salt is a hydrate (e.g. sodium molybdate dihydrate). In another embodiment, the molybdate salt is anhydrous. In another embodiment, the molybdate salt is any other form of a molybdate salt known in the art.

In one embodiment, when zinc is present in a defined microbiological media of methods and compositions of the present disclosure it is, in another embodiment, present as a zinc ion. In another embodiment, the zinc is present as a zinc salt. In another embodiment, the salt is zinc chloride. In another embodiment, the salt is any other zinc salt known in the art. In another embodiment, the zinc is present as any other form of zinc known in the art.

In another embodiment, the zinc salt is a hydrate (e.g. zinc chloride heptahydrate). In another embodiment, the zinc salt is anhydrous. In another embodiment, the zinc salt is any other form of a zinc salt known in the art.

In one embodiment, when iron is present in defined microbiological media of methods and compositions of the present disclosure it is present as a ferric ion. In another embodiment, the iron is present as a ferrous ion. In another embodiment, the iron is present as a ferric salt. In another embodiment, the iron is present as a ferrous salt. In another embodiment, the salt is ferric sulfate. In another embodiment, the salt is ferric citrate. In another embodiment, the salt is any other ferric salt known in the art. In another embodiment, the salt is any other ferrous salt known in the art. In another embodiment, the iron is present as any other form of iron known in the art.

In another embodiment, the ferric or ferrous salt is a hydrate (e.g. ferric sulfate monohydrate). In another embodiment, the ferric or ferrous salt is anhydrous. In another embodiment, the ferric or ferrous salt is any other form of a ferric or ferrous salt known in the art.

The calcium present in defined microbiological media of methods and compositions of the present disclosure is, in another embodiment, present as a calcium ion. In another embodiment, the calcium is present as a calcium salt. In another embodiment, the salt is calcium chloride. In another embodiment, the salt is any other calcium salt known in the art. In another embodiment, the calcium is present as any other form of calcium known in the art.

In another embodiment, the calcium salt is a hydrate (e.g. calcium chloride dihydrate). In another embodiment, the calcium salt is anhydrous. In another embodiment, the calcium salt is any other form of a calcium salt known in the art.

The citrate present in defined microbiological media of methods and compositions of the present disclosure is, in another embodiment, present as a citrate ion. In another embodiment, the citrate is present as a citrate salt. In another embodiment, the citrate is present as a citrate acid (e.g. citric acid). In another embodiment, the citrate is present as both ferric citrate and citric acid. In another embodiment, the citrate is present as any other form of citrate known in the art.

In another embodiment, the citrate salt or citrate acid is a hydrate. In another embodiment, the citrate salt or citrate acid is anhydrous. In another embodiment, the citrate salt or citrate acid is any other form of a citrate salt or citrate acid known in the art.

The cobalt present in defined microbiological media of methods and compositions of the present disclosure is, in another embodiment, present in an amount of 0.02 g/L. In another embodiment, the amount is about 0.02 g/L. In another embodiment, the amount is 0.003 g/L. In another embodiment, the amount is 0.005 g/L. In another embodiment, the amount is 0.007 g/L. In another embodiment, the amount is 0.01 g/L. In another embodiment, the amount is 0.015 g/L. In another embodiment, the amount is 0.025 g/L. In another embodiment, the amount is 0.03 g/L. In another embodiment, the amount is 0.003-0.006 g/L. In another embodiment, the amount is 0.005-0.01 g/L. In another embodiment, the amount is 0.01-0.02 g/L. In another embodiment, the amount is 0.02-0.04 g/L. In another embodiment, the amount is 0.03-0.06 g/L.

In another embodiment, the cobalt is present in an amount that is the molar equivalent of 0.02 g/L of cobalt chloride hexahydrate. In another embodiment, the amount of cobalt present is the molar equivalent of about 0.02 g/L of cobalt chloride hexahydrate. In another embodiment, the amount of cobalt present is the molar equivalent of another of the above amounts or ranges of cobalt chloride hexahydrate.

The copper present in defined microbiological media of methods and compositions of the present disclosure is, in another embodiment, present in an amount of 0.019 g/L. In another embodiment, the amount is about 0.019 g/L. In other embodiments, the amount is any of the amounts or ranges listed above for cobalt.

In another embodiment, the copper is present in an amount that is the molar equivalent of 0.019 g/L of copper chloride dihydrate. In another embodiment, the amount of copper present is the molar equivalent of about 0.019 g/L of copper chloride dihydrate. In another embodiment, the amount of copper present is the molar equivalent of copper chloride dihydrate in any of the amounts or ranges listed above for cobalt.

The borate present in defined microbiological media of methods and compositions of the present disclosure is, in another embodiment, present in an amount of 0.016 g/L. In another embodiment, the amount is about 0.016 g/L. In other embodiments, the amount is any of the amounts or ranges listed above for cobalt.

In another embodiment, the borate is present in an amount that is the molar equivalent of 0.016 g/L of boric acid. In another embodiment, the amount of borate present is the molar equivalent of about 0.016 g/L of boric acid. In another embodiment, the amount of borate present is the molar equivalent of boric acid in any of the amounts or ranges listed above for cobalt.

The manganese present in defined microbiological media of methods and compositions of the present disclosure is, in another embodiment, present in an amount of 0.016 g/L. In another embodiment, the amount is about 0.016 g/L. In other embodiments, the amount is any of the amounts or ranges listed above for cobalt.

In another embodiment, the manganese is present in an amount that is the molar equivalent of 0.016 g/L of manganese sulfate monohydrate. In another embodiment, the amount of manganese present is the molar equivalent of about 0.016 g/L of manganese sulfate monohydrate. In another embodiment, the amount of manganese present is the molar equivalent of manganese sulfate monohydrate in any of the amounts or ranges listed above for cobalt.

The molybdenum present in defined microbiological media of methods and compositions of the present disclosure is, in another embodiment, present in an amount of 0.02 g/L. In another embodiment, the amount is about 0.02 g/L. In other embodiments, the amount is any of the amounts or ranges listed above for cobalt.

In another embodiment, the molybdenum is present in an amount that is the molar equivalent of 0.2 g/L of sodium molybdate dihydrate. In another embodiment, the amount of molybdenum present is the molar equivalent of about 0.02 g/L of sodium molybdate dihydrate. In another embodiment, the amount of molybdenum present is the molar equivalent of sodium molybdate dihydrate in any of the amounts or ranges listed above for cobalt.

The zinc present in defined microbiological media of methods and compositions of the present disclosure is, in another embodiment, present in an amount of 0.02 g/L. In another embodiment, the amount is about 0.02 g/L. In other embodiments, the amount is any of the amounts or ranges listed above for cobalt.

In another embodiment, the zinc is present in an amount that is the molar equivalent of 0.02 g/L of zinc chloride heptahydrate. In another embodiment, the amount of zinc present is the molar equivalent of about 0.02 g/L of zinc chloride heptahydrate. In another embodiment, the amount of zinc present is the molar equivalent of zinc chloride heptahydrate in any of the amounts or ranges listed above for cobalt.

In another embodiment, ferric sulfate or a related compound is present in defined microbiological media of methods and compositions of the present disclosure. In another embodiment, the ferric sulfate or related compound is present in an amount of 0.01 g/L. In another embodiment, the amount is about 0.01 g/L. In other embodiments, the amount is any of the amounts or ranges listed above for cobalt.

In another embodiment, the iron is present in an amount that is the molar equivalent of 0.01 g/L of ferric sulfate. In another embodiment, the amount of iron present is the molar equivalent of about 0.01 g/L of ferric sulfate. In another embodiment, the amount of iron present is the molar equivalent of ferric sulfate in any of the amounts or ranges listed above for cobalt.

The calcium present in defined microbiological media of methods and compositions of the present disclosure is, in another embodiment, present in an amount of 0.01 g/L. In another embodiment, the amount is about 0.01 g/L. In other embodiments, the amount is any of the amounts or ranges listed above for cobalt.

In another embodiment, the calcium is present in an amount that is the molar equivalent of 0.01 g/L of calcium chloride dihydrate. In another embodiment, the amount of calcium present is the molar equivalent of about 0.01 g/L of calcium chloride dihydrate. In another embodiment, the amount of calcium present is the molar equivalent of calcium chloride dihydrate in any of the amounts or ranges listed above for cobalt.

The citrate present in defined microbiological media of methods and compositions of the present disclosure is, in another embodiment, present in an amount of 0.9 g/L. In another embodiment, the amount is 0.6 g/L in the form of citric acid. In another embodiment, the amount is 0.4 g/L in the form of ferric citrate. In another embodiment, the amount is 0.6 g/L in the form of citric acid and 0.4 g/L in the form of ferric citrate. In another embodiment, the amount is about 0.6 g/L. In another embodiment, the amount is 0.1 g/L. In another embodiment, the amount is 0.2 g/L. In another embodiment, the amount is 0.3 g/L. In another embodiment, the amount is 0.4 g/L. In another embodiment, the amount is 0.5 g/L. In another embodiment, the amount is 0.7 g/L. In another embodiment, the amount is 0.8 g/L. In another embodiment, the amount is 1 g/L. In another embodiment, the amount is more than 1 g/L.

In another embodiment, the citrate is present in an amount that is the molar equivalent of 0.6 g/L of citric acid. In another embodiment, the amount of citrate present is the molar equivalent of about 0.6 g/L of citric acid. In another embodiment, the amount of citrate present is the molar equivalent of about 0.4 g/L of ferric citrate. In another embodiment, the amount of citrate present is the molar equivalent of 0.4 g/L of ferric citrate. In another embodiment, the amount of citrate present is the molar equivalent of 0.6 g/L of citric acid and 0.4 g/L of ferric citrate. In another embodiment, the amount of citrate present is the about molar equivalent of 0.6 g/L of citric acid and 0.4 g/L of ferric citrate. In another embodiment, the amount of citrate present is the molar equivalent of citric acid in any of the amounts or ranges listed above for citrate.

One or more of the adenine, biotin, thiamine, pyridoxal, para-aminobenzoic acid, pantothenate, and nicotinamide present in defined microbiological media of methods and compositions of the present disclosure are, in another embodiment, present as the free compound. In another embodiment, one of the above compounds is present as a salt thereof. In another embodiment, one of the above compounds is present as a derivative thereof. In another embodiment, one of the above compounds is present as a hydrate thereof. In other embodiments, the salt, derivative, or hydrate can be any salt, derivative, or hydrate known in the art.

The thiamine (vitamin B1) present in defined microbiological media of methods and compositions of the present disclosure is, in another embodiment, present in the form of thiamine HCl. In another embodiment, the thiamine is present as any other salt, derivative, or hydrate of thiamine known in the art. In another embodiment, another form of vitamin B1 is substituted for thiamine.

In another embodiment, the thiamine is present in an amount of 4 mg/L. In another embodiment, the amount is about 0.5 mg/L. In another embodiment, the amount is 0.7 mg/L. In another embodiment, the amount is 1 mg/L. In another embodiment, the amount is 1.5 mg/L. In another embodiment, the amount is 2 mg/L. In another embodiment, the amount is 3 mg/L. In another embodiment, the amount is 5 mg/L. In another embodiment, the amount is 6 mg/L. In another embodiment, the amount is 8 mg/L. In another embodiment, the amount is more than 8 mg/L. In another embodiment, the thiamine is present in an amount that is the molar equivalent of 4 mg/L of thiamine HCl. In another embodiment, the thiamine is present in an amount that is the molar equivalent of thiamine HCl in one of the above amounts.

The pyridoxal (vitamin B6) present in defined microbiological media of methods and compositions of the present disclosure is, in another embodiment, present in the form of pyridoxal HCl. In another embodiment, the pyridoxal is present as any other salt, derivative, or hydrate of pyridoxal known in the art. In another embodiment, another form of vitamin B6 is substituted for pyridoxal.

In another embodiment, the pyridoxal is present in an amount of 4 mg/L. In another embodiment, the amount is any of the amounts or ranges listed above for thiamine. In another embodiment, the amount of pyridoxal present is the molar equivalent of about 4 mg/L of pyridoxal HCl. In another embodiment, the amount of pyridoxal present is the molar equivalent of pyridoxal HCl in any of the amounts or ranges listed above for thiamine.

The adenine (vitamin B4) present in defined microbiological media of methods and compositions of the present disclosure is, in another embodiment, present in the form of free adenine. In another embodiment, the adenine is present as any other salt, derivative, or hydrate of adenine known in the art. In another embodiment, another form of vitamin B4 is substituted for adenine.

In another embodiment, the adenine is present in an amount of 0.25 mg/L. In another embodiment, the amount is any of the amounts or ranges listed above for cobalt. In another embodiment, the amount of adenine present is the molar equivalent of about 0.25 mg/L of free adenine. In another embodiment, the amount of adenine present is the molar equivalent of free adenine in any of the amounts or ranges listed above for cobalt.

The biotin (vitamin B7) present in defined microbiological media of methods and compositions of the present disclosure is, in another embodiment, present in the form of free biotin. In another embodiment, the biotin is present as any other salt, derivative, or hydrate of biotin known in the art. In another embodiment, another form of vitamin B7 is substituted for biotin.

In another embodiment, the biotin is present in an amount of 2 mg/L. In another embodiment, the amount is any of the amounts or ranges listed above for thiamine. In another embodiment, the amount of biotin present is the molar equivalent of about 2 mg/L of free biotin. In another embodiment, the amount of biotin present is the molar equivalent of free biotin in any of the amounts or ranges listed above for thiamine.

The para-aminobenzoic acid (vitamin B-x) present in defined microbiological media of methods and compositions of the present disclosure is, in another embodiment, present in the form of free para-aminobenzoic acid. In another embodiment, the para-aminobenzoic acid is present as any other salt, derivative, or hydrate of para-aminobenzoic acid known in the art. In another embodiment, another form of vitamin B-x is substituted for para-aminobenzoic acid.

In another embodiment, the para-aminobenzoic acid is present in an amount of 4 mg/L. In another embodiment, the amount is any of the amounts or ranges listed above for thiamine. In another embodiment, the amount of para-aminobenzoic acid present is the molar equivalent of about 4 mg/L of free para-aminobenzoic acid. In another embodiment, the amount of para-aminobenzoic acid present is the molar equivalent of free para-aminobenzoic acid in any of the amounts or ranges listed above for thiamine.

The pantothenate (vitamin B5) present in defined microbiological media of methods and compositions of the present disclosure is, in another embodiment, present in the form of calcium pantothenate. In another embodiment, the pantothenate is present as any other salt, derivative, or hydrate of pantothenate known in the art. In another embodiment, another form of vitamin B5 is substituted for pantothenate.

In another embodiment, the pantothenate is present in an amount of 4 mg/L. In another embodiment, the amount is any of the amounts or ranges listed above for thiamine. In another embodiment, the amount of pantothenate present is the molar equivalent of about 4 mg/L of calcium pantothenate. In another embodiment, the amount of pantothenate present is the molar equivalent of calcium pantothenate in any of the amounts or ranges listed above for thiamine.

The nicotinamide (vitamin B3) present in defined microbiological media of methods and compositions of the present disclosure is, in another embodiment, present in the form of free nicotinamide. In another embodiment, the nicotinamide is present as any other salt, derivative, or hydrate of nicotinamide known in the art. In another embodiment, another form of vitamin B3 is substituted for nicotinamide.

In another embodiment, the nicotinamide is present in an amount of 4 mg/L. In another embodiment, the amount is any of the amounts or ranges listed above for thiamine. In another embodiment, the amount of nicotinamide present is the molar equivalent of about 4 mg/L of free nicotinamide. In another embodiment, the amount of nicotinamide present is the molar equivalent of free nicotinamide in any of the amounts or ranges listed above for thiamine.

One or more of the leucine, isoleucine, valine, arginine, histidine, tryptophan, and phenylalanine present in defined microbiological media of methods and compositions of the present disclosure are, in another embodiment, present as free amino acids. In another embodiment, one of the above compounds is present as a salt thereof. In another embodiment, one of the above compounds is present as a derivative thereof. In another embodiment, one of the above compounds is present as a hydrate thereof. In other embodiments, the salt, derivative, or hydrate can be any salt, derivative, or hydrate known in the art. Each of the above forms of adenine, biotin, thiamine, pyridoxal, para-aminobenzoic acid, pantothenate, and nicotinamide represents a separate embodiment of the present disclosure.

In another embodiment, one or more of the leucine, isoleucine, valine, arginine, histidine, tryptophan, and phenylalanine is present in an amount of 0.4 g/L. In another embodiment, the amount is about 0.05 g/L. In another embodiment, the amount is 0.07 g/L. In another embodiment, the amount is 0.1 g/L. In another embodiment, the amount is 0.15 g/L. In another embodiment, the amount is 0.2 g/L. In another embodiment, the amount is 0.3 g/L. In another embodiment, the amount is 0.5 g/L. In another embodiment, the amount is 0.6 g/L. In another embodiment, the amount is 0.8 g/L. In another embodiment, the amount is more than 0.8 g/L. In another embodiment, one or more of these AA is present in an amount that is the molar equivalent of 0.4 g/L of the free AA. In another embodiment, the amount is the molar equivalent of thiamine the free AA in one of the above amounts.

In another embodiment, a defined media of methods and compositions of the present disclosure contains two of the amino acids (AA) selected from the following leucine, isoleucine, valine, arginine, histidine, tryptophan, and phenylalanine. In another embodiment, the defined media contains 3 of these AA. In another embodiment, the media contains 4 of these AA. In another embodiment, the media contains 3 of these AA. In another embodiment, the media contains 5 of these AA. In another embodiment, the media contains 6 of these AA. In another embodiment, the media contains all of these AA. In another embodiment, the media contains at least 2 of these AA. In another embodiment, the media contains at least 3 of these AA. In another embodiment, the media contains at least 4 of these AA. In another embodiment, the media contains at least 5 of these AA. In another embodiment, the media contains at least 6 of these AA.

In another embodiment, a defined media of methods and compositions of the present disclosure comprises 2 of the following vitamins adenine, biotin, thiamine, pyridoxal, para-aminobenzoic acid, pantothenate, and nicotinamide. In another embodiment, the defined media comprises 3 of these vitamins. In another embodiment, the media comprises 4 of these vitamins. In another embodiment, the media comprises 3 of these vitamins. In another embodiment, the media comprises 5 of these vitamins. In another embodiment, the media comprises 6 of these vitamins. In another embodiment, the media comprises all of these vitamins. In another embodiment, the media comprises at least 2 of these vitamins. In another embodiment, the media comprises at least 3 of these vitamins. In another embodiment, the media comprises at least 4 of these vitamins. In another embodiment, the media comprises at least 5 of these vitamins. In another embodiment, the media comprises at least 6 of these vitamins.

In another embodiment, a defined media of methods and compositions of the present disclosure comprises 2 of the following trace elements: cobalt, copper, boron, manganese, molybdenum, zinc, iron, calcium, and citrate. In another embodiment, the defined media comprises 3 of these trace elements. In another embodiment, the media comprises 4 of these trace elements. In another embodiment, the media comprises 3 of these trace elements. In another embodiment, the media comprises 5 of these trace elements. In another embodiment, the media comprises 6 of these trace elements. In another embodiment, the media comprises 7 of these trace elements. In another embodiment, the media comprises 7 of these trace elements. In another embodiment, the media comprises all of these trace elements. In another embodiment, the media comprises at least 2 of these trace elements. In another embodiment, the media comprises at least 3 of these trace elements. In another embodiment, the media comprises at least 4 of these trace elements. In another embodiment, the media comprises at least 5 of these trace elements. In another embodiment, the media comprises at least 6 of these trace elements. In another embodiment, the media comprises at least 7 of these trace elements. In another embodiment, the media comprises at least 8 of these trace elements.

In another embodiment, a defined media of methods and compositions of the present disclosure comprises more than 1 component from 2 of the above classes of components; e.g more than one of the AA listed, and more than one of the vitamins listed in the third section. In another embodiment, the media comprises more than 2 components from 2 of the above classes of components; e.g. more than 2 of the AA listed in the second section of Table 3, and more than 2 of the trace elements listed in the fourth section. In another embodiment, the media comprises more than 3 components from 2 of the above classes. In another embodiment, the media comprises more than 4 components from 2 of the above classes. In another embodiment, the media comprises more than 5 components from 2 of the above classes. In another embodiment, the media comprises more than 6 components from 2 of the above classes. In another embodiment, the media comprises all of the components from 2 of the above classes.

In another embodiment, a media of methods and compositions of the present disclosure comprises more than 1 component from all of the above classes of components (e.g. more than 1 component each from AA, vitamins and trace elements). In another embodiment, the media comprises more than 2 components from all of the above classes of components. In another embodiment, the media comprises more than 3 components from all of the above classes. In another embodiment, the media comprises more than 4 components from all of the above classes. In another embodiment, the media comprises more than all components from 2 of the above classes. In another embodiment, the media comprises more than 6 components from all of the above classes. In another embodiment, the media comprises all of the components from all of the above classes.

In another embodiment, the media comprises any other combination of numbers of components from each of the above classes; e.g. 2 AA, 2 vitamins, and 3 trace elements; 3 AA, 3 vitamins, and 2 trace elements; 2 AA, 3 vitamins, and all of the trace elements, etc.

In another embodiment, a media of methods and compositions of the present disclosure consists of one of the above recipes, mixtures of components, lists of components in specified amounts, or combinations of numbers of components from each of the above classes.

The divalent cation present in defined microbiological media of methods and compositions of the present disclosure is, in another embodiment, Mg. In another embodiment, the divalent cation is Ca. In another embodiment, the divalent cation is any other divalent cation known in the art. Mg can, in other embodiments, be present in any form of Mg known in the art, e.g. MgSO₄. In another embodiment, the divalent cation is present in an amount that is the molar equivalent of about 0.41 g/mL. In other embodiments, the divalent cation is present in another effective amount, as known to those skilled in the art.

In another embodiment, a nitrogen source other than glutamine is utilized in defined media disclosed herein. In another embodiment, nitrogen gas is utilized in defined media disclosed herein. In another embodiment, oxygen gas is utilized in defined media of the present disclosure. In another embodiment, both nitrogen and oxygen gases are utilized in a defined media disclosed herein. In another embodiment, the nitrogen source is another AA. In another embodiment, the nitrogen source is another source of peptides or proteins (e.g. casitone or casamino acids). In another embodiment, the nitrogen source is ammonium chloride. In another embodiment, the nitrogen source is ammonium nitrate. In another embodiment, the nitrogen source is ammonium sulfate. In another embodiment, the nitrogen source is another ammonium salt. In another embodiment, the nitrogen source is any other nitrogen source known in the art.

In another embodiment, a defined microbiological media of methods and compositions of the present disclosure does not contain a component derived from an animal source. In another embodiment, the defined microbiological media does not contain an animal-derived component of incompletely defined composition (e.g. yeast extract, bacto-tryptone, etc.).

In another embodiment, “defined microbiological media” refers to a media whose components are known. In another embodiment, the term refers to a media that does not contain a component derived from an animal source. In another embodiment, the term refers to a media whose components have been chemically characterized.

In another embodiment, a defined media of methods and compositions of the present disclosure supports growth of the Listeria strain to about 1.1×10¹⁰ CFU/mL (e.g. when grown in flasks;). In another embodiment, the defined media supports growth to about 1.1×10¹⁰ CFU/mL (e.g. when grown in fermenters). In another embodiment, the defined media supports growth to about 5×10⁹ CFU/mL (e.g. when grown in fermenters). In another embodiment, the defined media supports growth of viable bacteria (e.g. bacteria that can be cryopreserved without significant loss of viability) to about 3×10¹⁰ CFU/mL (e.g. when grown in fermenters). In another embodiment, the defined media supports growth to an OD₆₀₀ of about 2-10. In other embodiments, the defined media supports growth to another OD₆₀₀ value enumerated herein. In other embodiments, the defined media supports growth to another CFU/mL value enumerated herein. In another embodiment, the defined media supports growth to a density approximately equivalent to that obtained with TB. In another embodiment, the defined media supports growth to a density approximately equivalent to that obtained with LB.

In another embodiment, a defined media of methods and compositions of the present disclosure supports a growth rate of the Listeria strain of about 0.25 h⁻¹ (Examples). In another embodiment, the growth rate is about 0.15 h⁻¹. In another embodiment, the growth rate is about 0.2 h⁻¹. In another embodiment, the growth rate is about 0.3 h⁻¹. In another embodiment, the growth rate is about 0.4 h⁻¹. In another embodiment, the growth rate is about 0.5 h⁻¹. In another embodiment, the growth rate is about 0.6 h⁻¹. In another embodiment, the defined media supports a growth rate approximately equivalent to that obtained with TB. In another embodiment, the defined media supports a growth rate approximately equivalent to that obtained with LB.

In another embodiment, a peptide of the present disclosure is a fusion peptide. In another embodiment, “fusion peptide” refers to a peptide or polypeptide comprising 2 or more proteins linked together by peptide bonds or other chemical bonds. In another embodiment, the proteins are linked together directly by a peptide or other chemical bond. In another embodiment, the proteins are linked together with 1 or more AA (e.g. a “spacer”) between the 2 or more proteins.

In another embodiment, an immunotherapy of the present disclosure further comprises an adjuvant. The adjuvant utilized in methods and compositions of the present disclosure is, in another embodiment, a granulocyte/macrophage colony-stimulating factor (GM-CSF) protein. In another embodiment, the adjuvant comprises a GM-CSF protein. In another embodiment, the adjuvant is a nucleotide molecule encoding GM-CSF. In another embodiment, the adjuvant comprises a nucleotide molecule encoding GM-CSF. In another embodiment, the adjuvant is saponin QS21. In another embodiment, the adjuvant comprises saponin QS21. In another embodiment, the adjuvant is monophosphoryl lipid A. In another embodiment, the adjuvant comprises monophosphoryl lipid A. In another embodiment, the adjuvant is SBAS2. In another embodiment, the adjuvant comprises SBAS2. In another embodiment, the adjuvant is an unmethylated CpG-containing oligonucleotide. In another embodiment, the adjuvant comprises an unmethylated CpG-containing oligonucleotide. In another embodiment, the adjuvant is an immune-stimulating cytokine. In another embodiment, the adjuvant comprises an immune-stimulating cytokine. In another embodiment, the adjuvant is a nucleotide molecule encoding an immune-stimulating cytokine. In another embodiment, the adjuvant comprises a nucleotide molecule encoding an immune-stimulating cytokine. In another embodiment, the adjuvant is or comprises a quill glycoside. In another embodiment, the adjuvant is or comprises a bacterial mitogen. In another embodiment, the adjuvant is or comprises a bacterial toxin. In another embodiment, the adjuvant is or comprises any other adjuvant known in the art.

In another embodiment, a nucleotide of the present disclosure is operably linked to a promoter/regulatory sequence that drives expression of the encoded peptide in the Listeria strain. Promoter/regulatory sequences useful for driving constitutive expression of a gene are well known in the art and include, but are not limited to, for example, the P_{hlyA}, PActA, and p60 promoters of Listeria, the Streptococcus bac promoter, the Streptomyces griseus sgiA promoter, and the B. thuringiensis phaZ promoter. In another embodiment, inducible and tissue specific expression of the nucleic acid encoding a peptide of the present disclosure is accomplished by placing the nucleic acid encoding the peptide under the control of an inducible or tissue specific promoter/regulatory sequence. Examples of tissue specific or inducible promoter/regulatory sequences which are useful for his purpose include, but are not limited to the MMTV LTR inducible promoter, and the SV40 late enhancer/promoter. In another embodiment, a promoter that is induced in response to inducing agents such as metals, glucocorticoids, and the like, is utilized. Thus, it will be appreciated that the disclosure includes the use of any promoter/regulatory sequence, which is either known or unknown, and which is capable of driving expression of the desired protein operably linked thereto.

In another embodiment, the present disclosure provides a method of vaccinating a human subject against an antigen of interest, the method comprising the step of administering intravenously to the human subject a recombinant Listeria strain comprising or expressing the antigen of interest, wherein the first peptide is selected from (a) an N-terminal fragment of an LLO protein; (b) an ActA protein or N-terminal fragment thereof; and (c) a PEST-like sequence-containing peptide, thereby vaccinating a human subject against an antigen of interest.

In another embodiment, the present disclosure provides a method of vaccinating a human subject against an antigen of interest, the method comprising the step of administering intravenously to the human subject an immunogenic composition, comprising a fusion of a first peptide to the antigen of interest, wherein the first peptide is selected from (a) an N-terminal fragment of an LLO protein; (b) an ActA protein or N-terminal fragment thereof; and (c) a PEST-like sequence-containing peptide, thereby vaccinating a human subject against an antigen of interest.

In another embodiment, the present disclosure provides a method of vaccinating a human subject against an antigen of interest, the method comprising the step of administering intravenously to the human subject a recombinant Listeria strain comprising a recombinant polypeptide, the recombinant polypeptide comprising a first peptide fused to the antigen of interest, wherein the first peptide is selected from (a) an N-terminal fragment of an LLO protein; (b) an ActA protein or N-terminal fragment thereof; and (c) a PEST-like sequence-containing peptide, thereby vaccinating a human subject against an antigen of interest.

In another embodiment, the present disclosure provides a method of inducing a CTL response in a human subject against an antigen of interest, the method comprising the step of administering to the human subject a recombinant Listeria strain comprising or expressing the antigen of interest, thereby inducing a CTL response in a human subject against an antigen of interest. In another embodiment, the step of administering is intravenous administration.

In one embodiment, an antigen disclosed herein is a prostate specific antigen (PSA) or a chimeric HER2 antigen (cHER2).

The immune response induced by methods and compositions of the present disclosure is, in another embodiment, a T cell response. In another embodiment, the immune response comprises a T cell response. In another embodiment, the response is a CD8⁺ T cell response. In another embodiment, the response comprises a CD8⁺ T cell response.

The N-terminal LLO protein fragment of methods and compositions of the present disclosure comprises, in one embodiment, a sequence selected from SEQ ID Nos: 1-3. In another embodiment, the fragment comprises an LLO signal peptide. In another embodiment, the fragment consists of a sequence selected from SEQ ID Nos: 1-3. In another embodiment, the fragment consists essentially of a sequence selected from SEQ ID Nos: 1-3. In another embodiment, the fragment corresponds to a sequence selected from SEQ ID Nos: 1-3. In another embodiment, the fragment is homologous to a sequence selected from SEQ ID Nos: 1-3. In another embodiment, the fragment is homologous to a fragment of a sequence selected from SEQ ID Nos: 1-3. The ALLO used in some of the Examples was 416 AA long (exclusive of the signal sequence), as 88 residues from the amino terminus which is inclusive of the activation domain containing cysteine 484 were truncated. It will be clear to those skilled in the art that any ALLO without the activation domain, and in particular without cysteine 484, are suitable for methods and compositions of the present disclosure. In another embodiment, fusion of an E7 or E6 antigen to any ALLO, including a PEST AA sequence disclosed herein, enhances cell mediated and anti-tumor immunity of the antigen.

The LLO protein utilized to construct an immunotherapy disclosed herein comprises, in another embodiment, the sequence: MKKIMLVFITLILVSLPIAQQTEAKDASAFNKENSISSMAPPASPPASPKTPIEKKHA DEIDKYIQGLDYNKNNVLVYHGDAVTNVPPRKGYKDGNEYIVVEKKKKSINQNN ADIQVVNAISSLTYPGALVKANSELVENQPDVLPVKRDSLTLSIDLPGMTNQDNKI VVKNATKSNVNNAVNTLVERWNEKYAQAYPNVSAKIDYDDEMAYSESQLIAKF GTAFKAVNNSLNVNFGAISEGKMQEEVISFKQIYYNVNVNEPTRPSRFFGKAVTK EQLQALGVNAENPPAYISSVAYGRQVYLKLSTNSHSTKVKAAFDAAVSGKSVSG DVELTNIIKNSSFKAVIYGGSAKDEVQIIDGNLGDLRDILKKGATFNRETPGVPIAY TTNFLKDNELAVIKNNSEYIETTSKAYTDGKINIDHSGGYVAQFNISWDEVNYDPE GNEIVQHKNWSENNKSKLAHFTSSIYLPGNARNINVYAKECTGLAWEWWRTVID DRNLPLVKNRNISIWGTTLYPKYSNKVDNPIE (GenBank Accession No. P13128; SEQ ID NO: 1; nucleic acid sequence is set forth in GenBank Accession No. X15127). The first 25 AA of the proprotein corresponding to this sequence are the signal sequence and are cleaved from LLO when it is secreted by the bacterium. Thus, in this embodiment, the full length active LLO protein is 504 residues long. In another embodiment, the above LLO fragment is used as the source of the LLO fragment incorporated in a immunotherapy of the present disclosure.

In another embodiment, the N-terminal fragment of an LLO protein utilized in compositions and methods of the present disclosure has the sequence:

(SEQ ID NO: 2)
MKKIMLVFITLILVSLPIAQQTEAKDASAFNKENSISSVAPPASPPASPK
TPIEKKHADEIDKYIQGLDYNKNNVLVYHGDAVTNVPPRKGYKDGNEYIV
VEKKKKSINQNNADIQVVNAISSLTYPGALVKANSELVENQPDVLPVKRD
SLTLSIDLPGMTNQDNKIVVKNATKSNVNNAVNTLVERWNEKYAQAYSNV
SAKIDYDDEMAYSESQLIAKFGTAFKAVNNSLNVNFGAISEGKMQEEVIS
FKQIYYNVNVNEPTRPSRFFGKAVTKEQLQALGVNAENPPAYISSVAYGR
QVYLKLSTNSHSTKVKAAFDAAVSGKSVSGDVELTNIIKNSSFKAVIYGG
SAKDEVQIIDGNLGDLRDILKKGATFNRETPGVPIAYTTNFLKDNELAVI
KNNSEYIETTSKAYTDGKINIDHSGGYVAQFNISWDEVNYD.

In another embodiment, the LLO fragment corresponds to about AA 20-442 of an LLO protein utilized herein.

In another embodiment, the LLO fragment has the sequence:

(SEQ ID NO: 3)
MKKIMLVFITLILVSLPIAQQTEAKDASAFNKENSISSVAPPASPPASPK
TPIEKKHADEIDKYIQGLDYNKNNVLVYHGDAVTNVPPRKGYKDGNEYIV
VEKKKKSINQNNADIQVVNAISSLTYPGALVKANSELVENQPDVLPVKRD
SLTLSIDLPGMTNQDNKIVVKNATKSNVNNAVNTLVERWNEKYAQAYSNV
SAKIDYDDEMAYSESQLIAKFGTAFKAVNNSLNVNFGAISEGKMQEEVIS
FKQIYYNVNVNEPTRPSRFFGKAVTKEQLQALGVNAENPPAYISSVAYGR
QVYLKLSTNSHSTKVKAAFDAAVSGKSVSGDVELTNIIKNSSFKAVIYGG
SAKDEVQIIDGNLGDLRDILKKGATFNRETPGVPIAYTTNFLKDNELAVI
KNNSEYIETTSKAYTD.

In another embodiment, “N-terminal LLO,” “truncated LLO,” or “ALLO” are used interchangeably herein and refer to a fragment of a listeriolysin O (LLO) protein that comprises a putative PEST domain. In another embodiment, the terms refer to an LLO fragment that comprises a PEST sequence. In another embodiment, the terms refer to an LLO fragment that does not contain the activation domain at the amino terminus and does not include cysteine 484. In another embodiment, the terms refer to a sequence comprising a sequence selected from SEQ ID Nos 1-3. In another embodiment, the terms refer to an LLO that lack the cholesterol binding domain (CBD). In another embodiment, the terms refer to an LLO fragment that is not hemolytic. In another embodiment, the LLO fragment is rendered non-hemolytic by deletion or mutation of the activation domain. In another embodiment, the LLO fragment is rendered non-hemolytic by deletion or mutation of cysteine 484. In another embodiment, the LLO fragment is rendered non-hemolytic by deletion or mutation at another location.

In another embodiment, the LLO fragment consists of about the first 441 AA of the LLO protein. In another embodiment, the LLO fragment consists of about the first 420 AA of LLO. In another embodiment, the LLO fragment is a non-hemolytic form of the LLO protein.

In another embodiment, the LLO fragment contains residues of a homologous LLO protein that correspond to one of the above AA ranges. The residue numbers need not, in another embodiment, correspond exactly with the residue numbers enumerated above; e.g. if the homologous LLO protein has an insertion or deletion, relative to an LLO protein utilized herein, then the residue numbers can be adjusted accordingly.

In another embodiment, the LLO fragment is any other LLO fragment known in the art.

In another embodiment, the recombinant Listeria strain is administered to the human subject at a dose of 1×10⁹-3.31×10¹⁰ CFU. In another embodiment, the dose is 5-500×10⁸ CFU. In another embodiment, the dose is 7-500×10⁸ CFU. In another embodiment, the dose is 10-500×10⁸ CFU. In another embodiment, the dose is 20-500×10⁸ CFU. In another embodiment, the dose is 30-500×10⁸ CFU. In another embodiment, the dose is 50-500×10⁸ CFU. In another embodiment, the dose is 70-500×10⁸ CFU. In another embodiment, the dose is 100-500×10⁸ CFU. In another embodiment, the dose is 150-500×10⁸ CFU. In another embodiment, the dose is 5-300×10⁸ CFU. In another embodiment, the dose is 5-200×10⁸ CFU. In another embodiment, the dose is 5-150×10⁸ CFU. In another embodiment, the dose is 5-100×10⁸ CFU. In another embodiment, the dose is 5-70×10⁸ CFU. In another embodiment, the dose is 5-50×10⁸ CFU. In another embodiment, the dose is 5-30×10⁸ CFU. In another embodiment, the dose is 5-20×10⁸ CFU. In another embodiment, the dose is 1-30×10⁹ CFU. In another embodiment, the dose is 1-20×10⁹ CFU. In another embodiment, the dose is 2-30×10⁹ CFU. In another embodiment, the dose is 1-10×10⁹ CFU. In another embodiment, the dose is 2-10×10⁹ CFU. In another embodiment, the dose is 3-10×10⁹ CFU. In another embodiment, the dose is 2-7×10⁹ CFU. In another embodiment, the dose is 2-5×10⁹ CFU. In another embodiment, the dose is 3-5×10⁹ CFU.

In another embodiment, the dose is 1×10⁹ organisms. In another embodiment, the dose is 1.5×10⁹ organisms. In another embodiment, the dose is 2×10⁹ organisms. In another embodiment, the dose is 3×10⁹ organisms. In another embodiment, the dose is 4×10⁹ organisms. In another embodiment, the dose is 5×10⁹ organisms. In another embodiment, the dose is 6×10⁹ organisms. In another embodiment, the dose is 7×10⁹ organisms. In another embodiment, the dose is 8×10⁹ organisms. In another embodiment, the dose is 10×10⁹ organisms. In another embodiment, the dose is 1.5×10¹⁰ organisms. In another embodiment, the dose is 2×10¹⁰ organisms. In another embodiment, the dose is 2.5×10¹⁰ organisms. In another embodiment, the dose is 3×10¹⁰ organisms. In another embodiment, the dose is 3.3×10¹⁰ organisms. In another embodiment, the dose is 4×10¹⁰ organisms. In another embodiment, the dose is 5×10¹⁰ organisms.

In another embodiment, the recombinant polypeptide of methods of the present disclosure is expressed by the recombinant Listeria strain. In another embodiment, the expression is mediated by a nucleotide molecule carried by the recombinant Listeria strain.

In another embodiment, the recombinant Listeria strain expresses the recombinant polypeptide by means of a plasmid that encodes the recombinant polypeptide. In another embodiment, the plasmid comprises a gene encoding a bacterial transcription factor. In another embodiment, the plasmid encodes a Listeria transcription factor. In another embodiment, the transcription factor is prfA. In another embodiment, the transcription factor is any other transcription factor known in the art. In another embodiment, the recombinant Listeria is an attenuated auxotrophic strain. In another embodiment, the recombinant Listeria is an Lm-LLO-E7 strain described in U.S. Pat. No. 8,114,414, which is incorporated by reference herein in its entirety.

In one embodiment the attenuated strain is Lm dal(−)dat(−) (Lmdd). In another embodiment, the attenuated strains is Lm dal(−)dat(−)ΔactA (LmddA). LmddA is based on a Listeria immunotherapy vector which is attenuated due to the deletion of virulence gene actA and retains the plasmid for a desired heterologous antigen or truncated LLO expression in vivo and in vitro by complementation of dal gene.

In another embodiment, the Listeria strain is an auxotrophic mutant. In another embodiment, the Listeria strain is deficient in a gene encoding a vitamin synthesis gene. In another embodiment, the Listeria strain is deficient in a gene encoding pantothenic acid synthase.

In one embodiment, the generation of AA strains of Listeria deficient in D-alanine, for example, may be accomplished in a number of ways that are well known to those of skill in the art, including deletion mutagenesis, insertion mutagenesis, and mutagenesis which results in the generation of frameshift mutations, mutations which cause premature termination of a protein, or mutation of regulatory sequences which affect gene expression. In another embodiment, mutagenesis can be accomplished using recombinant DNA techniques or using traditional mutagenesis technology using mutagenic chemicals or radiation and subsequent selection of mutants. In another embodiment, deletion mutants are preferred because of the accompanying low probability of reversion of the auxotrophic phenotype. In another embodiment, mutants of D-alanine which are generated according to the protocols presented herein may be tested for the ability to grow in the absence of D-alanine in a simple laboratory culture assay. In another embodiment, those mutants which are unable to grow in the absence of this compound are selected for further study.

In another embodiment, in addition to the aforementioned D-alanine associated genes, other genes involved in synthesis of a metabolic enzyme, as provided herein, may be used as targets for mutagenesis of Listeria.

In one embodiment, a plasmid disclosed herein comprises an open reading frame encoding a metabolic enzyme that complements an endogenous gene mutation. In another embodiment, the metabolic enzyme complements an endogenous metabolic gene that is lacking in the remainder of the chromosome of the recombinant bacterial strain. In one embodiment, the endogenous metabolic gene is mutated in the chromosome. In another embodiment, the endogenous metabolic gene is deleted from the chromosome. In another embodiment, the metabolic enzyme is an amino acid metabolism enzyme. In another embodiment, the metabolic enzyme catalyzes a formation of an amino acid used for a cell wall synthesis in the recombinant Listeria strain. In another embodiment, the metabolic enzyme is an alanine racemase enzyme. In another embodiment, the metabolic enzyme is a D-amino acid transferase enzyme. In another embodiment, the metabolic enzyme is a D-alanine racemase enzyme.

In one embodiment, the auxotrophic Listeria strain comprises an episomal expression vector comprising a metabolic enzyme that complements the auxotrophy of the auxotrophic Listeria strain. In another embodiment, the construct is contained in the Listeria strain in an episomal fashion. In another embodiment, the foreign antigen is expressed from a vector harbored by the recombinant Listeria strain. In another embodiment, the episomal expression vector lacks an antibiotic resistance marker. In one embodiment, an antigen of the methods and compositions disclosed herein is fused to an polypeptide comprising a LLO sequence.

In another embodiment the attenuated strain is LmddA. In another embodiment, the attenuated strain is LmΔactA. In another embodiment, the attenuated strain is LmAPrfA. In another embodiment, the attenuated strain is LmΔPlcB. In another embodiment, the attenuated strain is LmΔPlcA. In another embodiment, the strain is the double mutant or triple mutant of any of the above-mentioned strains. In another embodiment, this strain exerts a strong adjuvant effect which is an inherent property of a Listeria-based immunotherapy. In another embodiment, this strain is constructed from the EGD Listeria backbone. In another embodiment, the strain used in the disclosure is a Listeria strain that expresses a non-hemolytic LLO.

In another embodiment, the Listeria strain is deficient in an amino acid (AA) metabolism enzyme. In another embodiment, the Listeria strain is deficient in a D-glutamic acid synthase gene. In another embodiment, the Listeria strain is deficient in the dat gene. In another embodiment, the Listeria strain is deficient in the dal gene. In another embodiment, the Listeria strain is deficient in the dga gene. In another embodiment, the Listeria strain is deficient in a gene involved in the synthesis of diaminopimelic acid. CysK. In another embodiment, the gene is vitamin-B12 independent methionine synthase. In another embodiment, the gene is trpA. In another embodiment, the gene is trpB. In another embodiment, the gene is trpE. In another embodiment, the gene is asnB. In another embodiment, the gene is gltD. In another embodiment, the gene is gltB. In another embodiment, the gene is leuA. In another embodiment, the gene is argG. In another embodiment, the gene is thrC. In another embodiment, the Listeria strain is deficient in one or more of the genes described hereinabove.

In another embodiment, the Listeria strain is deficient in a synthase gene. In another embodiment, the gene is an AA synthesis gene. In another embodiment, the gene is folP. In another embodiment, the gene is dihydrouridine synthase family protein. In another embodiment, the gene is ispD. In another embodiment, the gene is ispF. In another embodiment, the gene is phosphoenolpyruvate synthase. In another embodiment, the gene is hisF. In another embodiment, the gene is hisH. In another embodiment, the gene is fliI. In another embodiment, the gene is ribosomal large subunit pseudouridine synthase. In another embodiment, the gene is ispD. In another embodiment, the gene is bifunctional GMP synthase/glutamine amidotransferase protein. In another embodiment, the gene is cobS. In another embodiment, the gene is cobB. In another embodiment, the gene is cbiD. In another embodiment, the gene is uroporphyrin-III C-methyltransferase/uroporphyrinogen-III synthase. In another embodiment, the gene is cobQ. In another embodiment, the gene is uppS. In another embodiment, the gene is truB. In another embodiment, the gene is dxs. In another embodiment, the gene is mvaS. In another embodiment, the gene is dapA. In another embodiment, the gene is ispG. In another embodiment, the gene is folC. In another embodiment, the gene is citrate synthase. In another embodiment, the gene is argJ. In another embodiment, the gene is 3-deoxy-7-phosphoheptulonate synthase. In another embodiment, the gene is indole-3-glycerol-phosphate synthase. In another embodiment, the gene is anthranilate synthase/glutamine amidotransferase component. In another embodiment, the gene is menB. In another embodiment, the gene is menaquinone-specific isochorismate synthase. In another embodiment, the gene is phosphoribosylformylglycinamidine synthase I or II. In another embodiment, the gene is phosphoribosylaminoimidazole-succinocarboxamide synthase. In another embodiment, the gene is carB. In another embodiment, the gene is carA. In another embodiment, the gene is thyA. In another embodiment, the gene is mgsA. In another embodiment, the gene is aroB. In another embodiment, the gene is hepB. In another embodiment, the gene is rluB. In another embodiment, the gene is ilvB. In another embodiment, the gene is ilvN. In another embodiment, the gene is alsS. In another embodiment, the gene is fabF. In another embodiment, the gene is fabH. In another embodiment, the gene is pseudouridine synthase. In another embodiment, the gene is pyrG. In another embodiment, the gene is truA. In another embodiment, the gene is pabB. In another embodiment, the gene is an atp synthase gene (e.g. atpC, atpD-2, aptG, atpA-2, etc).

In another embodiment, the gene is phoP. In another embodiment, the gene is aroA. In another embodiment, the gene is aroC. In another embodiment, the gene is aroD. In another embodiment, the gene is plcB.

In one embodiment, provided herein is a nucleic acid molecule that is used to transform the Listeria in order to arrive at a recombinant Listeria. In another embodiment, the nucleic acid provided herein used to transform Listeria lacks a virulence gene. In another embodiment, the nucleic acid molecule is integrated into the Listeria genome and carries a non-functional virulence gene. In another embodiment, the virulence gene is mutated in the recombinant Listeria. In yet another embodiment, the nucleic acid molecule is used to inactivate the endogenous gene present in the Listeria genome. In yet another embodiment, the virulence gene is an actA gene, an inlA gene, and inlB gene, an inlC gene, inlJ gene, a plbC gene, a bsh gene, or a prfA gene. It is to be understood by a skilled artisan, that the virulence gene can be any gene known in the art to be associated with virulence in the recombinant Listeria.

In yet another embodiment the Listeria strain is an inlA mutant, an inlB mutant, an inlC mutant, an inlJ mutant, prfA mutant, actA mutant, a dal/dat mutant, a prfA mutant, a plcB deletion mutant, or a double mutant lacking both plcA and plcB or actA and inlB. In another embodiment, the Listeria comprise a mutation, deletion or inactivation of these genes individually or in combination. In another embodiment, the Listeria provided herein lack each one of genes. In another embodiment, the Listeria provided herein lack at least one and up to ten of any gene disclosed herein, including the actA, and dal/dat genes. In another embodiment, the plasmid comprises a gene encoding a metabolic enzyme. In another embodiment, the metabolic enzyme is a bacterial metabolic enzyme. In another embodiment, the metabolic enzyme is a Listerial metabolic enzyme. In another embodiment, the metabolic enzyme is an amino acid metabolism enzyme. In another embodiment, the amino acid metabolism gene is involved in a cell wall synthesis pathway. In another embodiment, the metabolic enzyme is the product of a D-amino acid aminotransferase gene (dat). In another embodiment, the metabolic enzyme is the product of an alanine racemase gene (dal). In another embodiment, the metabolic enzyme is any other metabolic enzyme known in the art. In one embodiment, the metabolic gene, the virulence gene, etc. is lacking in a chromosome of the Listeria strain. In another embodiment, the metabolic gene, virulence gene, etc. is lacking in the chromosome and in any episomal genetic element of the Listeria strain. In another embodiment, the metabolic gene, virulence gene, etc. is lacking in the genome of the virulence strain. In one embodiment, the virulence gene is mutated in the chromosome. In another embodiment, the virulence gene is deleted from the chromosome. Each possibility represents a separate embodiment of the present disclosure.

In one embodiment, the recombinant Listeria strain provided herein is attenuated. In another embodiment, the recombinant Listeria lacks the actA virulence gene. In another embodiment, the recombinant Listeria lacks the prfA virulence gene. In another embodiment, the recombinant Listeria lacks the inlB gene. In another embodiment, the recombinant Listeria lacks both, the actA and inlB genes. In another embodiment, the recombinant Listeria strain provided herein comprise an inactivating mutation of the endogenous actA gene. In another embodiment, the recombinant Listeria strain disclosed herein comprise an inactivating mutation of the endogenous inlB gene. In another embodiment, the recombinant Listeria strain disclosed herein comprise an inactivating mutation of the endogenous inlC gene. In another embodiment, the recombinant Listeria strain provided herein comprise an inactivating mutation of the endogenous actA and inlB genes. In another embodiment, the recombinant Listeria strain disclosed herein comprise an inactivating mutation of the endogenous actA and inlC genes. In another embodiment, the recombinant Listeria strain provided herein comprise an inactivating mutation of the endogenous actA, inlB, and inlC genes. In another embodiment, the recombinant Listeria strain disclose herein comprise an inactivating mutation of the endogenous actA, inlB, and inlC genes. In another embodiment, the recombinant Listeria strain provided herein comprise an inactivating mutation of the endogenous actA, inlB, and inlC genes. In another embodiment, the recombinant Listeria strain disclosed herein comprise an inactivating mutation in any single gene or combination of the following genes: actA, dal, dat, inlB, inlC, prfA, plcA, plcB.

It will be appreciated by the skilled artisan that the term “mutation” and grammatical equivalents thereof, include any type of mutation or modification to the sequence (nucleic acid or amino acid sequence), and includes a deletion mutation, a truncation, an inactivation, a disruption, or a translocation. These types of mutations are readily known in the art.

In one embodiment, in order to select for an auxotrophic bacteria comprising a plasmid encoding a metabolic enzyme or a complementing gene provided herein, transformed auxotrophic bacteria are grown on a media that will select for expression of the amino acid metabolism gene or the complementing gene. In another embodiment, a bacteria auxotrophic for D-glutamic acid synthesis is transformed with a plasmid comprising a gene for D-glutamic acid synthesis, and the auxotrophic bacteria will grow in the absence of D-glutamic acid, whereas auxotrophic bacteria that have not been transformed with the plasmid, or are not expressing the plasmid encoding a protein for D-glutamic acid synthesis, will not grow. In another embodiment, a bacterium auxotrophic for D-alanine synthesis will grow in the absence of D-alanine when transformed and expressing the plasmid of the present disclosure if the plasmid comprises an isolated nucleic acid encoding an amino acid metabolism enzyme for D-alanine synthesis. Such methods for making appropriate media comprising or lacking necessary growth factors, supplements, amino acids, vitamins, antibiotics, and the like are well known in the art, and are available commercially (Becton-Dickinson, Franklin Lakes, N.J.).

In another embodiment, once the auxotrophic bacteria comprising the plasmid of the present disclosure have been selected on appropriate media, the bacteria are propagated in the presence of a selective pressure. Such propagation comprises growing the bacteria in media without the auxotrophic factor. The presence of the plasmid expressing an amino acid metabolism enzyme in the auxotrophic bacteria ensures that the plasmid will replicate along with the bacteria, thus continually selecting for bacteria harboring the plasmid. The skilled artisan, when equipped with the present disclosure and methods herein will be readily able to scale-up the production of the Listeria immunotherapy vector by adjusting the volume of the media in which the auxotrophic bacteria comprising the plasmid are growing.

The skilled artisan will appreciate that, in another embodiment, other auxotroph strains and complementation systems are adopted for the use with this disclosure.

In one embodiment, the N-terminal LLO protein fragment and heterologous antigen are fused directly to one another. In another embodiment, the genes encoding the N-terminal LLO protein fragment and heterologous antigen are fused directly to one another. In another embodiment, the N-terminal LLO protein fragment and heterologous antigen are operably attached via a linker peptide. In another embodiment, the N-terminal LLO protein fragment and heterologous antigen are attached via a heterologous peptide. In another embodiment, the N-terminal LLO protein fragment is N-terminal to the heterologous antigen. In another embodiment, the N-terminal LLO protein fragment is expressed and used alone, i.e., in unfused form. In another embodiment, an N-terminal LLO protein fragment is the N-terminal-most portion of the fusion protein. In another embodiment, a truncated LLO is truncated at the C-terminal to arrive at an N-terminal LLO. In another embodiment, a truncated LLO is a non-hemolytic LLO.

In one embodiment, the recombinant Listeria strain of the compositions and methods as provided herein comprise a first or second nucleic acid molecule that encodes a Prostate Specific Antigen (PSA), which in one embodiment, is a marker for prostate cancer that is highly expressed by prostate tumors. In one embodiment, PSA is a kallikrein serine protease (KLK3) secreted by prostatic epithelial cells, which in one embodiment, is widely used as a marker for prostate cancer. As used herein, the terms PSA and KLK3 are interchangeable having all the same meanings and qualities.

In one embodiment, the recombinant Listeria strain as provided herein comprises a nucleic acid molecule encoding a tumor associated antigen. In one embodiment, a tumor associated antigen comprises a KLK3 polypeptide or a fragment thereof. In one embodiment, the recombinant Listeria strain as provided herein comprises a nucleic acid molecule encoding KLK3 protein.

In another embodiment, the KLK3 protein comprises the sequence: MWVPVVFLTLSVTWIGAAPLILSRIVGGWECEKHSQPWQVLVASRGRAVCGGVL VHPQWVLTAAHCIRNKSVILLGRHSLFHPEDTGQVFQVSHSFPHPLYDMSLLKNR FLRPGDDSSHDLMLLRLSEPAELTDAVKVMDLPTQEPALGTTCYASGWGSIEPEE FLTPKKLQCVDLHVISNDVCAQVHPQKVTKFMLCAGRWTGGKSTCSGDSGGPLV CNGVLQGITSWGSEPCALPERPSLYTKVVHYRKWIKDTIVANP (SEQ ID NO: 4; GenBank Accession No. CAA32915). In another embodiment, the KLK3 protein is a homologue of SEQ ID NO: 4. In another embodiment, the KLK3 protein is a variant of SEQ ID NO: 4. In another embodiment, the KLK3 protein is an isomer of SEQ ID NO: 4.

In another embodiment, the KLK3 protein is a fragment of SEQ ID NO: 4.

In another embodiment, the KLK3 protein comprising the sequence: IVGGWECEKHSQPWQVLVASRGRAVCGGVLVHPQWVLTAAHCIRNKSVI LLGRHSLFHPEDTGQVFQVSHSFPHPLYDMSLLKNRFLRPGDDSSHDLMLLRLSEP AELTDAVKVMDLPTQEPALGTTCYASGWGSIEPEEFLTPKKLQCVDLHVISNDVC AQVHPQKVTKFMLCAGRWTGGKSTCSGDSGGPLVCYGVLQGITSWGSEPCALPE RPSLYTKVVHYRKWIKDTIVANP (SEQ ID NO: 5). In another embodiment, the KLK3 protein is a homologue of SEQ ID NO: 5. In another embodiment, the KLK3 protein is a variant of SEQ ID NO: 5. In another embodiment, the KLK3 protein is an isomer of SEQ ID NO: 5. In another embodiment, the KLK3 protein is a fragment of SEQ ID NO: 5.

In another embodiment, the KLK3 protein comprising the sequence:

IVGGWECEKHSQPWQVLVASRGRAVCGGVLVHPQWVLTAAHCIRNKSVI LLGRHSLFHPEDTGQVFQVSHSFPHPLYDMSLLKNRFLRPGDDSSHDLMLLRLSEP AELTDAVKVMDLPTQEPALGTTCYASGWGSIEPEEFLTPKKLQCVDLHVISNDVC AQVHPQKVTKFMLCAGRWTGGKSTCSGDSGGPLVCNGVLQGITSWGSEPCALPE RPSLYTKVVHYRKWIKDTIVANP (SEQ ID NO: 6; GenBank Accession No. AAA59995.1). In another embodiment, the KLK3 protein is a homologue of SEQ ID NO: 6. In another embodiment, the KLK3 protein is a variant of SEQ ID NO: 6. In another embodiment, the KLK3 protein is an isomer of SEQ ID NO: 6. In another embodiment, the KLK3 protein is a fragment of SEQ ID NO: 6.

In another embodiment, the KLK3 protein is encoded by a nucleotide molecule comprising the sequence:

ggtgtcttaggcacactggtcttggagtgcaaaggatctaggcacgtgaggctttgtatgaagaatcggggatcgtacc caccccctgtttctgtttcatcctgggcatgtctcctctgcctttgtcccctagatgaagtctccatgagctacaagggcctggtgcatc cagggtgatctagtaattgcagaacagcaagtgctagctctccctccccttccacagctctgggtgtgggagggggttgtccagcc tccagcagcatggggagggccttggtcagcctctgggtgccagcagggcaggggcggagtcctggggaatgaaggttttatag ggctcctgggggaggctccccagccccaagcttaccacctgcacccggagagctgtgtcaccatgtgggtcccggttgtcttcct caccctgtccgtgacgtggattggtgagaggggccatggttggggggatgcaggagagggagccagccctgactgtcaagctg aggctctttcccccccaacccagcaccccagcccagacagggagctgggctcttttctgtctctcccagccccacttcaagcccat acccccagtcccctccatattgcaacagtcctcactcccacaccaggtccccgctccctcccacttaccccagaactttcttcccatt tgcccagccagctccctgctcccagctgctttactaaaggggaagttcctgggcatctccgtgtttctctttgtggggctcaaaacct ccaaggacctctctcaatgccattggttccttggaccgtatcactggtccatctcctgagcccctcaatcctatcacagtctactgact tttcccattcagctgtgagtgtccaaccctatcccagagaccttgatgcttggcctcccaatcttgccctaggatacccagatgccaa ccagacacctccttctttcctagccaggctatctggcctgagacaacaaatgggtccctcagtctggcaatgggactctgagaactc ctcattccctgactcttagccccagactcttcattcagtggcccacattttccttaggaaaaacatgagcatccccagccacaactgc cagctctctgagtccccaaatctgcatccttttcaaaacctaaaaacaaaaagaaaaacaaataaaacaaaaccaactcagaccag aactgttttctcaacctgggacttcctaaactttccaaaaccttcctcttccagcaactgaacctcgccataaggcacttatccctggtt cctagcaccccttatcccctcagaatccacaacttgtaccaagtttcccttctcccagtccaagaccccaaatcaccacaaaggacc caatccccagactcaagatatggtctgggcgctgtcttgtgtctcctaccctgatccctgggttcaactctgctcccagagcatgaag cctctccaccagcaccagccaccaacctgcaaacctagggaagattgacagaattcccagcctttcccagctccccctgcccatg tcccaggactcccagccttggttctctgcccccgtgtcttttcaaacccacatcctaaatccatctcctatccgagtcccccagttccc cctgtcaaccctgattcccctgatctagcaccccctctgcaggcgctgcgcccctcatcctgtctcggattgtgggaggctgggag tgcgagaagcattcccaaccctggcaggtgcttgtggcctctcgtggcagggcagtctgcggcggtgttctggtgcacccccagt gggtcctcacagctgcccactgcatcaggaagtgagtaggggcctggggtctggggagcaggtgtctgtgtcccagaggaata acagctgggcattttccccaggataacctctaaggccagccttgggactgggggagagagggaaagttctggttcaggtcacatg gggaggcagggttggggctggaccaccctccccatggctgcctgggtctccatctgtgtccctctatgtctctttgtgtcgctttcatt atgtctcttggtaactggcttcggttgtgtctctccgtgtgactattttgttctctctctccctctcttctctgtcttcagtctccatatctccc cctctctctgtccttctctggtccctctctagccagtgtgtctcaccctgtatctctctgccaggctctgtctctcggtctctgtctcacct gtgccttctccctactgaacacacgcacgggatgggcctgggggaccctgagaaaaggaagggctttggctgggcgcggtggc tcacacctgtaatcccagcactttgggaggccaaggcaggtagatcacctgaggtcaggagttcgagaccagcctggccaactg gtgaaaccccatctctactaaaaatacaaaaaattagccaggcgtggtggcgcatgcctgtagtcccagctactcaggagctgag ggaggagaattgcattgaacctggaggttgaggttgcagtgagccgagaccgtgccactgcactccagcctgggtgacagagtg agactccgcctcaaaaaaaaaaaaaaaaaaaaaaaaaaaaaagaaaagaaaagaaaagaaaaggaagtgttttatccctgatgt gtgtgggtatgagggtatgagagggcccctctcactccattccttctccaggacatccctccactcttgggagacacagagaagg gctggttccagctggagctgggaggggcaattgagggaggaggaaggagaagggggaaggaaaacagggtatgggggaaa ggaccctggggagcgaagtggaggatacaaccttgggcctgcaggcaggctacctacccacttggaaacccacgccaaagcc gcatctacagctgagccactctgaggcctcccctccccggcggtccccactcagctccaaagtctctctcccttttctctcccacact ttatcatcccccggattcctctctacttggttctcattcttcctttgacttcctgcttccctttctcattcatctgtttctcactttctgcctggtt gttctcccctctgccctttcattctctctgcccttttaccctcttccttttcccttggttctctcagttctgtatctgcccttcaccctctcaca ctgctgtttcccaactcgttgtctgtattttggcctgaactgtgtcttcccaaccctgtgttttctcactgtttctttttctcttttggagcctcc tccttgctcctctgtcccttctctctttccttatcatcctcgctcctcattcctgcgtctgcttcctccccagcaaaagcgtgatcttgctgg gtcggcacagcctgtttcatcctgaagacacaggccaggtatttcaggtcagccacagcttcccacacccgctctacgatatgagc ctcctgaagaatcgattcctcaggccaggtgatgactccagccacgacctcatgctgctccgcctgtcagagcctgccgagctca cggatgctgtgaaggtcatggacctgcccacccaggagccagcactggggaccacctgctacgcctcaggctggggcagcatt gaaccagaggagtgtacgcctgggccagatggtgcagccgggagcccagatgcctgggtctgagggaggaggggacagga ctcctgggtctgagggaggagggccaaggaaccaggtggggtccagcccacaacagtgtttttgcctggcccgtagtcttgacc ccaaagaaacttcagtgtgtggacctccatgttatttccaatgacgtgtgtgcgcaagttcaccctcagaaggtgaccaagttcatgc tgtgtgctggacgctggacagggggcaaaagcacctgctcggtgagtcatccctactcccaagatcttgagggaaaggtgagtg ggaccttaattctgggctggggtctagaagccaacaaggcgtctgcctcccctgctccccagctgtagccatgccacctccccgt gtctcatctcattccctccttccctcttctttgactccctcaaggcaataggttattcttacagcacaactcatctgttcctgcgttcagca cacggttactaggcacctgctatgcacccagcactgccctagagcctgggacatagcagtgaacagacagagagcagcccctc ccttctgtagcccccaagccagtgaggggcacaggcaggaacagggaccacaacacagaaaagctggagggtgtcaggagg tgatcaggctctcggggagggagaaggggtggggagtgtgactgggaggagacatcctgcagaaggtgggagtgagcaaac acctgcgcaggggaggggagggcctgcggcacctgggggagcagagggaacagcatctggccaggcctgggaggagggg cctagagggcgtcaggagcagagaggaggttgcctggctggagtgaaggatcggggcagggtgcgagagggaacaaagga cccctcctgcagggcctcacctgggccacaggaggacactgcttttcctctgaggagtcaggaactgtggatggtgctggacag aagcaggacagggcctggctcaggtgtccagaggctgcgctggcctcctatgggatcagactgcagggagggagggcagca gggatgtggagggagtgatgatggggctgacctgggggtggctccaggcattgtccccacctgggcccttacccagcctccctc acaggctcctggccctcagtctctcccctccactccattctccacctacccacagtgggtcattctgatcaccgaactgaccatgcc agccctgccgatggtcctccatggctccctagtgccctggagaggaggtgtctagtcagagagtagtcctggaaggtggcctctg tgaggagccacggggacagcatcctgcagatggtcctggcccttgtcccaccgacctgtctacaaggactgtcctcgtggaccct cccctctgcacaggagctggaccctgaagtcccttcctaccggccaggactggagcccctacccctctgttggaatccctgccca ccttcttctggaagtcggctctggagacatttctctcttcttccaaagctgggaactgctatctgttatctgcctgtccaggtctgaaag ataggattgcccaggcagaaactgggactgacctatctcactctctccctgcttttacccttagggtgattctgggggcccacttgtct gtaatggtgtgcttcaaggtatcacgtcatggggcagtgaaccatgtgccctgcccgaaaggccttccctgtacaccaaggtggtg cattaccggaagtggatcaaggacaccatcgtggccaacccctgagcacccctatcaagtccctattgtagtaaacttggaacctt ggaaatgaccaggccaagactcaagcctcccagttctactgacctttgtccttaggtgtgaggtccagggttgctaggaaaagaa atcagcagacacaggtgtagaccagagtgtttcttaaatggtgtaattttgtcctctctgtgtcctggggaatactggccatgcctgga gacatatcactcaatttctctgaggacacagttaggatggggtgtctgtgttatttgtgggatacagagatgaaagaggggtgggat cc (SEQ ID NO: 7; GenBank Accession No. X14810). In another embodiment, the KLK3 protein is encoded by residues 401 . . . 446, 1688 . . . 1847, 3477 . . . 3763, 3907 . . . 4043, and 5413 . . . 5568 of SEQ ID NO: 7. In another embodiment, the KLK3 protein is encoded by a homologue of SEQ ID NO: 7. In another embodiment, the KLK3 protein is encoded by a variant of SEQ ID NO: 7. In another embodiment, the KLK3 protein is encoded by an isomer of SEQ ID NO: 7. In another embodiment, the KLK3 protein is encoded by a fragment of SEQ ID NO: 7.

In another embodiment, the KLK3 protein comprises the sequence: MWVPVVFLTLSVTWIGAAPLILSRIVGGWECEKHSQPWQVLVASRGRAVCGGVL VHPQWVLTAAHCIRNKSVILLGRHSLFHPEDTGQVFQVSHSFPHPLYDMSLLKNR FLRPGDDSSHDLMLLRLSEPAELTDAVKVMDLPTQEPALGTTCYASGWGSIEPEE FLTPKKLQCVDLHVISNDVCAQVHPQKVTKFMLCAGRWTGGKSTCSWVILITELT MPALPMVLHGSLVPWRGGV (SEQ ID NO: 8; GenBank Accession No. NP_001019218). In another embodiment, the KLK3 protein is a homologue of SEQ ID NO: 8. In another embodiment, the KLK3 protein is a variant of SEQ ID NO: 8. In another embodiment, the KLK3 protein is an isomer of SEQ ID NO: 8. In another embodiment, the KLK3 protein is a fragment of SEQ ID NO: 8.

In another embodiment, the KLK3 protein is encoded by a nucleotide molecule having the sequence:

agccccaagcttaccacctgcacccggagagctgtgtcaccatgtgggtcccggttgtcttcctcaccctgtccgtgac gtggattggtgctgcacccctcatcctgtctcggattgtgggaggctgggagtgcgagaagcattcccaaccctggcaggtgcttg tggcctctcgtggcagggcagtctgcggcggtgttctggtgcacccccagtgggtcctcacagctgcccactgcatcaggaaca aaagcgtgatcttgctgggtcggcacagcctgtttcatcctgaagacacaggccaggtatttcaggtcagccacagcttcccacac ccgctctacgatatgagcctcctgaagaatcgattcctcaggccaggtgatgactccagccacgacctcatgctgctccgcctgtc agagcctgccgagctcacggatgctgtgaaggtcatggacctgcccacccaggagccagcactggggaccacctgctacgcct caggctggggcagcattgaaccagaggagttcttgaccccaaagaaacttcagtgtgtggacctccatgttatttccaatgacgtgt gtgcgcaagttcaccctcagaaggtgaccaagttcatgctgtgtgctggacgctggacagggggcaaaagcacctgctcgtggg tcattctgatcaccgaactgaccatgccagccctgccgatggtcctccatggctccctagtgccctggagaggaggtgtctagtca gagagtagtcctggaaggtggcctctgtgaggagccacggggacagcatcctgcagatggtcctggcccttgtcccaccgacct gtctacaaggactgtcctcgtggaccctcccctctgcacaggagctggaccctgaagtcccttccccaccggccaggactggag cccctacccctctgttggaatccctgcccaccttcttctggaagtcggctctggagacatttctctcttcttccaaagctgggaactgc tatctgttatctgcctgtccaggtctgaaagataggattgcccaggcagaaactgggactgacctatctcactctctccctgcttttac ccttagggtgattctgggggcccacttgtctgtaatggtgtgcttcaaggtatcacgtcatggggcagtgaaccatgtgccctgccc gaaaggccttccctgtacaccaaggtggtgcattaccggaagtggatcaaggacaccatcgtggccaacccctgagcaccccta tcaaccccctattgtagtaaacttggaaccttggaaatgaccaggccaagactcaagcctccccagttctactgacctttgtccttag gtgtgaggtccagggttgctaggaaaagaaatcagcagacacaggtgtagaccagagtgtttcttaaatggtgtaattttgtcctctc tgtgtcctggggaatactggccatgcctggagacatatcactcaatttctctgaggacacagataggatggggtgtctgtgttatttgt ggggtacagagatgaaagaggggtgggatccacactgagagagtggagagtgacatgtgctggacactgtccatgaagcactg agcagaagctggaggcacaacgcaccagacactcacagcaaggatggagctgaaaacataacccactctgtcctggaggcact gggaagcctagagaaggctgtgagccaaggagggagggtcttcctttggcatgggatggggatgaagtaaggagagggactg gaccccctggaagctgattcactatggggggaggtgtattgaagtcctccagacaaccctcagatttgatgatttcctagtagaact cacagaaataaagagctgttatactgtg (SEQ ID NO: 9; GenBank Accession No. NM_001030047). In another embodiment, the KLK3 protein is encoded by residues 42-758 of SEQ ID NO: 9. In another embodiment, the KLK3 protein is encoded by a homologue of SEQ ID NO: 9. In another embodiment, the KLK3 protein is encoded by a variant of SEQ ID NO: 9. In another embodiment, the KLK3 protein is encoded by an isomer of SEQ ID NO: 9. In another embodiment, the KLK3 protein is encoded by a fragment of SEQ ID NO: 9. In another embodiment, a KLK3 protein is encoded by a nucleotide molecule comprising the sequence:

attgtgggaggctgggagtgcgagaagcattcccaaccctggcaggtgcttgtggcctctcgtggcagggcagtctgcggcggt gttctggtgcacccccagtgggtcctcacagctgcccactgcatcaggaacaaaagcgtgatcttgctgggtcggcacagcctgt ttcatcctgaagacacaggccaggtatttcaggtcagccacagcttcccacacccgctctacgatatgagcctcctgaagaatcga ttcctcaggccaggtgatgactccagccacgacctcatgctgctccgcctgtcagagcctgccgagctcacggatgctgtgaagg tcatggacctgcccacccaggagccagcactggggaccacctgctacgcctcaggctggggcagcattgaaccagaggagtt cttgaccccaaagaaacttcagtgtgtggacctccatgttatttccaatgacgtgtgtgcgcaagttcaccctcagaaggtgaccaa gttcatgctgtgtgctggacgctggacagggggcaaaagcacctgctcgggtgattctgggggcccacttgtctgttatggtgtgc ttcaaggtatcacgtcatggggcagtgaaccatgtgccctgcccgaaaggccttccctgtacaccaaggtggtgcattaccggaa gtggatcaaggacaccatcgtggccaacccc (SEQ ID NO: 10). In another embodiment, the KLK3 protein is encoded by a homologue of SEQ ID NO: 10. In another embodiment, the KLK3 protein is encoded by a variant of SEQ ID NO: 10. In another embodiment, the KLK3 protein is encoded by an isomer of SEQ ID NO: 10. In another embodiment, the KLK3 protein is encoded by a fragment of SEQ ID NO: 10.

In another embodiment, the KLK3 protein is encoded by a sequence set forth in one of the following GenBank Accession Numbers: BC005307, AJ310938, AJ310937, AF335478, AF335477, M27274, and M26663. In another embodiment, the KLK3 protein is encoded by a sequence set forth in one of the above GenBank Accession Numbers. Each possibility represents a separate embodiment of the methods and compositions as provided herein.

In another embodiment, the KLK3 protein is encoded by a sequence set forth in one of the following GenBank Accession Numbers: NM_001030050, NM_001030049, NM_001030048, NM_001030047, NM_001648, AJ459782, AJ512346, or AJ459784. Each possibility represents a separate embodiment of the methods and compositions as provided herein. In one embodiment, the KLK3 protein is encoded by a variation of any of the sequences described herein wherein the sequence lacks MWVPVVFLTLSVTWIGAAPLILSR (SEQ ID NO: 11).

In another embodiment, the KLK3 protein has the sequence that comprises a sequence set forth in one of the following GenBank Accession Numbers: X13943, X13942, X13940, X13941, and X13944. Each possibility represents a separate embodiment of the methods and compositions as provided herein.

In another embodiment, the KLK3 protein is any other KLK3 protein known in the art. In another embodiment, the KLK3 peptide is any other KLK3 peptide known in the art. In another embodiment, the KLK3 peptide is a fragment of any other KLK3 peptide known in the art.

“KLK3 peptide” refers, in another embodiment, to a full-length KLK3 protein. In another embodiment, the term refers to a fragment of a KLK3 protein. In another embodiment, the term refers to a fragment of a KLK3 protein that is lacking the KLK3 signal peptide. In another embodiment, the term refers to a KLK3 protein that contains the entire KLK3 sequence except the KLK3 signal peptide. “KLK3 signal sequence” refers, in another embodiment, to any signal sequence found in nature on a KLK3 protein. In another embodiment, a KLK3 protein of methods and compositions as provided herein does not contain any signal sequence.

In another embodiment, the kallikrein-related peptidase 3 (KLK3 protein) that is the source of a KLK3 peptide for use in the methods and compositions disclosed herein is a PSA protein. In another embodiment, the KLK3 protein is a P-30 antigen protein. In another embodiment, the KLK3 protein is a gamma-seminoprotein protein. In another embodiment, the KLK3 protein is a kallikrein 3 protein. In another embodiment, the KLK3 protein is a semenogelase protein. In another embodiment, the KLK3 protein is a seminin protein. In another embodiment, the KLK3 protein is any other type of KLK3 protein that is known in the art. Each possibility represents a separate embodiment of the methods and compositions as provided herein.

In another embodiment, the KLK3 protein is a splice variant 1 KLK3 protein. In another embodiment, the KLK3 protein is a splice variant 2 KLK3 protein. In another embodiment, the KLK3 protein is a splice variant 3 KLK3 protein. In another embodiment, the KLK3 protein is a transcript variant 1 KLK3 protein. In another embodiment, the KLK3 protein is a transcript variant 2 KLK3 protein. In another embodiment, the KLK3 protein is a transcript variant 3 KLK3 protein. In another embodiment, the KLK3 protein is a transcript variant 4 KLK3 protein. In another embodiment, the KLK3 protein is a transcript variant 5 KLK3 protein. In another embodiment, the KLK3 protein is a transcript variant 6 KLK3 protein. In another embodiment, the KLK3 protein is a splice variant RP5 KLK3 protein. In another embodiment, the KLK3 protein is any other splice variant KLK3 protein known in the art. In another embodiment, the KLK3 protein is any other transcript variant KLK3 protein known in the art.

In another embodiment, the KLK3 protein is a mature KLK3 protein. In another embodiment, the KLK3 protein is a pro-KLK3 protein. In another embodiment, the leader sequence has been removed from a mature KLK3 protein of methods and compositions as provided herein.

In another embodiment, the KLK3 protein that is the source of a KLK3 peptide of methods and compositions as provided herein is a human KLK3 protein. In another embodiment, the KLK3 protein is a primate KLK3 protein. In another embodiment, the KLK3 protein is a KLK3 protein of any other species known in the art. In another embodiment, one of the above KLK3 proteins is referred to in the art as a “KLK3 protein.”

In one embodiment, a recombinant polypeptide disclosed herein comprising a truncated LLO fused to a PSA protein disclosed herein is encoded by a sequence comprising:

ATGAAAAAAATAATGCTAGTTTTTATTACACTTATATTAGTTAGTCTACC AATTGCGCAACAAACTGAAGCAAAGGATGCATCTGCATTCAATAAAGAAAATT CAATTTCATCCATGGCACCACCAGCATCTCCGCCTGCAAGTCCTAAGACGCCAA TCGAAAAGAAACACGCGGATGAAATCGATAAGTATATACAAGGATTGGATTAC AATAAAAACAATGTATTAGTATACCACGGAGATGCAGTGACAAATGTGCCGCC AAGAAAAGGTTACAAAGATGGAAATGAATATATTGTTGTGGAGAAAAAGAAGA AATCCATCAATCAAAATAATGCAGACATTCAAGTTGTGAATGCAATTTCGAGCC TAACCTATCCAGGTGCTCTCGTAAAAGCGAATTCGGAATTAGTAGAAAATCAAC CAGATGTTCTCCCTGTAAAACGTGATTCATTAACACTCAGCATTGATTTGCCAG GTATGACTAATCAAGACAATAAAATAGTTGTAAAAAATGCCACTAAATCAAAC GTTAACAACGCAGTAAATACATTAGTGGAAAGATGGAATGAAAAATATGCTCA AGCTTATCCAAATGTAAGTGCAAAAATTGATTATGATGACGAAATGGCTTACAG TGAATCACAATTAATTGCGAAATTTGGTACAGCATTTAAAGCTGTAAATAATAG CTTGAATGTAAACTTCGGCGCAATCAGTGAAGGGAAAATGCAAGAAGAAGTCA TTAGTTTTAAACAAATTTACTATAACGTGAATGTTAATGAACCTACAAGACCTT CCAGATTTTTCGGCAAAGCTGTTACTAAAGAGCAGTTGCAAGCGCTTGGAGTGA ATGCAGAAAATCCTCCTGCATATATCTCAAGTGTGGCGTATGGCCGTCAAGTTT ATTTGAAATTATCAACTAATTCCCATAGTACTAAAGTAAAAGCTGCTTTTGATG CTGCCGTAAGCGGAAAATCTGTCTCAGGTGATGTAGAACTAACAAATATCATCA AAAATTCTTCCTTCAAAGCCGTAATTTACGGAGGTTCCGCAAAAGATGAAGTTC AAATCATCGACGGCAACCTCGGAGACTTACGCGATATTTTGAAAAAAGGCGCT ACTTTTAATCGAGAAACACCAGGAGTTCCCATTGCTTATACAACAAACTTCCTA AAAGACAATGAATTAGCTGTTATTAAAAACAACTCAGAATATATTGAAACAAC TTCAAAAGCTTATACAGATGGAAAAATTAACATCGATCACTCTGGAGGATACGT TGCTCAATTCAACATTTCTTGGGATGAAGTAAATTATGATCTCGAGattgtgggaggct gggagtgcgagaagcattcccaaccctggcaggtgcttgtggcctctcgtggcagggcagtctgcggcggtgttctggtgcaccc ccagtgggtcctcacagctgcccactgcatcaggaacaaagcgtgatcttgctgggtcggcacagcctgtttcatcctgaagac acaggccaggtatttcaggtcagccacagcttcccacacccgctctacgatatgagcctcctgaagaatcgattcctcaggccag gtgatgactccagccacgacctcatgctgctccgcctgtcagagcctgccgagctcacggatgctgtgaaggtcatggacctgcc cacccaggagccagcactggggaccacctgctacgcctcaggctggggcagcattgaaccagaggagttcttgaccccaaag aaacttcagtgtgtggacctccatgttatttccaatgacgtgtgtgcgcaagttcaccctcagaaggtgaccaagttcatgctgtgtg ctggacgctggacagggggcaaaagcacctgctcgggtgattctgggggcccacttgtctgttatggtgtgcttcaaggtatcacg tcatggggcagtgaaccatgtgccctgcccgaaaggccttccctgtacaccaaggtggtgcattaccggaagtggatcaaggac accatcgtggccaacccc (SEQ ID NO: 12). In another embodiment, the fusion protein is encoded by a homologue of SEQ ID No: 12. In another embodiment, the fusion protein is encoded by a variant of SEQ ID No: 12. In another embodiment, the fusion protein is encoded by an isomer of SEQ ID No: 12. In one embodiment, the “ctcgag” sequence within the fusion protein represents a Xho I restriction site used to ligate the tumor antigen to truncated LLO in the plasmid.

In another embodiment, a recombinant polypeptide disclosed herein comprising a truncated LLO fused to a PSA protein disclosed herein comprises the following sequence:

MKKIMLVFITLILVSLPIAQQTEAKDASAFNKENSISSMAPPASPPASPKTPIE KKHADEIDKYIQGLDYNKNNVLVYHGDAVTNVPPRKGYKDGNEYIVVEKKKKSIN QNNADIQVVNAISSLTYPGALVKANSELVENQPDVLPVKRDSLTLSIDLPGMTNQD NKIVVKNATKSNVNNAVNTLVERWNEKYAQAYPNVSAKIDYDDEMAYSESQLIA KFGTAFKAVNNSLNVNFGAISEGKMQEEVISFKQIYYNVNVNEPTRPSRFFGKAVT KEQLQALGVNAENPPAYIS SVAYGRQVYLKLSTNSHSTKVKAAFDAAVSGKSVSG DVELTNIIKNS SFKAVIYGGSAKDEVQIIDGNLGDLRDILKKGATFNRETPGVPIAYT TNFLKDNELAVIKNNSEYIETTSKAYTDGKINIDHSGGYVAQFNISWDEVNYDLEIV GGWECEKHSOPWQVLVASRGRAVCGGVLVHPOWVLTAAHCIRNKSVILLGRHSL FHPEDTGOVFOVSHSFPHPLYDMSLLKNRFLRPGDDSSHDLMLLRLSEPAELTDAV KVMDLPTOEPALGTTCYASGWGSIEPEEFLTPKKLOCVDLHVISNDVCAOVHPOKV TKFMLCAGRWTGGKSTCSGDSGGPLVCYGVLOGITSWGSEPCALPERPSLYTKVV HYRKWIKDTIVANP (PSA sequence is underlined) (SEQ ID NO: 13). In another embodiment, the tLLO-PSA fusion protein is a homologue of SEQ ID NO: 13. In another embodiment, the tLLO-PSA fusion protein is a variant of SEQ ID NO: 13. In another embodiment, the tLLO-PSA fusion protein is an isomer of SEQ ID NO: 13. In another embodiment, the tLLO-PSA fusion protein is a fragment of SEQ ID NO: 13.

In one embodiment, the Her2-neu chimeric protein, harbors two of the extracellular and one intracellular fragments of Her2/neu antigen showing clusters of MHC-class I epitopes of the oncogene, where, in another embodiment, the chimeric protein, harbors 3 H2Dq and at least 17 of the mapped human MHC-class I epitopes of the Her2/neu antigen (fragments EC1, EC2, and IC1) as described in U.S. patent application Ser. No. 12/945,386, which is incorporated by reference herein in its entirety. In another embodiment, the Her2-neu chimeric protein is fused to the first 441 amino acids of the Listeria-monocytogenes listeriolysin O (LLO) protein and expressed and secreted by the Listeria monocytogenes attenuated auxotrophic strain LmddA. In another embodiment, the Her2-neu chimeric protein is fused to the first 441 amino acids of the Listeria-monocytogenes listeriolysin O (LLO) protein and is expressed from the chromosome of a recombinant Listeria disclosed herein, while an additional antigen is expressed from a plasmid present within the recombinant Listeria disclosed herein. In another embodiment, the Her2-neu chimeric protein is fused to the first 441 amino acids of the Listeria-monocytogenes listeriolysin O (LLO) protein and is expressed from a plasmid of a recombinant Listeria disclosed herein, while an additional antigen is expressed from the chromosome of the recombinant Listeria disclosed herein. In another embodiment, a recombinant Listeria disclosed herein is a Listeria monocytogenes attenuated auxotrophic strain LmddA.

In one embodiment, a chimeric HER2 protein is encoded by the following nucleic acid sequence set forth in SEQ ID NO:14

acccacctggacatgctccgccacctctaccagggctgccaggtggtgcagggaaacctggaactcacctacctgcc caccaatgccagcctgtccttcctgcaggatatccaggaggtgcagggctacgtgctcatcgctcacaaccaagtgaggcaggt cccactgcagaggctgcggattgtgcgaggcacccagctctttgaggacaactatgccctggccgtgctagacaatggagaccc gctgaacaataccacccctgtcacaggggcctccccaggaggcctgcgggagctgcagcttcgaagcctcacagagatcttga aaggaggggtcttgatccagcggaacccccagctctgctaccaggacacgattttgtggaagaatatccaggagtttgctggctg caagaagatctttgggagcctggcatttctgccggagagctttgatggggacccagcctccaacactgccccgctccagccaga gcagctccaagtgtttgagactctggaagagatcacaggttacctatacatctcagcatggccggacagcctgcctgacctcagc gtcttccagaacctgcaagtaatccggggacgaattctgcacaatggcgcctactcgctgaccctgcaagggctgggcatcagct ggctggggctgcgctcactgagggaactgggcagtggactggccctcatccaccataacacccacctctgcttcgtgcacacgg tgccctgggaccagctctttcggaacccgcaccaagctctgctccacactgccaaccggccagaggacgagtgtgtgggcgag ggcctggcctgccaccagctgtgcgcccgagggcagcagaagatccggaagtacacgatgcggagactgctgcaggaaacg gagctggtggagccgctgacacctagcggagcgatgcccaaccaggcgcagatgcggatcctgaaagagacggagctgagg aaggtgaaggtgcttggatctggcgcttttggcacagtctacaagggcatctggatccctgatggggagaatgtgaaaattccagt ggccatcaaagtgttgagggaaaacacatcccccaaagccaacaaagaaatcttagacgaagcatacgtgatggctggtgtggg ctccccatatgtctcccgccttctgggcatctgcctgacatccacggtgcagctggtgacacagcttatgccctatggctgcctctta gac (SEQ ID NO: 14). In another embodiment, the cHER2 protein is encoded by a homologue of SEQ ID NO: 14. In another embodiment, the cHER2 protein is encoded by a variant of SEQ ID NO: 14. In another embodiment, the cHER2 protein is encoded by an isomer of SEQ ID NO: 14. In another embodiment, the cHER2 protein is encoded by a fragment of SEQ ID NO: 14.

In one embodiment, a chimeric HER2 protein comprises the sequence:

T H L D M L R H L Y Q G C Q V V Q G N L E L T Y L P T N A S L S F L Q D I Q E V Q G Y V L I A H N Q V R Q V P L Q R L R I V R G T Q L F E D N Y A L A V L D N G D P L N N T T P V T G A S P G G L R E L Q L R S L T E I L K G G V L I Q R N P Q L C Y Q D T I L W K N I Q E F A G C K K I F G S L A F L P E S F D G D P A S N T A P L Q P E Q L Q V F E T L E E I T G Y L Y I S A W P D S L P D L S V F Q N L Q V I R G R I L H N G A Y S L T L Q G L G I S W L G L R S L R E L G S G L A L I H H N T H L C F V H T V P W D Q L F R N P H Q A L L H T A N R P E D E C V G E G L A C H Q L C A R G Q Q K I R K Y T M R R L L Q E T E L V E P L T P S G A M P N Q A Q M R I L K E T E L R K V K V L G S G A F G T V Y K G I W I P D G E N V K I P V A I K V L R E N T S P K A N K E I L D E A Y V M A G V G S P Y V S R L L G I C L T S T V Q L V T Q L M P Y G C L L D (SEQ ID NO: 15). In another embodiment, the cHER2 protein is a homologue of SEQ ID NO: 15. In another embodiment, the cHER2 protein is a variant of SEQ ID NO: 15. In another embodiment, the cHER2 protein is an isomer of SEQ ID NO: 15. In another embodiment, the cHER2 protein is a fragment of SEQ ID NO: 15.

In one embodiment, the Her2 chimeric protein or fragment thereof of the methods and compositions provided herein does not include a signal sequence thereof. In another embodiment, omission of the signal sequence enables the Her2 fragment to be successfully expressed in Listeria, due the high hydrophobicity of the signal sequence. Each possibility represents a separate embodiment of the present disclosure.

In another embodiment, the fragment of a Her2 chimeric protein of methods and compositions of the present disclosure does not include a transmembrane domain (TM) thereof. In one embodiment, omission of the TM enables the Her-2 fragment to be successfully expressed in Listeria, due the high hydrophobicity of the TM.

In one embodiment, a recombinant polypeptide disclosed herein comprising a truncated LLO fused to a cHER2 protein disclosed herein is encoded by a sequence comprising:

ATGAAAAAAATAATGCTAGTTTTTATTACACTTATATTAGTTAGTCTA CCAATTGCGCAACAAACTGAAGCAAAGGATGCATCTGCATTCAATAAAGAAAA TTCAATTTCATCCATGGCACCACCAGCATCTCCGCCTGCAAGTCCTAAGACGCC AATCGAAAAGAAACACGCGGATGAAATCGATAAGTATACAAGGATTGGATT ACAATAAAAACAATGTATTAGTATACCACGGAGATGCAGTGACAAATGTGCCG CCAAGAAAAGGTTACAAAGATGGAAATGAATATATTGTTGTGGAGAAAAAGAA GAAATCCATCAATCAAAATAATGCAGACATTCAAGTTGTGAATGCAATTTCGAG CCTAACCTATCCAGGTGCTCTCGTAAAAGCGAATTCGGAATTAGTAGAAAATCA ACCAGATGTTCTCCCTGTAAAACGTGATTCATTAACACTCAGCATTGATTTGCCA GGTATGACTAATCAAGACAATAAAATAGTTGTAAAAAATGCCACTAAATCAAA CGTTAACAACGCAGTAAATACATTAGTGGAAAGATGGAATGAAAAATATGCTC AAGCTTATCCAAATGTAAGTGCAAAAATTGATTATGATGACGAAATGGCTTACA GTGAATCACAATTAATTGCGAAATTTGGTACAGCATTTAAAGCTGTAAATAATA GCTTGAATGTAAACTTCGGCGCAATCAGTGAAGGGAAAATGCAAGAAGAAGTC ATTAGTTTTAAACAAATTTACTATAACGTGAATGTTAATGAACCTACAAGACCT TCCAGATTTTTCGGCAAAGCTGTTACTAAAGAGCAGTTGCAAGCGCTTGGAGTG AATGCAGAAAATCCTCCTGCATATATCTCAAGTGTGGCGTATGGCCGTCAAGTT TATTTGAAATTATCAACTAATTCCCATAGTACTAAAGTAAAAGCTGCTTTTGATG CTGCCGTAAGCGGAAAATCTGTCTCAGGTGATGTAGAACTAACAAATATCATCA AAAATTCTTCCTTCAAAGCCGTAATTTACGGAGGTTCCGCAAAAGATGAAGTTC AAATCATCGACGGCAACCTCGGAGACTTACGCGATATTTTGAAAAAAGGCGCT ACTTTTAATCGAGAAACACCAGGAGTTCCCATTGCTTATACAACAAACTTCCTA AAAGACAATGAATTAGCTGTTATTAAAAACAACTCAGAATATATTGAAACAACT TCAAAAGCTTATACAGATGGAAAAATTAACATCGATCACTCTGGAGGATACGTT GCTCAATTCAACATTTCTTGGGATGAAGTAAATTATGATctcgagacccacctggacatgctc cgccacctctaccagggctgccaggtggtgcagggaaacctggaactcacctacctgcccaccaatgccagcctgtccttcctgc aggatatccaggaggtgcagggctacgtgctcatcgctcacaaccaagtgaggcaggtcccactgcagaggctgcggattgtgc gaggcacccagctctttgaggacaactatgccctggccgtgctagacaatggagacccgctgaacaataccacccctgtcacag gggcctccccaggaggcctgcgggagctgcagcttcgaagcctcacagagatcttgaaaggaggggtcttgatccagcggaac ccccagctctgctaccaggacacgattttgtggaagaatatccaggagtttgctggctgcaagaagatctttgggagcctggcattt ctgccggagagctttgatggggacccagcctccaacactgccccgctccagccagagcagctccaagtgtttgagactctggaa gagatcacaggttacctatacatctcagcatggccggacagcctgcctgacctcagcgtcttccagaacctgcaagtaatccggg gacgaattctgcacaatggcgcctactcgctgaccctgcaagggctgggcatcagctggctggggctgcgctcactgagggaac tgggcagtggactggccctcatccaccataacacccacctctgcttcgtgcacacggtgccctgggaccagctctttcggaacccg caccaagctctgctccacactgccaaccggccagaggacgagtgtgtgggcgagggcctggcctgccaccagctgtgcgcccg agggcagcagaagatccggaagtacacgatgcggagactgctgcaggaaacggagctggtggagccgctgacacctagcg gagcgatgcccaaccaggcgcagatgcggatcctgaaagagacggagctgaggaaggtgaaggtgcttggatctggcgctttt ggcacagtctacaagggcatctggatccctgatggggagaatgtgaaaattccagtggccatcaaagtgttgagggaaaacac atcccccaaagccaacaaagaaatcttagacgaagcatacgtgatggctggtgtgggctccccatatgtctcccgccttctgggc atctgcctgacatccacggtgcagctggtgacacagcttatgccctatggctgcctcttagac (SEQ ID NO: 16). In another embodiment, the fusion protein is encoded by a homologue of SEQ ID NO: 16. In another embodiment, the fusion protein is encoded by a variant of SEQ ID NO: 16. In another embodiment, the fusion protein is encoded by an isomer of SEQ ID NO: 16.

In another embodiment, a recombinant polypeptide disclosed herein comprising a truncated LLO fused to a cHER2 protein disclosed herein comprises the following sequence:

(SEQ ID NO: 17)
MKKIMLVFITLILVSLPIAQQTEAKDASAFNKENSISSMAPPASPPASPK
TPIEKKHADEIDKYIQGLDYNKNNVLVYHGDAVTNVPPRKGYKDGNEYIV
VEKKKKSINQNNADIQVVNAISSLTYPGALVKANSELVENQPDVLPVKRD
SLTLSIDLPGMTNQDNKIVVKNATKSNVNNAVNTLVERWNEKYAQAYPNV
SAKIDYDDEMAYSESQLIAKFGTAFKAVNNSLNVNFGAISEGKMQEEVIS
FKQIYYNVNVNEPTRPSRFFGKAVTKEQLQALGVNAENPPAYISSVAYGR
QVYLKLSTNSHSTKVKAAFDAAVSGKSVSGDVELTNIIKNSSFKAVIYGG
SAKDEVQIIDGNLGDLRDILKKGATFNRETPGVPIAYTTNFLKDNELAVI
KNNSEYIETTSKAYTDGKINIDHSGGYVAQFNISWDEVNYDL E T H L
D M L R H L Y Q G C Q V V Q G N L E L T Y L P T N
A S L S F L Q D I Q E V Q G Y V L I A H N Q V R Q
V P L Q R L R I V R G T Q L F E D N Y A L A V L D
N G D P L N N T T P V T G A S P G G L R E L Q L R
S L T E I L K G G V L I Q R N P Q L C Y Q D T I L
W K N I Q E F A G C K K I F G S L A F L P E S F D
G D P A S N T A P L Q P E Q L Q V F E T L E E I T
G Y L Y I S A W P D S L P D L S V F Q N L Q V I R
G R I L H N G A Y S L T L Q G L G I S W L G L R S
L R E L G S G L A L I H H N T H L C F V H T V P W
D Q L F R N P H Q A L L H T A N R P E D E C V G E
G L A C H Q L C A R G Q Q K I R K Y T M R R L L Q
E T E L V E P L T P S G A M P N Q A Q M R I L K E
T E L R K V K V L G S G A F G T V Y K G I W I P D
G E N V K I P V A I K V L R E N T S P K A N K E I
L D E A Y V M A G V G S P Y V S R L L G I C L T S
T V Q L V T Q L M P Y G C L L D
(cHER2 sequence underlined).

In another embodiment, the tLLO-cHER2 fusion protein is a homologue of SEQ ID NO: 17. In another embodiment, the tLLO-cHER2 fusion protein is a variant of SEQ ID NO: 17. In another embodiment, the tLLO-cHER2 fusion protein is an isomer of SEQ ID NO: 17. In another embodiment, the tLLO-cHER2 fusion protein is a fragment of SEQ ID NO: 17.

In one embodiment, an antigen disclosed herein is fused to an N-terminal ActA protein. In another embodiment, an N-terminal fragment of an ActA protein utilized in methods and compositions disclosed herein has, in another embodiment, the sequence set forth in SEQ ID NO: 18:

MRAMMVVFITANCITINPDIIFAATDSEDSSLNTDEWEEEKTEEQPSEVN
TGPRYETAREVSSRDIKELEKSNKVRNTNKADLIAMLKEKAEKGPNINNN
NSEQTENAAINEEASGADRPAIQVERRHPGLPSDSAAEIKKRRKAIASSD
SELESLTYPDKPTKVNKKKVAKESVADASESDLDSSMQSADESSPQPLKA
NQQPFFPKVFKKIKDAGKWVRDKIDENPEVKKAIVDKSAGLIDQLLTKKK
SEEVNASDFPPPPTDEELRLALPETPMLLGFNAPATSEPSSFEFPPPPTD
EELRLALPETPMLLGFNAPATSEPSSFEFPPPPTEDELEIIRETASSLDS
SFTRGDLASLRNAINRHSQNFSDFPPIPTEEELNGRGGRP.

In another embodiment, the ActA fragment comprises the sequence set forth in SEQ ID NO: 18. In another embodiment, the ActA fragment is any other ActA fragment known in the art.

In another embodiment, the recombinant nucleotide encoding a fragment of an ActA protein comprises the sequence set forth in SEQ ID NO: 19:

Atgcgtgcgatgatggtggttttcattactgccaattgcattacgattaaccccgacataatatttgcagcgacagatagc gaagattctagtctaaacacagatgaatgggaagaagaaaaaacagaagagcaaccaagcgaggtaaatacgggaccaagat acgaaactgcacgtgaagtaagttcacgtgatattaaagaactagaaaaatcgaataaagtgagaaatacgaacaaagcagacct aatagcaatgttgaaagaaaaagcagaaaaaggtccaaatatcaataataacaacagtgaacaaactgagaatgcggctataaat gaagaggcttcaggagccgaccgaccagctatacaagtggagcgtcgtcatccaggattgccatcggatagcgcagcggaaat taaaaaaagaaggaaagccatagcatcatcggatagtgagcttgaaagccttacttatccggataaaccaacaaaagtaaataag aaaaaagtggcgaaagagtcagttgcggatgcttctgaaagtgacttagattctagcatgcagtcagcagatgagtcttcaccaca acctttaaaagcaaaccaacaaccatttttccctaaagtatttaaaaaaataaaagatgcggggaaatgggtacgtgataaaatcga cgaaaatcctgaagtaaagaaagcgattgttgataaaagtgcagggttaattgaccaattattaaccaaaaagaaaagtgaagagg taaatgcttcggacttcccgccaccacctacggatgaagagttaagacttgctttgccagagacaccaatgcttcttggttttaatgct cctgctacatcagaaccgagctcattcgaatttccaccaccacctacggatgaagagttaagacttgctttgccagagacgccaat gcttcttggttttaatgctcctgctacatcggaaccgagctcgttcgaatttccaccgcctccaacagaagatgaactagaaatcatc cgggaaacagcatcctcgctagattctagttttacaagaggggatttagctagtttgagaaatgctattaatcgccatagtcaaaattt ctctgatttcccaccaatcccaacagaagaagagttgaacgggagaggcggtagacca. In another embodiment, the recombinant nucleotide has the sequence set forth in SEQ ID NO: 19. In another embodiment, the recombinant nucleotide comprises any other sequence that encodes a fragment of an ActA protein.

In another embodiment, a truncated ActA sequence disclosed herein is further fused to an hly signal peptide at the N-terminus. In another embodiment, the truncated ActA fused to hly signal peptide is set forth in SEQ ID NO: 20:

M K K I M L V F I T L I L V S L P I A Q Q T E A S R A T D S E D S S L N T D E W E E E K T E E Q P S E V N T G P R Y E T A R E V S S R D I E E L E K S N K V K N T N K A D L I A M L K A K A E K G P N N N N N N G E Q T G N V A I N E E A S G V D R P T L Q V E R R H P G L S S D S A A E I K K R R K A I A S S D S E L E S L T Y P D K P T K A N K R K V A K E S V V D A S E S D L D S S M Q S A D E S T P Q P L K A N Q K P F F P K V F K K I K D A G K W V R D K. In another embodiment, a truncated ActA as set forth in SEQ ID NO: 20 is referred to as “LA229”.

In another embodiment, a truncated ActA fused to hly signal peptide is encoded by a sequence comprising SEQ ID NO: 21:

Atgaaaaaaataatgctagtttttattacacttatattagttagtctaccaattgcgcaacaaactgaagcatctagagcga cagatagcgaagattccagtctaaacacagatgaatgggaagaagaaaaaacagaagagcagccaagcgaggtaaatacggg accaagatacgaaactgcacgtgaagtaagttcacgtgatattgaggaactagaaaaatcgaataaagtgaaaaatacgaacaaa gcagacctaatagcaatgttgaaagcaaaagcagagaaaggtccgaataacaataataacaacggtgagcaaacaggaaatgt ggctataaatgaagaggcttcaggagtcgaccgaccaactctgcaagtggagcgtcgtcatccaggtctgtcatcggatagcgca gcggaaattaaaaaaagaagaaaagccatagcgtcgtcggatagtgagcttgaaagccttacttatccagataaaccaacaaaag caaataagagaaaagtggcgaaagagtcagttgtggatgcttctgaaagtgacttagattctagcatgcagtcagcagacgagtct acaccacaacctttaaaagcaaatcaaaaaccatttttccctaaagtatttaaaaaaataaaagatgcggggaaatgggtacgtgat aaa (SEQ ID NO: 21). In another embodiment, SEQ ID NO: 39 comprises a sequence encoding a linker region (see bold, italic text) that is used to create a unique restriction enzyme site for XbaI so that different polypeptides, heterologous antigens, etc. can be cloned after the signal sequence. Hence, it will be appreciated by a skilled artisan that signal peptidases act on the sequences before the linker region to cleave signal peptide. In another embodiment, a truncated ActA protein is a fragment of an ActA protein. In another embodiment, the truncated ActA protein is an N-terminal fragment of an ActA protein. In another embodiment, the terms “truncated ActA,” “N-terminal ActA fragment” or “ΔActA” are used interchangeably herein and refer to a fragment of ActA that comprises a PEST domain. In another embodiment, the terms refer to an ActA fragment that comprises a PEST sequence. In another embodiment, the terms refer to an immunogenic fragment of the ActA protein.

Thus, fusion of an antigen to other LM PEST sequences and PEST sequences derived from other prokaryotic organisms will also enhance immunogenicity of the antigen. The PEST AA sequence has, in another embodiment, a sequence selected from SEQ ID NO: 22-27. In another embodiment, the PEST sequence is a PEST sequence from the LM ActA protein. In another embodiment, the PEST sequence is KTEEQPSEVNTGPR (SEQ ID NO: 22), KASVTDTSEGDLDSSMQSADESTPQPLK (SEQ ID NO: 23), KNEEVNASDFPPPPTDEELR (SEQ ID NO: 24), or RGGIPTSEEFSSLNSGDFTDDENSETTEEEIDR (SEQ ID NO: 25). In another embodiment, the PEST-like sequence is from Streptolysin O protein of Streptococcus sp. In another embodiment, the PEST-like sequence is from Streptococcus pyogenes Streptolysin O, e.g. KQNTASTETTTTNEQPK (SEQ ID NO: 26) at AA 35-51. In another embodiment, the PEST sequence is from Streptococcus equisimilis Streptolysin O, e.g. KQNTANTETTTTNEQPK (SEQ ID NO: 27) at AA 38-54. In another embodiment, the PEST sequence is another PEST AA sequence derived from a prokaryotic organism. In another embodiment, the PEST sequence is any other PEST sequence known in the art.

PEST sequences of other prokaryotic organism can be identified in accordance with methods such as described by, for example Rechsteiner and Rogers (1996, Trends Biochem. Sci. 21:267-271) for LM. Alternatively, PEST AA sequences from other prokaryotic organisms can also be identified based by this method. Other prokaryotic organisms wherein PEST AA sequences would be expected to include, but are not limited to, other Listeria species. In another embodiment, the PEST sequence is embedded within the antigenic protein. Thus, in another embodiment, “fusion” refers to an antigenic protein comprising both the antigen and the PEST amino acid sequence either linked at one end of the antigen or embedded within the antigen.

In another embodiment, the PEST sequence is identified using any other method or algorithm known in the art, e.g the CaSPredictor (Garay-Malpartida H M, Occhiucci J M, Alves J, Belizario J E. Bioinformatics. 2005 June; 21 Suppl 1:i169-76). In another embodiment, the following method is used:

A PEST index is calculated for each 30-35 AA stretch by assigning a value of 1 to the amino acids Ser, Thr, Pro, Glu, Asp, Asn, or Gln. The coefficient value (CV) for each of the PEST residue is 1 and for each of the other AA (non-PEST) is 0.

In one embodiment, the terms “PEST-like sequence,” “PEST-like amino acid sequence”, “PEST amino acid sequence” and “PEST-sequence” are used interchangeably herein.

In another embodiment, the LLO protein, ActA protein, or fragment thereof disclosed herein need not be that which is set forth exactly in the sequences set forth herein, but rather other alterations, modifications, or changes can be made that retain the functional characteristics of an LLO or ActA protein fused to an antigen as set forth elsewhere herein. In another embodiment, the present disclosure utilizes an analog of an LLO protein, ActA protein, or fragment thereof. Analogs differ, in another embodiment, from naturally occurring proteins or peptides by conservative AA sequence differences or by modifications which do not affect sequence, or by both.

In another embodiment, “homology” refers to identity to an LLO sequence disclosed herein of greater than 70%. In another embodiment, “homology” refers to identity to an LLO sequence disclosed herein of greater than 72%. In another embodiment, “homology” refers to identity to an LLO sequence disclosed herein of greater than 75%. In another embodiment, “homology” refers to identity to an LLO sequence disclosed herein of greater than 78%. In another embodiment, “homology” refers to identity to an LLO sequence disclosed herein of greater than 80%. In another embodiment, “homology” refers to identity to an LLO sequence disclosed herein of greater than 82%. In another embodiment, “homology” refers to identity to an LLO sequence disclosed herein of greater than 83%. In another embodiment, “homology” refers to identity to an LLO sequence disclosed herein of greater than 85%. In another embodiment, “homology” refers to identity to an LLO sequence disclosed herein of greater than 87%. In another embodiment, “homology” refers to identity to an LLO sequence disclosed herein of greater than 88%. In another embodiment, “homology” refers to identity to an LLO sequence disclosed herein of greater than 90%. In another embodiment, “homology” refers to identity to an LLO sequence disclosed herein of greater than 92%. In another embodiment, “homology” refers to identity to an LLO sequence disclosed herein of greater than 93%. In another embodiment, “homology” refers to identity to an LLO sequence disclosed herein of greater than 95%. In another embodiment, “homology” refers to identity to an LLO sequence disclosed herein of greater than 96%. In another embodiment, “homology” refers to identity to an LLO sequence disclosed herein of greater than 97%. In another embodiment, “homology” refers to identity to an LLO sequence disclosed herein of greater than 98%. In another embodiment, “homology” refers to identity to an LLO sequence disclosed herein of greater than 99%. In another embodiment, “homology” refers to identity to an LLO sequence disclosed herein of 100%.

In another embodiment, “homology” refers to identity to a PSA or KLK3 sequence disclosed herein of greater than 70%. In another embodiment, “homology” refers to identity to a PSA sequence of greater than 72%. In another embodiment, “homology” refers to identity to a PSA sequence of greater than 75%. In another embodiment, “homology” refers to identity to a PSA sequence of greater than 78%. In another embodiment, “homology” refers to identity to a PSA sequence of greater than 80%. In another embodiment, “homology” refers to identity to a PSA sequence of greater than 82%. In another embodiment, “homology” refers to identity to a PSA sequence of greater than 83%. In another embodiment, “homology” refers to identity to a PSA sequence of greater than 85%. In another embodiment, “homology” refers to identity to a PSA sequence of greater than 87%. In another embodiment, “homology” refers to identity to a PSA sequence of greater than 88%. In another embodiment, “homology” refers to identity to a PSA sequence of greater than 90%. In another embodiment, “homology” refers to identity to a PSA sequence of greater than 92%. In another embodiment, “homology” refers to identity to a PSA sequence of greater than 93%. In another embodiment, “homology” refers to identity to a PSA sequence of greater than 95%. In another embodiment, “homology” refers to identity to a PSA sequence of greater than 96%. In another embodiment, “homology” refers to identity to a PSA sequence of greater than 97%. In another embodiment, “homology” refers to identity to a PSA sequence of greater than 98%. In another embodiment, “homology” refers to identity to a PSA sequence of greater than 99%. In another embodiment, “homology” refers to identity to a PSA sequence of 100%.

In another embodiment, “homology” refers to identity to a chimeric HER2 sequence of greater than 70%. In another embodiment, “homology” refers to identity to a cHER2 sequence disclosed herein of greater than 72%. In another embodiment, “homology” refers to identity to a cHER2 sequence disclosed herein of greater than 75%. In another embodiment, “homology” refers to identity to a cHER2 sequence disclosed herein of greater than 78%. In another embodiment, “homology” refers to identity to a cHER2 sequence disclosed herein of greater than 80%. In another embodiment, “homology” refers to identity to a cHER2 sequence disclosed herein of greater than 82%. In another embodiment, “homology” refers to identity to a cHER2 sequence disclosed herein of greater than 83%. In another embodiment, “homology” refers to identity to a cHER2 sequence disclosed herein of greater than 85%. In another embodiment, “homology” refers to identity to a cHER2 sequence disclosed herein of greater than 87%. In another embodiment, “homology” refers to identity to a cHER2 sequence disclosed herein of greater than 88%. In another embodiment, “homology” refers to identity to a cHER2 sequence disclosed herein of greater than 90%. In another embodiment, “homology” refers to identity to a cHER2 sequence disclosed herein of greater than 92%. In another embodiment, “homology” refers to identity to a cHER2 sequence disclosed herein of greater than 93%. In another embodiment, “homology” refers to identity to a cHER2 sequence disclosed herein of greater than 95%. In another embodiment, “homology” refers to identity to a cHER2 sequence disclosed herein of greater than 96%. In another embodiment, “homology” refers to identity to a cHER2 sequence disclosed herein of greater than 97%. In another embodiment, “homology” refers to identity to a cHER2 sequence disclosed herein of greater than 98%. In another embodiment, “homology” refers to identity to a cHER2 sequence disclosed herein of greater than 99%. In another embodiment, “homology” refers to identity to a cHER2 sequence disclosed herein of 100%.

In another embodiment, “homology” refers to identity to a PEST sequence disclosed herein or to an ActA sequence disclosed herein of greater than 70%. In another embodiment, “homology” refers to identity to a PEST sequence disclosed herein or to an ActA sequence disclosed herein of greater than 72%. In another embodiment, “homology” refers to identity to a PEST sequence disclosed herein or to an ActA sequence disclosed herein of greater than 75%. In another embodiment, “homology” refers to identity to a PEST sequence disclosed herein or to an ActA sequence disclosed herein of greater than 78%. In another embodiment, “homology” refers to identity to a PEST sequence disclosed herein or to an ActA sequence disclosed herein of greater than 80%. In another embodiment, “homology” refers to identity to a PEST sequence disclosed herein or to an ActA sequence disclosed herein of greater than 82%. In another embodiment, “homology” refers to identity to a PEST sequence disclosed herein or to an ActA sequence disclosed herein of greater than 83%. In another embodiment, “homology” refers to identity to a PEST sequence disclosed herein or to an ActA sequence disclosed herein of greater than 85%. In another embodiment, “homology” refers to identity to a PEST sequence disclosed herein or to an ActA sequence disclosed herein of greater than 87%. In another embodiment, “homology” refers to identity to a PEST sequence disclosed herein or to an ActA sequence disclosed herein of greater than 88%. In another embodiment, “homology” refers to identity to a PEST sequence disclosed herein or to an ActA sequence disclosed herein of greater than 90%. In another embodiment, “homology” refers to identity to a PEST sequence disclosed herein or to an ActA sequence disclosed herein of greater than 92%. In another embodiment, “homology” refers to identity to a PEST sequence disclosed herein or to an ActA sequence disclosed herein of greater than 93%. In another embodiment, “homology” refers to identity to a PEST sequence disclosed herein or to an ActA sequence disclosed herein of greater than 95%. In another embodiment, “homology” refers to identity to a PEST sequence disclosed herein or to an ActA sequence disclosed herein of greater than 96%. In another embodiment, “homology” refers to identity to a PEST sequence disclosed herein or to an ActA sequence disclosed herein of greater than 97%. In another embodiment, “homology” refers to identity to a PEST sequence disclosed herein or to an ActA sequence disclosed herein of greater than 98%. In another embodiment, “homology” refers to identity to a PEST sequence disclosed herein or to an ActA sequence disclosed herein of greater than 99%. In another embodiment, “homology” refers to identity to a PEST sequence disclosed herein or to an ActA sequence disclosed herein of 100%.

Protein and/or peptide homology for any AA sequence listed herein is determined, in one embodiment, by methods well described in the art, including immunoblot analysis, or via computer algorithm analysis of AA sequences, utilizing any of a number of software packages available, via established methods. Some of these packages include the FASTA, BLAST, MPsrch or Scanps packages, and employ, in other embodiments, the use of the Smith and Waterman algorithms, and/or global/local or BLOCKS alignments for analysis, for example. Each method of determining homology represents a separate embodiment of the present disclosure.

In another embodiment, the LLO protein, ActA protein, or fragment thereof is attached to anantigen or fragment thereof disclosed herein by chemical conjugation. In another embodiment, glutaraldehyde is used for the conjugation. In another embodiment, the conjugation is performed using any suitable method known in the art. Each possibility represents another embodiment of the present disclosure.

In another embodiment, fusion proteins of the present disclosure are prepared by any suitable method, including, for example, cloning and restriction of appropriate sequences or direct chemical synthesis by methods discussed below. In another embodiment, subsequences are cloned and the appropriate subsequences cleaved using appropriate restriction enzymes. The fragments are then ligated, in another embodiment, to produce the desired DNA sequence. In another embodiment, DNA encoding the fusion protein is produced using DNA amplification methods, for example polymerase chain reaction (PCR). First, the segments of the native DNA on either side of the new terminus are amplified separately. The 5′ end of the one amplified sequence encodes the peptide linker, while the 3′ end of the other amplified sequence also encodes the peptide linker. Since the 5′ end of the first fragment is complementary to the 3′ end of the second fragment, the two fragments (after partial purification, e.g. on LMP agarose) can be used as an overlapping template in a third PCR reaction. The amplified sequence will contain codons, the segment on the carboxy side of the opening site (now forming the amino sequence), the linker, and the sequence on the amino side of the opening site (now forming the carboxyl sequence). The insert is then ligated into a plasmid.

In another embodiment, the LLO protein, ActA protein, or fragment thereof and the antigen or fragment thereof disclosed herein are conjugated by a means known to those of skill in the art. In another embodiment, the antigen or fragment thereof is conjugated, either directly or through a linker (spacer), to the ActA protein or LLO protein. In another embodiment, the chimeric molecule is recombinantly expressed as a single-chain fusion protein.

In another embodiment, the present disclosure provides a kit comprising immunotherapy, an applicator, and instructional material that describes use of the methods of the disclosure. Although model kits are described below, the contents of other useful kits will be apparent to the skilled artisan in light of the present disclosure.

In one embodiment, the singular forms of words such as “a,” “an,” and “the,” include their corresponding plural references unless the context clearly dictates otherwise.

Throughout this application, various embodiments of this disclosure may be presented in a range format. It should be understood that the description in range format is merely for convenience and brevity and should not be construed as an inflexible limitation on the scope of the disclosure. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

It will be appreciated by a skilled artisan that the term “About” when used to modify a numerically defined parameter may encompass variation of the parameter in quantitative terms plus or minus 5%, or in another embodiment plus or minus 10%, or in another embodiment plus or minus 15%, or in another embodiment plus or minus 20% of stated numerical value for that parameter.

It is to be understood by the skilled artisan that the term “subject” can encompass a mammal including an adult human or a human child, teenager or adolescent in need of therapy for, or susceptible to, a condition or its sequelae, and also may include non-human mammals such as dogs, cats, pigs, cows, sheep, goats, horses, rats, and mice. It will also be appreciated that the term may encompass livestock. The term “subject” does not exclude an individual that is normal in all respects.

It will be appreciated by the skilled artisan that the term “mammal” for purposes of treatment refers to any animal classified as a mammal, including, but not limited to, humans, domestic and farm animals, and zoo, sports, or pet animals, such as canines, including dogs, and horses, cats, cattle, pigs, sheep, etc.

In the following examples, numerous specific details are set forth in order to provide a thorough understanding of the disclosure. However, it will be understood by those skilled in the art that the embodiments of present disclosure may be practiced without these specific details. In other instances, well-known methods, procedures, and components have not been described in detail so as not to obscure the present disclosure. Thus these examples should in no way be construed, as limiting the broad scope of the invention. Moreover, although sub-features may be described herein as separate embodiments which include distinct features, the inventors intend these embodiments to combinable into any combination or configuration as if set forth specifically herein.

### EXPERIMENTAL DETAILS SECTION

### Example 1: Construction of Attenuated Listeria Strain-LmddΔactA and Insertion of the Human klk3 Gene in Frame to the hly Gene in the Lmdd and Lmdda Strains

### Materials and Methods

A recombinant Lm was developed that secretes PSA fused to tLLO (Lm-LLO-PSA), which elicits a potent PSA-specific immune response associated with regression of tumors in a mouse model for prostate cancer, wherein the expression of tLLO-PSA is derived from a plasmid based on pGG55 (Table 1), which confers antibiotic resistance to the vector. We recently developed a new strain for the PSA immunotherapy based on the pADV142 plasmid, which has no antibiotic resistance markers, and referred as LmddA-142 (Table 1). This new strain is 10 times more attenuated than Lm-LLO-PSA. In addition, LmddA-142 was slightly more immunogenic and significantly more efficacious in regressing PSA expressing tumors than the Lm-LLO-PSA.

TABLE 1
Plasmids and strains

| Plasmids | Features |
|---|---|
| pGG55 | pAM401/pGB354 shuttle plasmid with gram(−) |
| | and gram(+) cm resistance, LLO-E7 expression |
| | cassette and a copy of Lm prfA gene |
| pTV3 | Derived from pGG55 by deleting cm genes |
| | and inserting the Lm dal gene |
| pADV119 | Derived from pTV3 by deleting the prfA gene |
| pADV134 | Derived from pADV119 by replacing the Lm dal |
| | gene by the Bacillus dal gene |
| pADV142 | Derived from pADV134 by replacing HPV16 |
| | e7 with klk3 |
| pADV168 | Derived from pADV134 by replacing HPV16 |
| | e7 with hm w-maa₂₁₆₀₋₂₂₅₈ |
| Strains | Genotype |
| 10403S | Wild-type Listeria monocytogenes:: str |
| XFL-7 | 10403S prfA⁽⁻⁾ |
| Lmdd | 10403S dal⁽⁻⁾ dat⁽⁻⁾ |
| LmddA | 10403S dal⁽⁻⁾ dat⁽⁻⁾ actA⁽⁻⁾ |
| LmddA-134 | 10403S dal⁽⁻⁾ dat⁽⁻⁾ actA⁽⁻⁾ pADV134 |
| LmddA-142 | 10403S dal⁽⁻⁾ dat⁽⁻⁾ actA⁽⁻⁾ pADV142 |
| Lmdd-143 | 10403S dal⁽⁻⁾ dat⁽⁻⁾ with klk3 fused to the |
| | hly gene in the chromosome |
| LmddA-143 | 10403S dal⁽⁻⁾ dat⁽⁻⁾ actA⁽⁻⁾ with klk3 fused |
| | to the hly gene in the chromosome |
| LmddA-168 | 10403S dal⁽⁻⁾ dat⁽⁻⁾ actA⁽⁻⁾ pADV168 |
| Lmdd-143/134 | Lmdd-143 pADV134 |
| LmddA-143/134 | LmddA-143 pADV134 |
| Lmdd-143/168 | Lmdd-143 pADV168 |
| LmdAA-143/168 | LmddA-143 pADV168 |

The sequence of the plasmid pAdv142 (6523 bp) was as follows:

(SEQ ID NO: 28)
cggagtgtatactggcttactatgttggcactgatgagggtgtcagtgaagtgcttcatgtggcaggagaaaaaaggctgc
accggtgcgtcagcagaatatgtgatacaggatatattccgcttcctcgctcactgactcgctacgctcggtcgttcgactgcggcgag
cggaaatggcttacgaacggggcggagatttcctggaagatgccaggaagatacttaacagggaagtgagagggccgcggcaaa
gccgtttttccataggctccgcccccctgacaagcatcacgaaatctgacgctcaaatcagtggtggcgaaacccgacaggactataa
agataccaggcgtttccccctggcggctccctcgtgcgctctcctgttcctgcctttcggtttaccggtgtcattccgctgttatggccgcg
tttgtctcattccacgcctgacactcagttccgggtaggcagttcgctccaagctggactgtatgcacgaaccccccgttcagtccgacc
gctgcgccttatccggtaactatcgtcttgagtccaacccggaaagacatgcaaaagcaccactggcagcagccactggtaattgattt
agaggagttagtcttgaagtcatgcgccggttaaggctaaactgaaaggacaagttttggtgactgcgctcctccaagccagttacctc
ggttcaaagagttggtagctcagagaaccttcgaaaaaccgccctgcaaggcggttttttcgttttcagagcaagagattacgcgcaga
ccaaaacgatctcaagaagatcatcttattaatcagataaaatatttctagccctcctttgattagtatattcctatcttaaagttacttttatgtg
gaggcattaacatttgttaatgacgtcaaaaggatagcaagactagaataaagctataaagcaagcatataatattgcgtttcatctttaga
agcgaatttcgccaatattataattatcaaaagagaggggtggcaaacggtatttggcattattaggttaaaaaatgtagaaggagagtg
aaacccatgaaaaaaataatgctagtttttattacacttatattagttagtctaccaattgcgcaacaaactgaagcaaaggatgcatctgc
attcaataaagaaaattcaatttcatccatggcaccaccagcatctccgcctgcaagtcctaagacgccaatcgaaaagaaacacgcg
gatgaaatcgataagtatatacaaggattggattacaataaaaacaatgtattagtataccacggagatgcagtgacaaatgtgccgcca
agaaaaggttacaaagatggaaatgaatatattgttgtggagaaaaagaagaaatccatcaatcaaaataatgcagacattcaagttgtg
aatgcaatttcgagcctaacctatccaggtgctctcgtaaaagcgaattcggaattagtagaaaatcaaccagatgttctccctgtaaaac
gtgattcattaacactcagcattgatttgccaggtatgactaatcaagacaataaaatagttgtaaaaaatgccactaaatcaaacgttaac
aacgcagtaaatacattagtggaaagatggaatgaaaaatatgctcaagcttatccaaatgtaagtgcaaaaattgattatgatgacgaa
atggcttacagtgaatcacaattaattgcgaaatttggtacagcatttaaagctgtaaataatagcttgaatgtaaacttcggcgcaatcag
tgaagggaaaatgcaagaagaagtcattagttttaaacaaatttactataacgtgaatgttaatgaacctacaagaccttccagatttttcg
gcaaagctgttactaaagagcagttgcaagcgcttggagtgaatgcagaaaatcctcctgcatatatctcaagtgtggcgtatggccgt
caagtttatttgaaattatcaactaattcccatagtactaaagtaaaagctgcttttgatgctgccgtaagcggaaaatctgtctcaggtgat
gtagaactaacaaatatcatcaaaaattcttccttcaaagccgtaatttacggaggttccgcaaaagatgaagttcaaatcatcgacggc
aacctcggagacttacgcgatattttgaaaaaaggcgctacttttaatcgagaaacaccaggagttcccattgcttatacaacaaacttcc
taaaagacaatgaattagctgttattaaaaacaactcagaatatattgaaacaacttcaaaagcttatacagatggaaaaattaacatcgat
cactctggaggatacgttgctcaattcaacatttcttgggatgaagtaaattatgatctcgagattgtgggaggctgggagtgcgagaag
cattcccaaccctggcaggtgcttgtggcctctcgtggcagggcagtctgcggcggtgttctggtgcacccccagtgggtcctcacag
ctgcccactgcatcaggaacaaaagcgtgatcttgctgggtcggcacagcctgtttcatcctgaagacacaggccaggtatttcaggtc
agccacagcttcccacacccgctctacgatatgagcctcctgaagaatcgattcctcaggccaggtgatgactccagccacgacctca
tgctgctccgcctgtcagagcctgccgagctcacggatgctgtgaaggtcatggacctgcccacccaggagccagcactggggacc
acctgctacgcctcaggctggggcagcattgaaccagaggagttcttgaccccaaagaaacttcagtgtgtggacctccatgttatttcc
aatgacgtgtgtgcgcaagttcaccctcagaaggtgaccaagttcatgctgtgtgctggacgctggacagggggcaaaagcacctgc
tcgggtgattctgggggcccacttgtctgttatggtgtgcttcaaggtatcacgtcatggggcagtgaaccatgtgccctgcccgaaag
gccttccctgtacaccaaggtggtgcattaccggaagtggatcaaggacaccatcgtggccaaccccTAAcccgggccactaact
caacgctagtagtggatttaatcccaaatgagccaacagaaccagaaccagaaacagaacaagtaacattggagttagaaatggaag
aagaaaaaagcaatgatttcgtgtgaataatgcacgaaatcattgcttatttttttaaaaagcgatatactagatataacgaaacaacgaac
tgaataaagaatacaaaaaaagagccacgaccagttaaagcctgagaaactttaactgcgagccttaattgattaccaccaatcaattaa
agaagtcgagacccaaaatttggtaaagtatttaattactttattaatcagatacttaaatatctgtaaacccattatatcgggtttttgaggg
gatttcaagtctttaagaagataccaggcaatcaattaagaaaaacttagttgattgccttttttgttgtgattcaactttgatcgtagcttctaa
ctaattaattttcgtaagaaaggagaacagctgaatgaatatcccttttgttgtagaaactgtgcttcatgacggcttgttaaagtacaaattt
aaaaatagtaaaattcgctcaatcactaccaagccaggtaaaagtaaaggggctatttttgcgtatcgctcaaaaaaaagcatgattggc
ggacgtggcgttgttctgacttccgaagaagcgattcacgaaaatcaagatacatttacgcattggacaccaaacgtttatcgttatggta
cgtatgcagacgaaaaccgttcatacactaaaggacattctgaaaacaatttaagacaaatcaataccttctttattgattttgatattcaca
cggaaaaagaaactatttcagcaagcgatattttaacaacagctattgatttaggttttatgcctacgttaattatcaaatctgataaaggtta
tcaagcatattttgttttagaaacgccagtctatgtgacttcaaaatcagaatttaaatctgtcaaagcagccaaaataatctcgcaaaatat
ccgagaatattttggaaagtctttgccagttgatctaacgtgcaatcattttgggattgctcgtataccaagaacggacaatgtagaatttttt
gatcccaattaccgttattctttcaaagaatggcaagattggtctttcaaacaaacagataataagggctttactcgttcaagtctaacggtt
ttaagcggtacagaaggcaaaaaacaagtagatgaaccctggtttaatctcttattgcacgaaacgaaattttcaggagaaaagggttta
gtagggcgcaatagcgttatgtttaccctctctttagcctactttagttcaggctattcaatcgaaacgtgcgaatataatatgtttgagtttaa
taatcgattagatcaacccttagaagaaaaagaagtaatcaaaattgttagaagtgcctattcagaaaactatcaaggggctaataggga
atacattaccattctttgcaaagcttgggtatcaagtgatttaaccagtaaagatttatttgtccgtcaagggtggtttaaattcaagaaaaaa
agaagcgaacgtcaacgtgttcatttgtcagaatggaaagaagatttaatggcttatattagcgaaaaaagcgatgtatacaagccttatt
tagcgacgaccaaaaaagagattagagaagtgctaggcattcctgaacggacattagataaattgctgaaggtactgaaggcgaatc
aggaaattttctttaagattaaaccaggaagaaatggtggcattcaacttgctagtgttaaatcattgttgctatcgatcattaaattaaaaaa
agaagaacgagaaagctatataaaggcgctgacagcttcgtttaatttagaacgtacatttattcaagaaactctaaacaaattggcaga
acgccccaaaacggacccacaactcgatttgtttagctacgatacaggctgaaaataaaacccgcactatgccattacatttatatctatg
atacgtgtttgtttttctttgctggctagcttaattgcttatatttacctgcaataaaggatttcttacttccattatactcccattttccaaaaacat
acggggaacacgggaacttattgtacaggccacctcatagttaatggtttcgagccttcctgcaatctcatccatggaaatatattcatcc
ccctgccggcctattaatgtgacttttgtgcccggcggatattcctgatccagctccaccataaattggtccatgcaaattcggccggcaa
ttttcaggcgttttcccttcacaaggatgtcggtccctttcaattttcggagccagccgtccgcatagcctacaggcaccgtcccgatcca
tgtgtctttttccgctgtgtactcggctccgtagctgacgctctcgccttttctgatcagtttgacatgtgacagtgtcgaatgcagggtaaa
tgccggacgcagctgaaacggtatctcgtccgacatgtcagcagacgggcgaaggccatacatgccgatgccgaatctgactgcatt
aaaaaagccttttttcagccggagtccagcggcgctgttcgcgcagtggaccattagattctttaacggcagcggagcaatcagctcttt
aaagcgctcaaactgcattaagaaatagcctctttctttttcatccgctgtcgcaaaatgggtaaatacccctttgcactttaaacgagggtt
gcggtcaagaattgccatcacgttctgaacttcttcctctgtttttacaccaagtctgttcatccccgtatcgaccttcagatgaaaatgaag
agaaccttttttcgtgtggcgggctgcctcctgaagccattcaacagaataacctgttaaggtcacgtcatactcagcagcgattgccac
atactccgggggaaccgcgccaagcaccaatataggcgccttcaatccctttttgcgcagtgaaatcgcttcatccaaaatggccacg
gccaagcatgaagcacctgcgtcaagagcagcctttgctgtttctgcatcaccatgcccgtaggcgtttgctttcacaactgccatcaag
tggacatgttcaccgatatgttttttcatattgctgacattttcctttatcgcggacaagtcaatttccgcccacgtatctctgtaaaaaggtttt
gtgctcatggaaaactcctctcttttttcagaaaatcccagtacgtaattaagtatttgagaattaattttatattgattaatactaagtttaccca
gttttcacctaaaaaacaaatgatgagataatagctccaaaggctaaagaggactataccaactatttgttaattaa.

This plasmid was sequenced at Genewiz facility from the E. coli strain on Feb. 20, 2008.

The strain Lm dal dat (Lmdd) was attenuated by the irreversible deletion of the virulence factor, ActA. An in-frame deletion of actA in the Lmdaldat (Lmdd) background was constructed to avoid any polar effects on the expression of downstream genes. The Lm dal dat ΔactA contains the first 19 amino acids at the N-terminal and 28 amino acid residues of the C-terminal with a deletion of 591 amino acids of ActA.

The actA deletion mutant was produced by amplifying the chromosomal region corresponding to the upstream (657 bp-oligo's Adv 271/272) and downstream (625 bp-oligo's Adv 273/274) portions of actA and joining by PCR. The sequence of the primers used for this amplification is given in the Table 2. The upstream and downstream DNA regions of actA were cloned in the pNEB193 at the EcoRI/PstI restriction site and from this plasmid, the EcoRI/PstI was further cloned in the temperature sensitive plasmid pKSV7, resulting in AactA/pKSV7 (pAdv120).

TABLE 2
Sequence of primers that was used for the
amplification of DNA sequences upstream and
downstream of actA

| | | SEQ |
|---|---|---|
| | | ID |
| Primer | Sequence | NO: |
| Adv271- | cgGAATTCGGATCCgcgccaaatcattggttgattg | 29 |
| actAF1 | | |
| Adv272- | gcgaGTCGACgtcggggttaatcgtaatgcaattggc | 30 |
| actAR1 | | |
| Adv273- | gcgaGTCGACccatacgacgttaattcttgcaatg | 31 |
| actAF2 | | |
| Adv274- | gataCTGCAGGGATCCttcccttctcggtaatcagtcac | 32 |
| actAR2 | | |

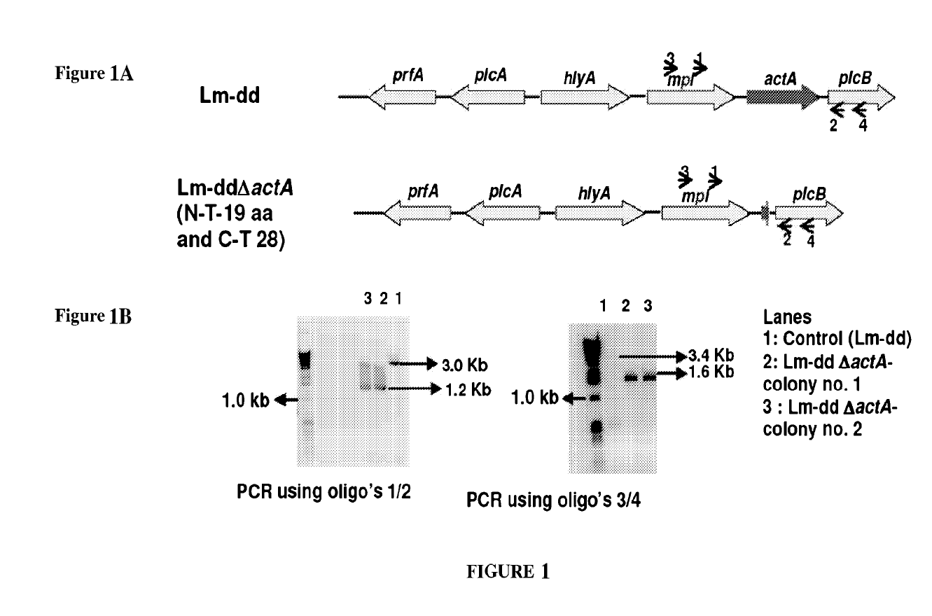

The deletion of the gene from its chromosomal location was verified using primers that bind externally to the actA deletion region, which are shown in FIGS. 1 (A and B) as primer 3 (Adv 305-tgggatggccaagaaattc, SEQ ID NO: 33) and primer 4 (Adv304-ctaccatgtcttccgttgcttg; SEQ ID NO: 34). The PCR analysis was performed on the chromosomal DNA isolated from Lmdd and LmddΔactA. The sizes of the DNA fragments after amplification with two different sets of primer pairs 1/2 and 3/4 in Lmdd chromosomal DNA was expected to be 3.0 Kb and 3.4 Kb. On the other hand, the expected sizes of PCR using the primer pairs 1/2 and 3/4 for the LmddΔactA was 1.2 Kb and 1.6 Kb. Thus, PCR analysis in FIGS. 1 (A and B) confirms that the 1.8 kb region of actA was deleted in the LmddΔactA strain. DNA sequencing was also performed on PCR products to confirm the deletion of actA containing region in the strain, LmddΔactA.

### Example 2: Construction of the Antibiotic-Independent Episomal Expression System for Antigen Delivery by Lm Vectors

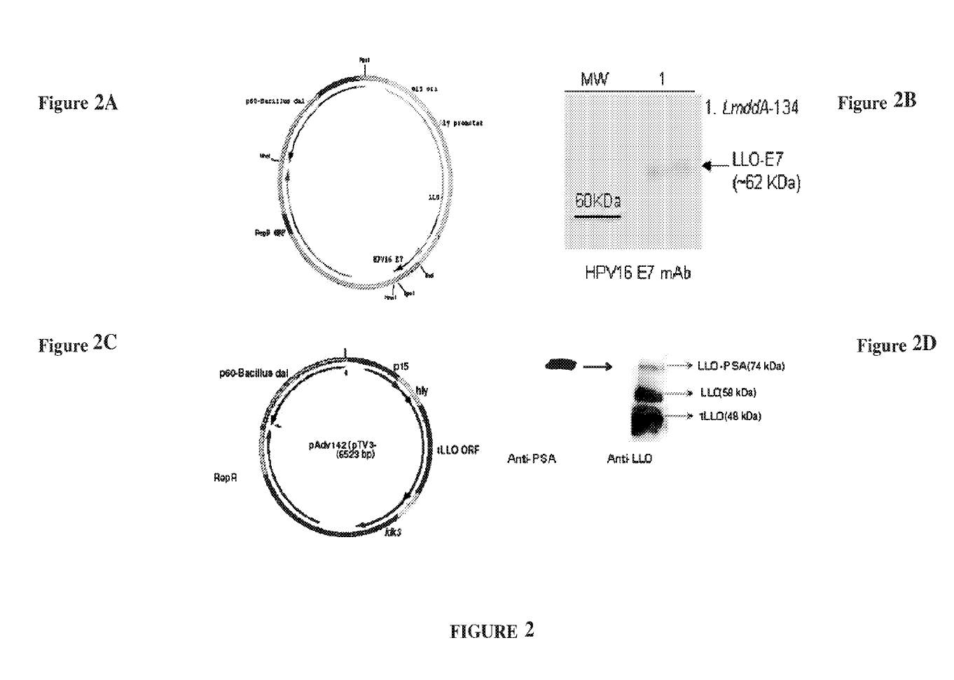

The antibiotic-independent episomal expression system for antigen delivery by Lm vectors (pAdv142) is the next generation of the antibiotic-free plasmid pTV3 (Verch et al., Infect Immun, 2004. 72(11):6418-25, incorporated herein by reference). The gene for virulence gene transcription activator, prfA was deleted from pTV3 since Listeria strain Lmdd contains a copy of prfA gene in the chromosome. Additionally, the cassette for p60-Listeria dal at the NheI/PacI restriction site was replaced by p60-Bacillus subtilis dal resulting in plasmid pAdv134 (FIG. 2A). The similarity of the Listeria and Bacillus dal genes is ˜30%, virtually eliminating the chance of recombination between the plasmid and the remaining fragment of the dal gene in the Lmdd chromosome. The plasmid pAdv134 contained the antigen expression cassette tLLO-E7. The LmddA strain was transformed with the pADV134 plasmid and expression of the LLO-E7 protein from selected clones confirmed by Western blot (FIG. 2B). The Lmdd system derived from the 10403S wild-type strain lacks antibiotic resistance markers, except for the Lmdd streptomycin resistance.

Further, pAdv134 was restricted with XhoI/XmaI to clone human PSA, klk3 resulting in the plasmid, pAdv142. The new plasmid, pAdv142 (FIG. 2C, Table 1) contains Bacillus dal (B-Dal) under the control of Listeria p60 promoter. The shuttle plasmid, pAdv142 complemented the growth of both E. coli ala drx MB2159 as well as Listeria monocytogenes strain Lmdd in the absence of exogenous D-alanine. The antigen expression cassette in the plasmid pAdv142 consists of hly promoter and LLO-PSA fusion protein (FIG. 2C).

The plasmid pAdv142 was transformed to the Listeria background strains, LmddactA strain resulting in Lm-ddA-LLO-PSA. The expression and secretion of LLO-PSA fusion protein by the strain, Lm-ddA-LLO-PSA was confirmed by Western Blot using anti-LLO and anti-PSA antibody (FIG. 2D). There was stable expression and secretion of LLO-PSA fusion protein by the strain, Lm-ddA-LLO-PSA after two in vivo passages.

### Example 3: In Vitro and In Vivo Stability of the Strain LmddA-LLO-PSA

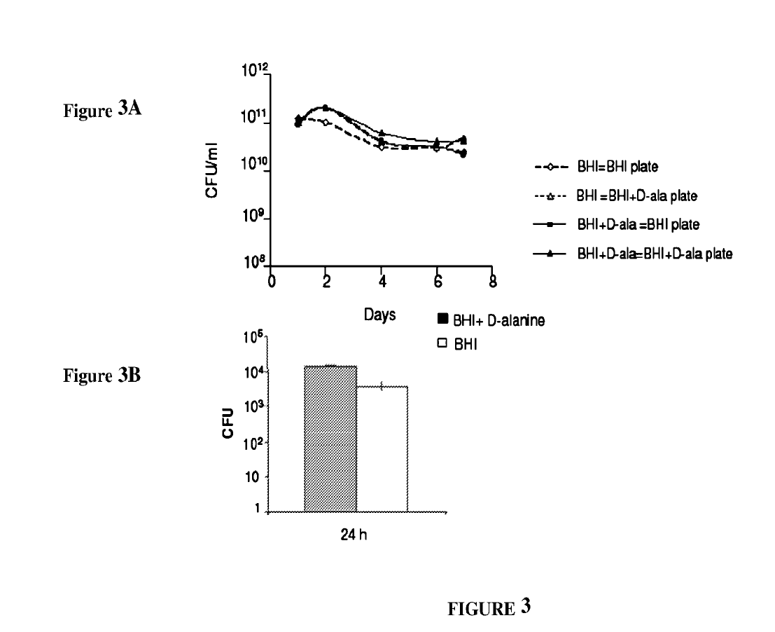

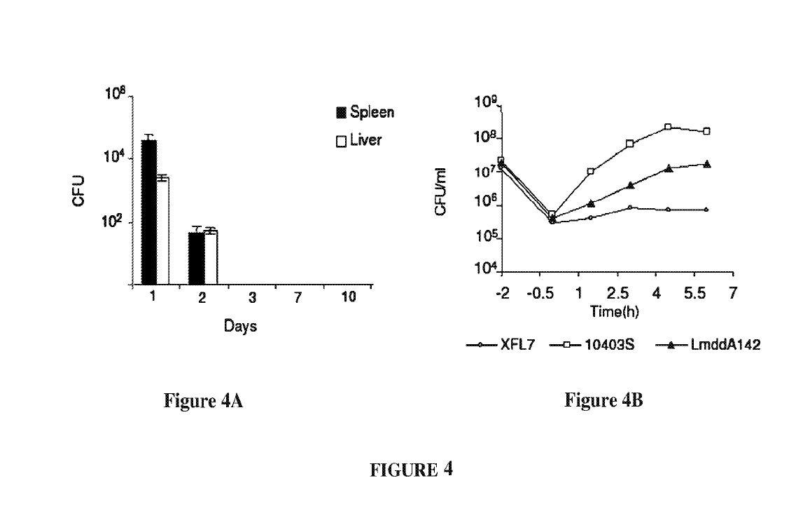

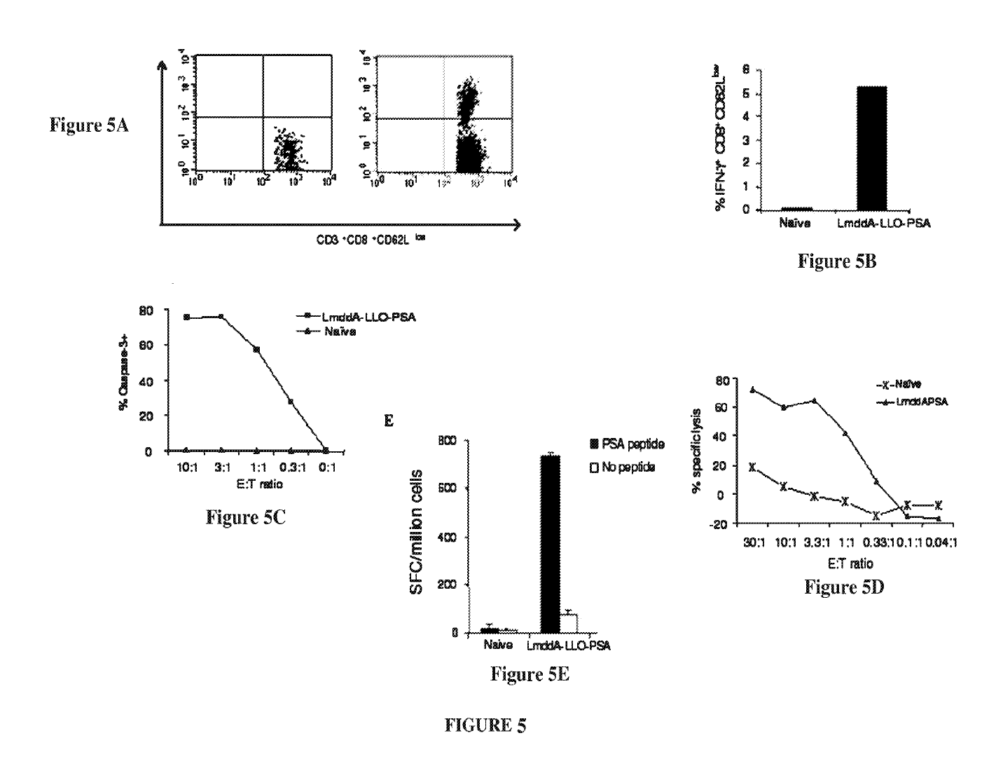

The in vitro stability of the plasmid was examined by culturing the LmddA-LLO-PSA Listeria strain in the presence or absence of selective pressure for eight days. The selective pressure for the strain LmddA-LLO-PSA is D-alanine. Therefore, the strain LmddA-LLO-PSA was passaged in Brain-Heart Infusion (BHI) and BHI₊ 100 μg/ml D-alanine. CFUs were determined for each day after plating on selective (BHI) and non-selective (BHI₊D-alanine) medium. It was expected that a loss of plasmid will result in higher CFU after plating on non-selective medium (BHI₊D-alanine). As depicted in FIG. 3A, there was no difference between the number of CFU in selective and non-selective medium. This suggests that the plasmid pAdv142 was stable for at least 50 generations, when the experiment was terminated.

Plasmid maintenance in vivo was determined by intravenous injection of 5×10⁷ CFU LmddA-LLO-PSA, in C57BL/6 mice. Viable bacteria were isolated from spleens homogenized in PBS at 24 h and 48 h. CFUs for each sample were determined at each time point on BHI plates and BHI₊100 mg/ml D-alanine. After plating the splenocytes on selective and non-selective medium, the colonies were recovered after 24 h. Since this strain is highly attenuated, the bacterial load is cleared in vivo in 24 h. No significant differences of CFUs were detected on selective and non-selective plates, indicating the stable presence of the recombinant plasmid in all isolated bacteria (FIG. 3B).

### Example 4: In Vivo Passaging, Virulence and Clearance of the Strain LmddA-142 (LmddA-LLO-PSA)

LmddA-142 is a recombinant Listeria strain that secretes the episomally expressed tLLO-PSA fusion protein. To determine a safe dose, mice were immunized with LmddA-LLO-PSA at various doses and toxic effects were determined. LmddA-LLO-PSA caused minimum toxic effects (data not shown). The results suggested that a dose of 108 CFU of LmddA-LLO-PSA was well tolerated by mice. Virulence studies indicate that the strain LmddA-LLO-PSA was highly attenuated.

The in vivo clearance of LmddA-LLO-PSA after administration of the safe dose, 10⁸ CFU intraperitoneally in C57BL/6 mice, was determined. There were no detectable colonies in the liver and spleen of mice immunized with LmddA-LLO-PSA after day 2. Since this strain is highly attenuated, it was completely cleared in vivo at 48 h (FIG. 4A).

To determine if the attenuation of LmddA-LLO-PSA attenuated the ability of the strain LmddA-LLO-PSA to infect macrophages and grow intracellularly, a cell infection assay was performed. Mouse macrophage-like cell line such as J774A.1, were infected in vitro with Listeria constructs and intracellular growth was quantified. The positive control strain, wild type Listeria strain 10403S grows intracellularly, and the negative control XFL7, a prfA mutant, cannot escape the phagolysosome and thus does not grow in J774 cells. The intracytoplasmic growth of LmddA-LLO-PSA was slower than 10403S due to the loss of the ability of this strain to spread from cell to cell (FIG. 4B). The results indicate that LmddA-LLO-PSA has the ability to infect macrophages and grow intracytoplasmically.

### Example 5: Immunogenicity of the Strain-LmddA-LLO-PSA in C57BL/6 Mice

The PSA-specific immune responses elicited by the construct LmddA-LLO-PSA in C57BL/6 mice were determined using PSA tetramer staining. Mice were immunized twice with LmddA-LLO-PSA at one week intervals and the splenocytes were stained for PSA tetramer on day 6 after the boost. Staining of splenocytes with the PSA-specific tetramer showed that LmddA-LLO-PSA elicited 23% of PSA tetramer⁺CD8⁺CD62 L^{low} cells (FIG. 5A). The functional ability of the PSA-specific T cells to secrete IFN-γ after stimulation with PSA peptide for 5 h was examined using intracellular cytokine staining. There was a 200-fold increase in the percentage of CD8⁺CD62 L^{low}IFN-γ secreting cells stimulated with PSA peptide in the LmddA-LLO-PSA group compared to the naïve mice (FIG. 5B), indicating that the LmddA-LLO-PSA strain is very immunogenic and primes high levels of functionally active PSA CD8⁺ T cell responses against PSA in the spleen.

To determine the functional activity of cytotoxic T cells generated against PSA after immunizing mice with LmddA-LLO-PSA, we tested the ability of PSA-specific CTLs to lyse cells EL4 cells pulsed with H-2D^{b} peptide in an in vitro assay. A FACS-based caspase assay (FIG. 5C) and Europium release (FIG. 5D) were used to measure cell lysis. Splenocytes of mice immunized with LmddA-LLO-PSA contained CTLs with high cytolytic activity for the cells that display PSA peptide as a target antigen.

Elispot was performed to determine the functional ability of effector T cells to secrete IFN-γ after 24 h stimulation with antigen. Using ELISpot, a 20-fold increase in the number of spots for IFN-γ in splenocytes from mice immunized with LmddA-LLO-PSA stimulated with specific peptide when compared to the splenocytes of the naïve mice was observed (FIG. 5E).

### Example 6: Immunization with the LmddA-142 Strains Induces Regression of a Tumor Expressing PSA and Infiltration of the Tumor by PSA-Specific CTLs

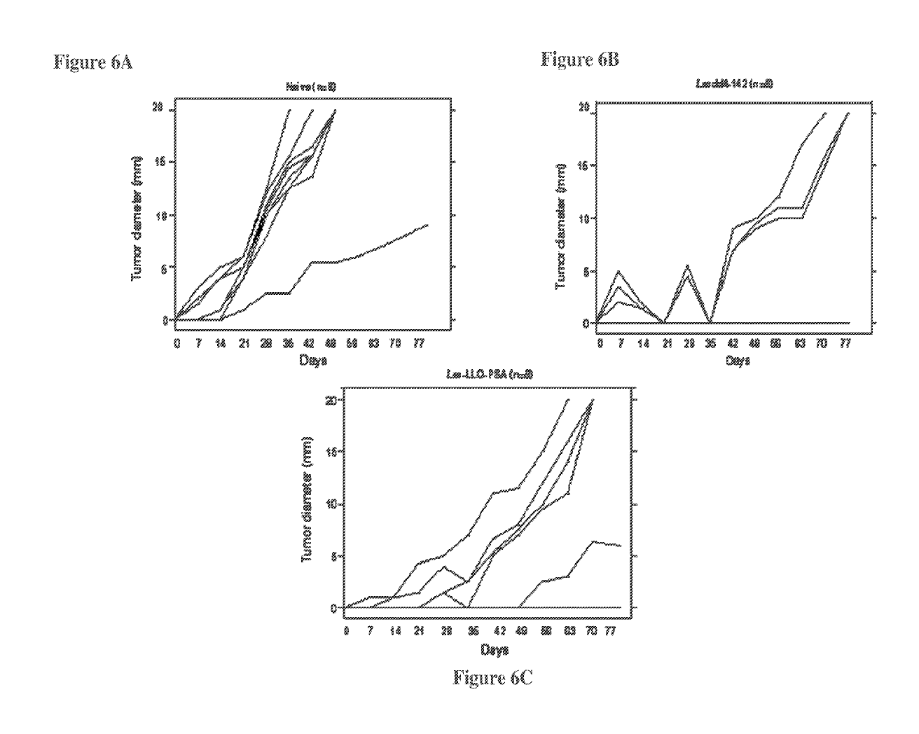

The therapeutic efficacy of the construct LmddA-142 (LmddA-LLO-PSA) was determined using a prostrate adenocarcinoma cell line engineered to express PSA (Tramp-C1-PSA (TPSA); Shahabi et al., 2008). Mice were subcutaneously implanted with 2×10⁶ TPSA cells. When tumors reached the palpable size of 4-6 mm, on day 6 after tumor inoculation, mice were immunized three times at one week intervals with 10⁸ CFU LmddA-142, 10⁷ CFU Lm-LLO-PSA (positive control) or left untreated. The naïve mice developed tumors gradually (FIG. 6A). The mice immunized with LmddA-142 were all tumor-free until day 35 and gradually 3 out of 8 mice developed tumors, which grew at a much slower rate as compared to the naïve mice (FIG. 6B). Five out of eight mice remained tumor free through day 70. As expected, Lm-LLO-PSA-vaccinated mice had fewer tumors than naïve controls and tumors developed more slowly than in controls (FIG. 6C). Thus, the construct LmddA-LLO-PSA could regress 60% of the tumors established by TPSA cell line and slow the growth of tumors in other mice. Cured mice that remained tumor free were rechallenged with TPSA tumors on day 68.

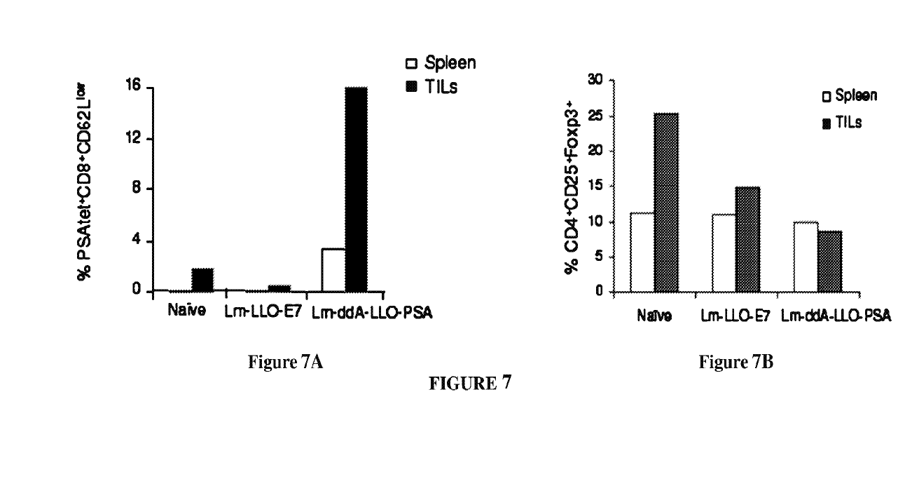

Immunization of mice with the LmddA-142 can control the growth and induce regression of 7-day established Tramp-C1 tumors that were engineered to express PSA in more than 60% of the experimental animals (FIG. 7B), compared to none in the untreated group (FIG. 7A). The LmddA-142 was constructed using a highly attenuated vector (LmddA) and the plasmid pADV142 (Table 1).

Further, the ability of PSA-specific CD8 lymphocytes generated by the LmddA-LLO-PSA construct to infiltrate tumors was investigated. Mice were subcutaneously implanted with a mixture of tumors and matrigel followed by two immunizations at seven day intervals with naïve or control (Lm-LLO-E7) Listeria, or with LmddA-LLO-PSA. Tumors were excised on day 21 and were analyzed for the population of CD8⁺CD62 L^{low}PSA^{tetramer+} and CD4⁺ CD25⁺FoxP3⁺ regulatory T cells infiltrating in the tumors.

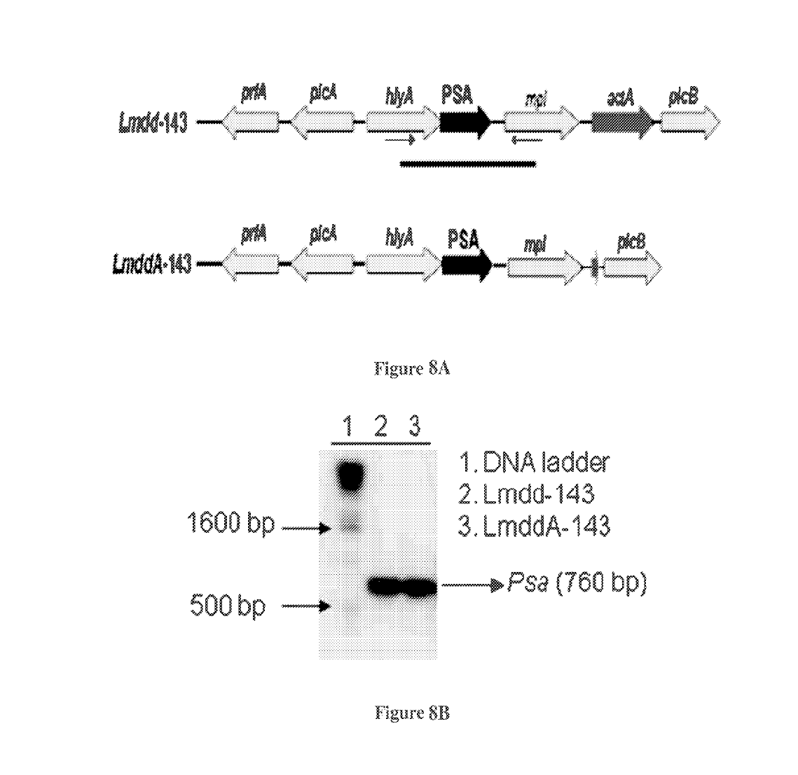

A very low number of CD8⁺CD62 L^{low} PSA^{tetramer+} tumor infiltrating lymphocytes (TILs) specific for PSA that were present in the both naïve and Lm-LLO-E7 control immunized mice was observed. However, there was a 10-30-fold increase in the percentage of PSA-specific CD8⁺CD62 L^{low} PSA^{tetramer+} TILs in the mice immunized with LmddA-LLO-PSA (FIG. 7A). Interestingly, the population of CD8+CD62 L^{low} PSA^{tetramer+} cells in spleen was 7.5 fold less than in tumor (FIG. 8A).

In addition, the presence of CD4⁺/CD25⁺/Foxp3⁺ T regulatory cells (Tregs) in the tumors of untreated mice and Listeria immunized mice was determined. Interestingly, immunization with Listeria resulted in a considerable decrease in the number of CD4⁺CD25⁺FoxP3⁺ T-regs in tumor but not in spleen (FIG. 8B). However, the construct LmddA-LLO-PSA had a stronger impact in decreasing the frequency of CD4⁺ CD25⁺FoxP3⁺ T-regs in tumors when compared to the naïve and Lm-LLO-E7 immunized group (FIG. 8B).

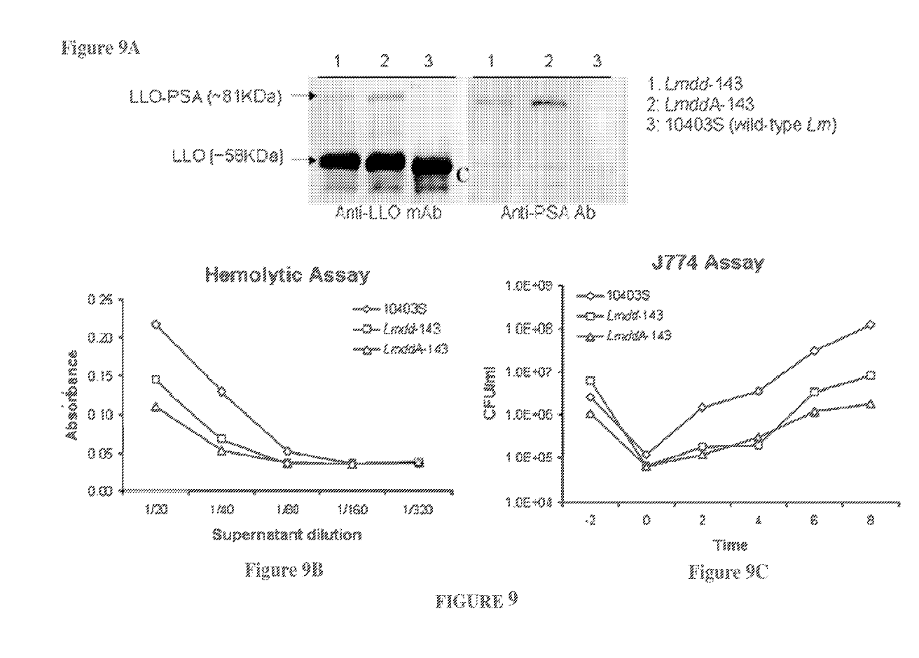

Thus, the LmddA-142 immunotherapy can induce PSA-specific CD8⁺ T cells that are able to infiltrate the tumor site (FIG. 9A). Interestingly, immunization with LmddA-142 was associated with a decreased number of regulatory T cells in the tumor (FIG. 9B), probably creating a more favorable environment for an efficient anti-tumor CTL activity.

### Example 7: Lmdd-143 and LmddA-143 Secretes a Functional LLO Despite the PSA Fusion

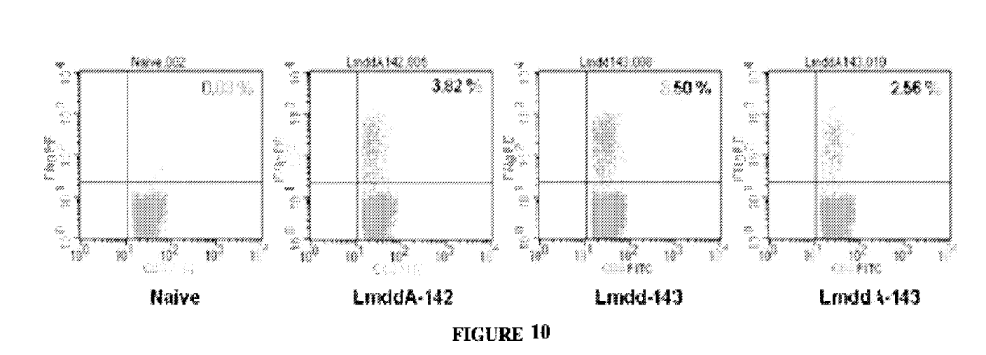

The Lmdd-143 and LmddA-143 contain the full-length human klk3 gene, which encodes the PSA protein, inserted by homologous recombination downstream and in frame with the hly gene in the chromosome. These constructs were made by homologous recombination using the pKSV7 plasmid (Smith and Youngman, Biochimie. 1992; 74 (7-8) p705-711), which has a temperature-sensitive replicon, carrying the hly-klk3-mpl recombination cassette. Because of the plasmid excision after the second recombination event, the antibiotic resistance marker used for integration selection is lost. Additionally, the actA gene is deleted in the LmddA-143 strain (FIG. 10A). The insertion of klk3 in frame with hly into the chromosome was verified by PCR (FIG. 10B) and sequencing (data not shown) in both constructs.

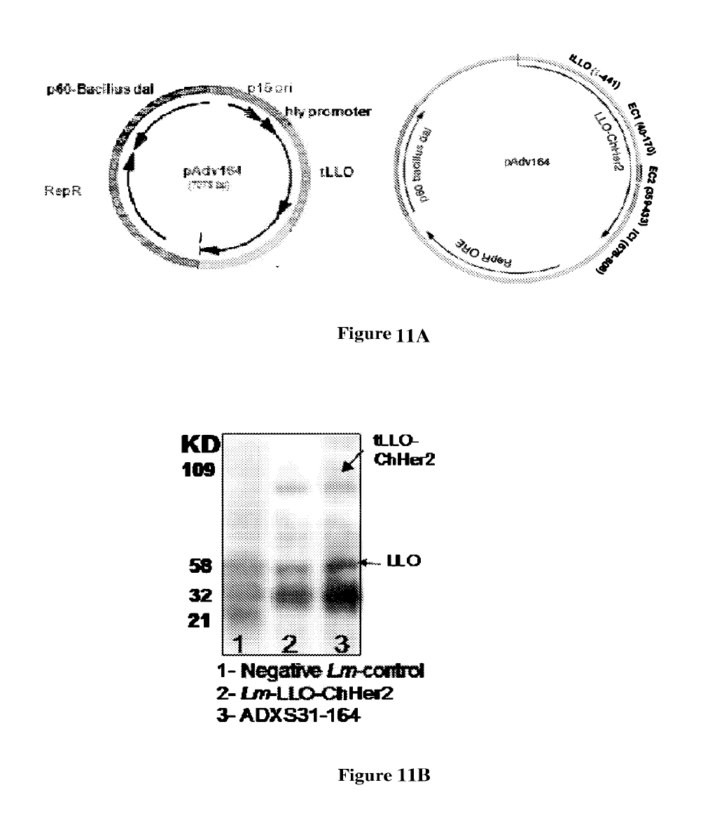

One important aspect of these chromosomal constructs is that the production of LLO-PSA would not completely abolish the function of LLO, which is required for escape of Listeria from the phagosome, cytosol invasion and efficient immunity generated by L. monocytogenes. Western-blot analysis of secreted proteins from Lmdd-143 and LmddA-143 culture supernatants revealed an ˜81 kDa band corresponding to the LLO-PSA fusion protein and an ˜60 kDa band, which is the expected size of LLO (FIG. 11A), indicating that LLO is either cleaved from the LLO-PSA fusion or still produced as a single protein by L. monocytogenes, despite the fusion gene in the chromosome. The LLO secreted by Lmdd-143 and LmddA-143 retained 50% of the hemolytic activity, as compared to the wild-type L. monocytogenes 10403S (FIG. 11B). In agreement with these results, both Lmdd-143 and LmddA-143 were able to replicate intracellularly in the macrophage-like J774 cell line (FIG. 11C).

### Example 8: Both Lmdd-143 and LmddA-143 Elicit Cell-Mediated Immune Responses Against the PSA Antigen

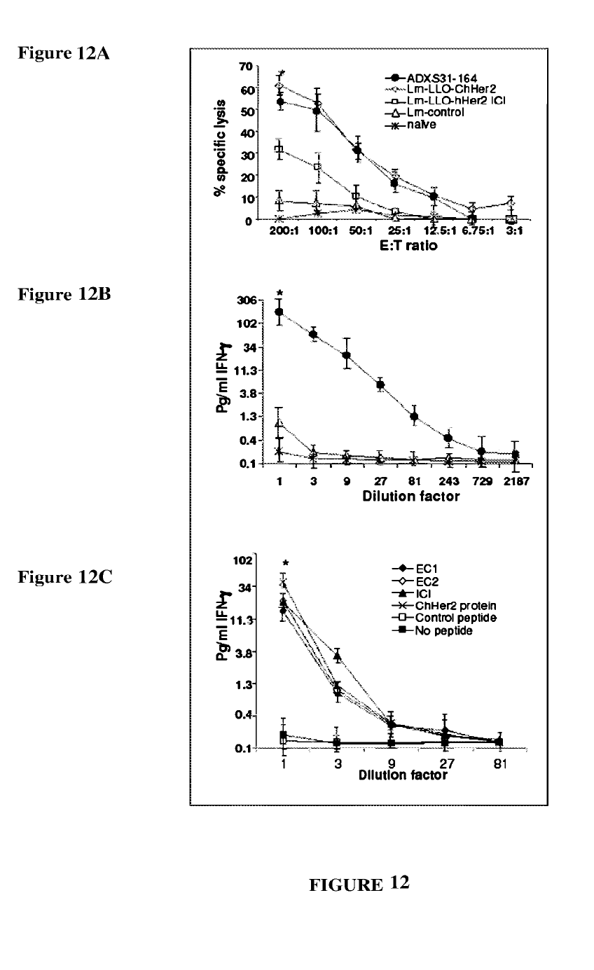

After showing that both Lmdd-143 and LmddA-143 were able to secrete PSA fused to LLO, the question of if these strains could elicit PSA-specific immune responses in vivo was investigated. C57Bl/6 mice were either left untreated or immunized twice with the Lmdd-143, LmddA-143 or LmddA-142. PSA-specific CD8+ T cell responses were measured by stimulating splenocytes with the PSA65-74 peptide and intracellular staining for IFN-γ. As shown in FIG. 12, the immune response induced by the chromosomal and the plasmid-based vectors is similar.

### Example 9: Generation of L. Monocytogenes Strains that Secrete LLO Fragments Fused to her-2 Fragments: Construction of ADXS31-164

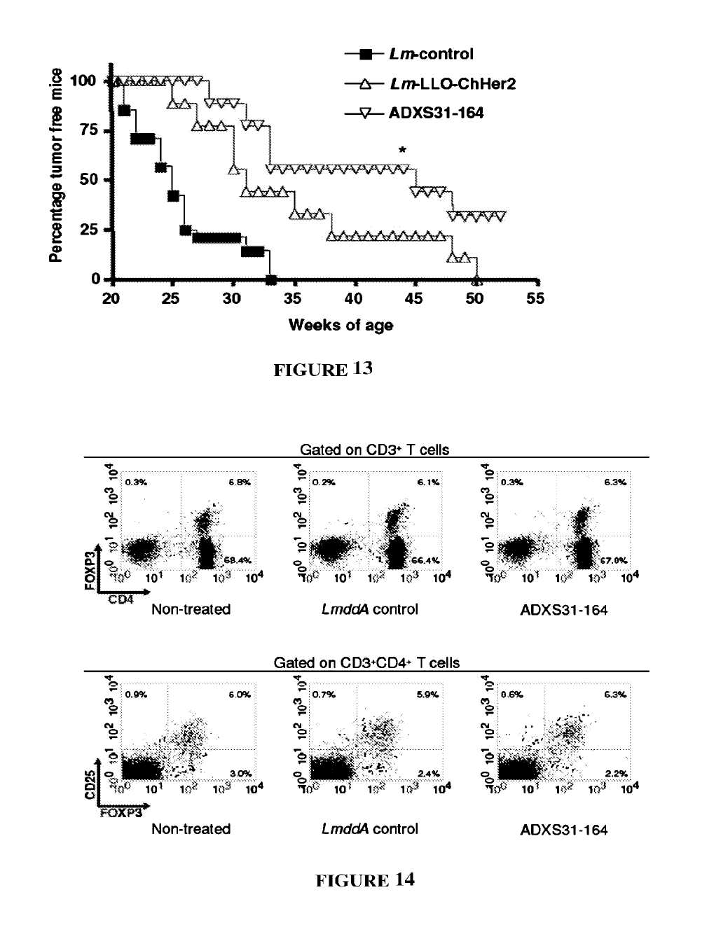

Construction of the chimeric Her2/neu gene (ChHer2) was as follows. Briefly, ChHer2 gene was generated by direct fusion of two extracellular (aa 40-170 and aa 359-433) and one intracellular fragment (aa 678-808) of the Her2/neu protein by SOEing PCR method. The chimeric protein harbors most of the known human MHC class I epitopes of the protein. ChHer2 gene was excised from the plasmid, pAdv138 (which was used to construct Lm-LLO-ChHer2) and cloned into LmddA shuttle plasmid, resulting in the plasmid pAdv164 (FIG. 13A). There are two major differences between these two plasmid backbones. 1) Whereas pAdv138 uses the chloramphenicol resistance marker (cat) for in vitro selection of recombinant bacteria, pAdv164 harbors the D-alanine racemase gene (dal) from bacillus subtilis, which uses a metabolic complementation pathway for in vitro selection and in vivo plasmid retention in LmddA strain which lacks the dal-dat genes. This immunotherapy platform was designed and developed to address FDA concerns about the antibiotic resistance of the engineered Listeria immunotherapy strains. 2) Unlike pAdv138, pAdv164 does not harbor a copy of the prfA gene in the plasmid (see sequence below and FIG. 13A), as this is not necessary for in vivo complementation of the Lmdd strain. The LmddA immunotherapy strain also lacks the actA gene (responsible for the intracellular movement and cell-to-cell spread of Listeria) so the recombinant immunotherapy strains derived from this backbone are 100 times less virulent than those derived from the Lmdd, its parent strain. LmddA-based immunotherapies are also cleared much faster (in less than 48 hours) than the Lmdd-based immunotherapies from the spleens of the immunized mice. The expression and secretion of the fusion protein tLLO-ChHer2 from this strain was comparable to that of the Lm-LLO-ChHer2 in TCA precipitated cell culture supernatants after 8 hours of in vitro growth (FIG. 13B) as a band of ˜104 KD was detected by an anti-LLO antibody using Western Blot analysis. The Listeria backbone strain expressing only tLLO was used as negative control.

pAdv164 sequence (7075 base pairs) (see FIGS. 13A and 13B):
(SEQ ID NO: 35)
cggagtgtatactggcttactatgttggcactgatgagggtgtcagtgaagtgcttcatgtggcaggagaaaaaaggct
gcaccggtgcgtcagcagaatatgtgatacaggatatattccgcttcctcgctcactgactcgctacgctcggtcgttcgactgcggcg
agcggaaatggcttacgaacggggcggagatttcctggaagatgccaggaagatacttaacagggaagtgagagggccgcggca
aagccgtttttccataggctccgcccccctgacaagcatcacgaaatctgacgctcaaatcagtggtggcgaaacccgacaggactat
aaagataccaggcgtttccccctggcggctccctcgtgcgctctcctgttcctgcctttcggtttaccggtgtcattccgctgttatggccg
cgtttgtctcattccacgcctgacactcagttccgggtaggcagttcgctccaagctggactgtatgcacgaaccccccgttcagtccga
ccgctgcgccttatccggtaactatcgtcttgagtccaacccggaaagacatgcaaaagcaccactggcagcagccactggtaattga
tttagaggagttagtcttgaagtcatgcgccggttaaggctaaactgaaaggacaagttttggtgactgcgctcctccaagccagttacc
tcggttcaaagagttggtagctcagagaaccttcgaaaaaccgccctgcaaggcggttttttcgttttcagagcaagagattacgcgca
gaccaaaacgatctcaagaagatcatcttattaatcagataaaatatttctagccctcctttgattagtatattcctatcttaaagttacttttat
gtggaggcattaacatttgttaatgacgtcaaaaggatagcaagactagaataaagctataaagcaagcatataatattgcgtttcatcttt
agaagcgaatttcgccaatattataattatcaaaagagaggggtggcaaacggtatttggcattattaggttaaaaaatgtagaaggaga
gtgaaacccatgaaaaaaataatgctagtttttattacacttatattagttagtctaccaattgcgcaacaaactgaagcaaaggatgcatc
tgcattcaataaagaaaattcaatttcatccatggcaccaccagcatctccgcctgcaagtcctaagacgccaatcgaaaagaaacacg
cggatgaaatcgataagtatatacaaggattggattacaataaaaacaatgtattagtataccacggagatgcagtgacaaatgtgccg
ccaagaaaaggttacaaagatggaaatgaatatattgttgtggagaaaaagaagaaatccatcaatcaaaataatgcagacattcaagt
tgtgaatgcaatttcgagcctaacctatccaggtgctctcgtaaaagcgaattcggaattagtagaaaatcaaccagatgttctccctgta
aaacgtgattcattaacactcagcattgatttgccaggtatgactaatcaagacaataaaatagttgtaaaaaatgccactaaatcaaacg
ttaacaacgcagtaaatacattagtggaaagatggaatgaaaaatatgctcaagcttatccaaatgtaagtgcaaaaattgattatgatga
cgaaatggcttacagtgaatcacaattaattgcgaaatttggtacagcatttaaagctgtaaataatagcttgaatgtaaacttcggcgca
atcagtgaagggaaaatgcaagaagaagtcattagttttaaacaaatttactataacgtgaatgttaatgaacctacaagaccttccagat
ttttcggcaaagctgttactaaagagcagttgcaagcgcttggagtgaatgcagaaaatcctcctgcatatatctcaagtgtggcgtatg
gccgtcaagtttatttgaaattatcaactaattcccatagtactaaagtaaaagctgcttttgatgctgccgtaagcggaaaatctgtctcag
gtgatgtagaactaacaaatatcatcaaaaattcttccttcaaagccgtaatttacggaggttccgcaaaagatgaagttcaaatcatcga
cggcaacctcggagacttacgcgatattttgaaaaaaggcgctacttttaatcgagaaacaccaggagttcccattgcttatacaacaaa
cttcctaaaagacaatgaattagctgttattaaaaacaactcagaatatattgaaacaacttcaaaagcttatacagatggaaaaattaaca
tcgatcactctggaggatacgttgctcaattcaacatttcttgggatgaagtaaattatgatctcgagacccacctggacatgctccgcca
cctctaccagggctgccaggtggtgcagggaaacctggaactcacctacctgcccaccaatgccagcctgtccttcctgcaggatatc
caggaggtgcagggctacgtgctcatcgctcacaaccaagtgaggcaggtcccactgcagaggctgcggattgtgcgaggcaccc
agctctttgaggacaactatgccctggccgtgctagacaatggagacccgctgaacaataccacccctgtcacaggggcctccccag
gaggcctgcgggagctgcagcttcgaagcctcacagagatcttgaaaggaggggtcttgatccagcggaacccccagctctgctac
caggacacgattttgtggaagaatatccaggagtttgctggctgcaagaagatctttgggagcctggcatttctgccggagagctttgat
ggggacccagcctccaacactgccccgctccagccagagcagctccaagtgtttgagactctggaagagatcacaggttacctatac
atctcagcatggccggacagcctgcctgacctcagcgtcttccagaacctgcaagtaatccggggacgaattctgcacaatggcgcct
actcgctgaccctgcaagggctgggcatcagctggctggggctgcgctcactgagggaactgggcagtggactggccctcatccac
cataacacccacctctgcttcgtgcacacggtgccctgggaccagctctttcggaacccgcaccaagctctgctccacactgccaacc
ggccagaggacgagtgtgtgggcgagggcctggcctgccaccagctgtgcgcccgagggcagcagaagatccggaagtacacg
atgcggagactgctgcaggaaacggagctggtggagccgctgacacctagcggagcgatgcccaaccaggcgcagatgcggatc
ctgaaagagacggagctgaggaaggtgaaggtgcttggatctggcgcttttggcacagtctacaagggcatctggatccctgatggg
gagaatgtgaaaattccagtggccatcaaagtgttgagggaaaacacatcccccaaagccaacaaagaaatcttagacgaagcatac
gtgatggctggtgtgggctccccatatgtctcccgccttctgggcatctgcctgacatccacggtgcagctggtgacacagcttatgcc
ctatggctgcctcttagactaatctagacccgggccactaactcaacgctagtagtggatttaatcccaaatgagccaacagaaccaga
accagaaacagaacaagtaacattggagttagaaatggaagaagaaaaaagcaatgatttcgtgtgaataatgcacgaaatcattgctt
atttttttaaaaagcgatatactagatataacgaaacaacgaactgaataaagaatacaaaaaaagagccacgaccagttaaagcctga
gaaactttaactgcgagccttaattgattaccaccaatcaattaaagaagtcgagacccaaaatttggtaaagtatttaattactttattaatc
agatacttaaatatctgtaaacccattatatcgggtttttgaggggatttcaagtctttaagaagataccaggcaatcaattaagaaaaactt
agttgattgccttttttgttgtgattcaactttgatcgtagcttctaactaattaattttcgtaagaaaggagaacagctgaatgaatatccctttt
gttgtagaaactgtgcttcatgacggcttgttaaagtacaaatttaaaaatagtaaaattcgctcaatcactaccaagccaggtaaaagta
aaggggctatttttgcgtatcgctcaaaaaaaagcatgattggcggacgtggcgttgttctgacttccgaagaagcgattcacgaaaatc
aagatacatttacgcattggacaccaaacgtttatcgttatggtacgtatgcagacgaaaaccgttcatacactaaaggacattctgaaaa
caatttaagacaaatcaataccttctttattgattttgatattcacacggaaaaagaaactatttcagcaagcgatattttaacaacagctatt
gatttaggttttatgcctacgttaattatcaaatctgataaaggttatcaagcatattttgttttagaaacgccagtctatgtgacttcaaaatca
gaatttaaatctgtcaaagcagccaaaataatctcgcaaaatatccgagaatattttggaaagtctttgccagttgatctaacgtgcaatca
ttttgggattgctcgtataccaagaacggacaatgtagaattttttgatcccaattaccgttattctttcaaagaatggcaagattggtctttca
aacaaacagataataagggctttactcgttcaagtctaacggttttaagcggtacagaaggcaaaaaacaagtagatgaaccctggttt
aatctcttattgcacgaaacgaaattttcaggagaaaagggtttagtagggcgcaatagcgttatgtttaccctctctttagcctactttagtt
caggctattcaatcgaaacgtgcgaatataatatgtttgagtttaataatcgattagatcaacccttagaagaaaaagaagtaatcaaaatt
gttagaagtgcctattcagaaaactatcaaggggctaatagggaatacattaccattctttgcaaagcttgggtatcaagtgatttaacca
gtaaagatttatttgtccgtcaagggtggtttaaattcaagaaaaaaagaagcgaacgtcaacgtgttcatttgtcagaatggaaagaag
atttaatggcttatattagcgaaaaaagcgatgtatacaagccttatttagcgacgaccaaaaaagagattagagaagtgctaggcattc
ctgaacggacattagataaattgctgaaggtactgaaggcgaatcaggaaattttctttaagattaaaccaggaagaaatggtggcattc
aacttgctagtgttaaatcattgttgctatcgatcattaaattaaaaaaagaagaacgagaaagctatataaaggcgctgacagcttcgttt
aatttagaacgtacatttattcaagaaactctaaacaaattggcagaacgccccaaaacggacccacaactcgatttgtttagctacgata
caggctgaaaataaaacccgcactatgccattacatttatatctatgatacgtgtttgatttctttgctggctagcttaattgcttatatttacct
gcaataaaggatttcttacttccattatactcccattttccaaaaacatacggggaacacgggaacttattgtacaggccacctcatagtta
atggtttcgagccttcctgcaatctcatccatggaaatatattcatccccctgccggcctattaatgtgacttttgtgcccggcggatattcc
tgatccagctccaccataaattggtccatgcaaattcggccggcaattttcaggcgttttcccttcacaaggatgtcggtccctttcaatttt
cggagccagccgtccgcatagcctacaggcaccgtcccgatccatgtgtctttttccgctgtgtactcggctccgtagctgacgctctc
gccttttctgatcagtttgacatgtgacagtgtcgaatgcagggtaaatgccggacgcagctgaaacggtatctcgtccgacatgtcag
cagacgggcgaaggccatacatgccgatgccgaatctgactgcattaaaaaagccttttttcagccggagtccagcggcgctgttcgc
gcagtggaccattagattctttaacggcagcggagcaatcagctctttaaagcgctcaaactgcattaagaaatagcctctttctttttcat
ccgctgtcgcaaaatgggtaaatacccctttgcactttaaacgagggttgcggtcaagaattgccatcacgttctgaacttcttcctctgttt
ttacaccaagtctgttcatccccgtatcgaccttcagatgaaaatgaagagaaccttttttcgtgtggcgggctgcctcctgaagccattc
aacagaataacctgttaaggtcacgtcatactcagcagcgattgccacatactccgggggaaccgcgccaagcaccaatataggcgc
cttcaatccctttttgcgcagtgaaatcgcttcatccaaaatggccacggccaagcatgaagcacctgcgtcaagagcagcctttgctgt
ttctgcatcaccatgcccgtaggcgtttgctttcacaactgccatcaagtggacatgttcaccgatatgattttcatattgctgacattttcctt
tatcgcggacaagtcaatttccgcccacgtatctctgtaaaaaggttttgtgctcatggaaaactcctctcttttttcagaaaatcccagtac
gtaattaagtatttgagaattaattttatattgattaatactaagtttacccagttttcacctaaaaaacaaatgatgagataatagctccaaag
gctaaagaggactataccaactatttgttaattaa

### Example 10: ADXS31-164 is as Immunogenic as Lm-LLO-ChHER2

Immunogenic properties of ADXS31-164 in generating anti-Her2/neu specific cytotoxic T cells were compared to those of the Lm-LLO-ChHer2 immunotherapy in a standard CTL assay. Both immunotherapies elicited strong but comparable cytotoxic T cell responses toward Her2/neu antigen expressed by 3T3/neu target cells. Accordingly, mice immunized with a Listeria expressing only an intracellular fragment of Her2-fused to LLO showed lower lytic activity than the chimeras which contain more MHC class I epitopes. No CTL activity was detected in naïve animals or mice injected with the irrelevant Listeria immunotherapy (FIG. 14A). ADXS31-164 was also able to stimulate the secretion of IFN-γ by the splenocytes from wild type FVB/N mice (FIG. 14B). This was detected in the culture supernatants of these cells that were co-cultured with mitomycin C treated NT-2 cells, which express high levels of Her2/neu antigen (FIG. 14C).

Proper processing and presentation of the human MHC class I epitopes after immunizations with ADXS31-164 was tested in HLA-A2 mice. Splenocytes from immunized HLA-A2 transgenics were co-incubated for 72 hours with peptides corresponding to mapped HLA-A2 restricted epitopes located at the extracellular (HLYQGCQVV SEQ ID NO: 36 or KIFGSLAFL SEQ ID NO: 37) or intracellular (RLLQETELV SEQ ID NO: 38) domains of the Her2/neu molecule (FIG. 14C). A recombinant ChHer2 protein was used as positive control and an irrelevant peptide or no peptide as negative controls. The data from this experiment show that ADXS31-164 is able to elicit anti-Her2/neu specific immune responses to human epitopes that are located at different domains of the targeted antigen.

### Example 11: ADXS31-164 was More Efficacious than Lm-LLO-ChHER2 in Preventing the Onset of Spontaneous Mammary Tumors

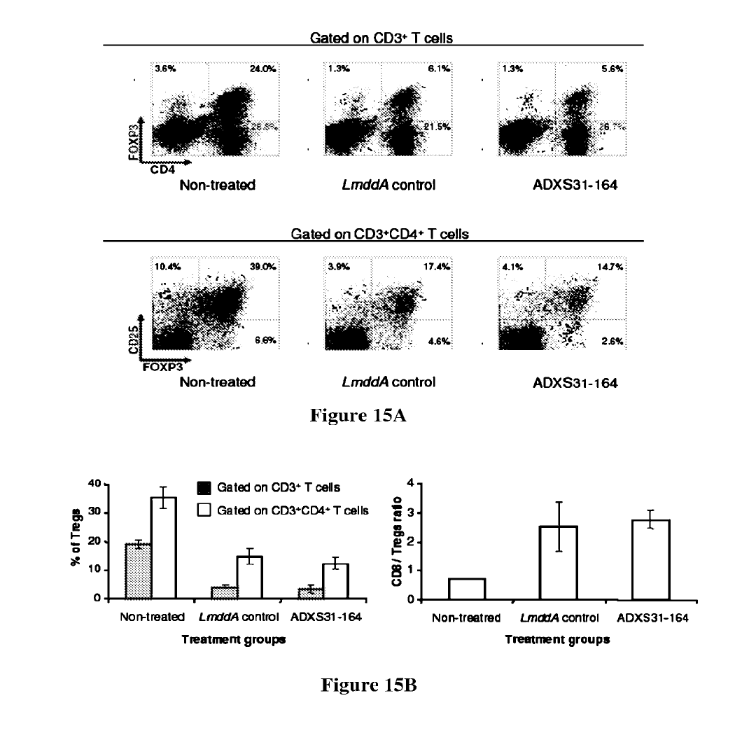

Anti-tumor effects of ADXS31-164 were compared to those of Lm-LLO-ChHer2 in Her2/neu transgenic animals which develop slow growing, spontaneous mammary tumors at 20-25 weeks of age. All animals immunized with the irrelevant Listeria-control immunotherapy developed breast tumors within weeks 21-25 and were sacrificed before week 33. In contrast, Listeria-Her2/neu recombinant immunotherapies caused a significant delay in the formation of the mammary tumors. On week 45, more than 50% of ADXS31-164 vaccinated mice (5 out of 9) were still tumor free, as compared to 25% of mice immunized with Lm-LLO-ChHer2. At week 52, 2 out of 8 mice immunized with ADXS31-164 still remained tumor free, whereas all mice from other experimental groups had already succumbed to their disease (FIG. 15). These results indicate that despite being more attenuated, ADXS31-164 is more efficacious than Lm-LLO-ChHer2 in preventing the onset of spontaneous mammary tumors in Her2/neu transgenic animals.

### Example 12: Mutations in HER2/Neu Gene Upon Immunization with ADXS31-164

Mutations in the MHC class I epitopes of Her2/neu have been considered responsible for tumor escape upon immunization with small fragment immunotherapies or trastuzumab (Herceptin), a monoclonal antibody that targets an epitope in the extracellular domain of Her2/neu. To assess this, genomic material was extracted from the escaped tumors in the transgenic animals and sequenced the corresponding fragments of the neu gene in tumors immunized with the chimeric or control immunotherapies. Mutations were not observed within the Her-2/neu gene of any vaccinated tumor samples suggesting alternative escape mechanisms (data not shown).

### Example 13: ADXS31-164 Causes a Significant Decrease in Intra-Tumoral T Regulatory Cells

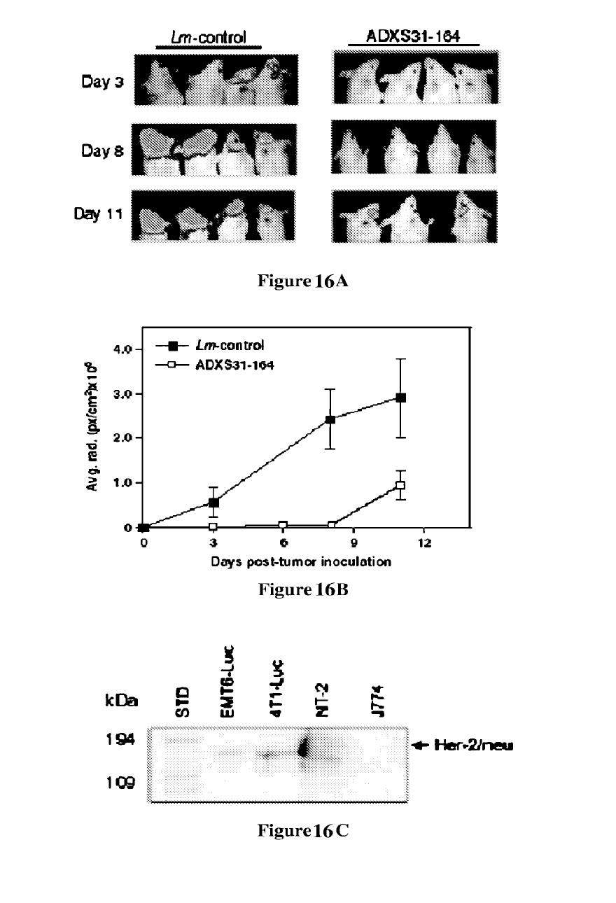

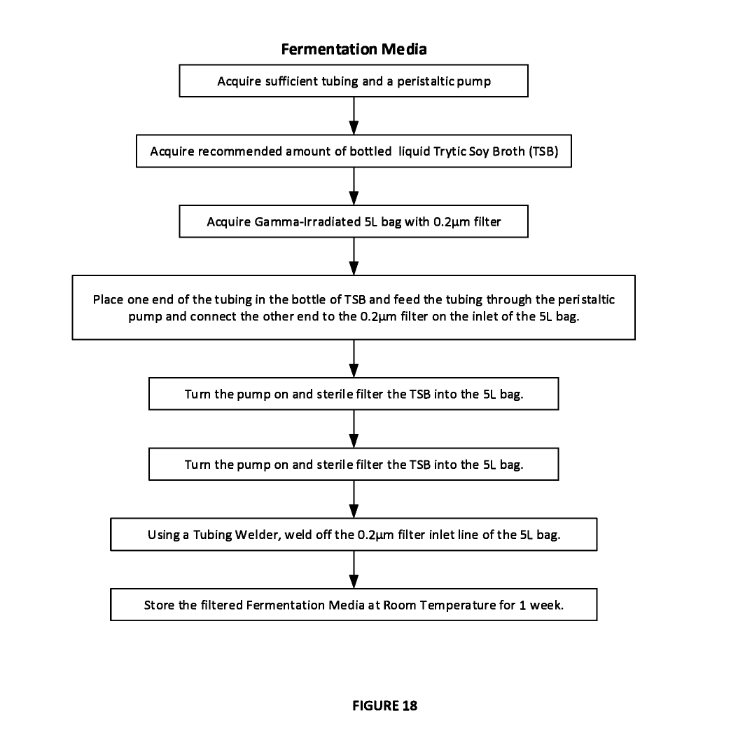

To elucidate the effect of ADXS31-164 on the frequency of regulatory T cells in spleens and tumors, mice were implanted with NT-2 tumor cells. Splenocytes and intra-tumoral lymphocytes were isolated after three immunizations and stained for Tregs, which were defined as CD3⁺/CD4⁺/CD25⁺/FoxP3⁺ cells, although comparable results were obtained with either FoxP3 or CD25 markers when analyzed separately. The results indicated that immunization with ADXS31-164 had no effect on the frequency of Tregs in the spleens, as compared to an irrelevant Listeria immunotherapy or the naïve animals (FIG. 16). In contrast, immunization with the Listeria immunotherapies caused a considerable impact on the presence of Tregs in the tumors (FIG. 17A). Whereas in average 19.0% of all CD3⁺ T cells in untreated tumors were Tregs, this frequency was reduced to 4.2% for the irrelevant immunotherapy and 3.4% for ADXS31-164, a 5-fold reduction in the frequency of intra-tumoral Tregs (FIG. 18B). The decrease in the frequency of intra-tumoral Tregs in mice treated with either of the LmddA immunotherapies could not be attributed to differences in the sizes of the tumors. In a representative experiment, the tumors from mice immunized with ADXS31-164 were significantly smaller [mean diameter (mm)±SD, 6.71±0.43, n=5] than the tumors from untreated mice (8.69±0.98, n=5, p<0.01) or treated with the irrelevant immunotherapy (8.41±1.47, n=5, p=0.04), whereas comparison of these last two groups showed no statistically significant difference in tumor size (p=0.73). The lower frequency of Tregs in tumors treated with LmddA immunotherapies resulted in an increased intratumoral CD8/Tregs ratio, suggesting that a more favorable tumor microenvironment can be obtained after immunization with LmddA immunotherapies. However, only the immunotherapy expressing the target antigen HER2/neu (ADXS31-164) was able to reduce tumor growth, indicating that the decrease in Tregs has an effect only in the presence on antigen-specific responses in the tumor.

### Example 14: Peripheral Immunization with ADXS31-164 can Delay the Growth of a Metastatic Breast Cancer Cell Line in the Brain

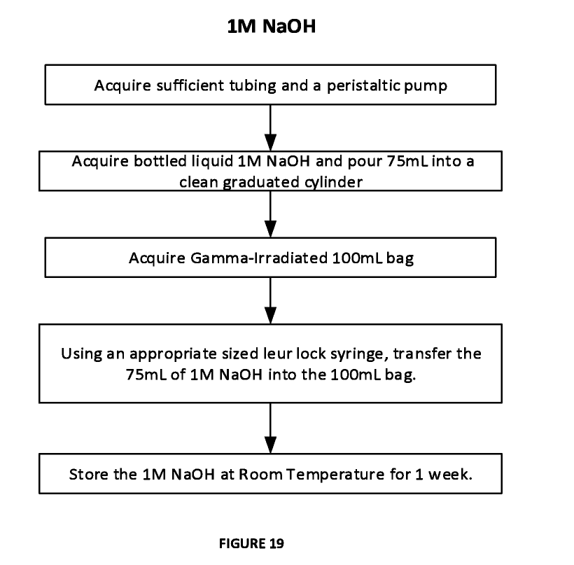

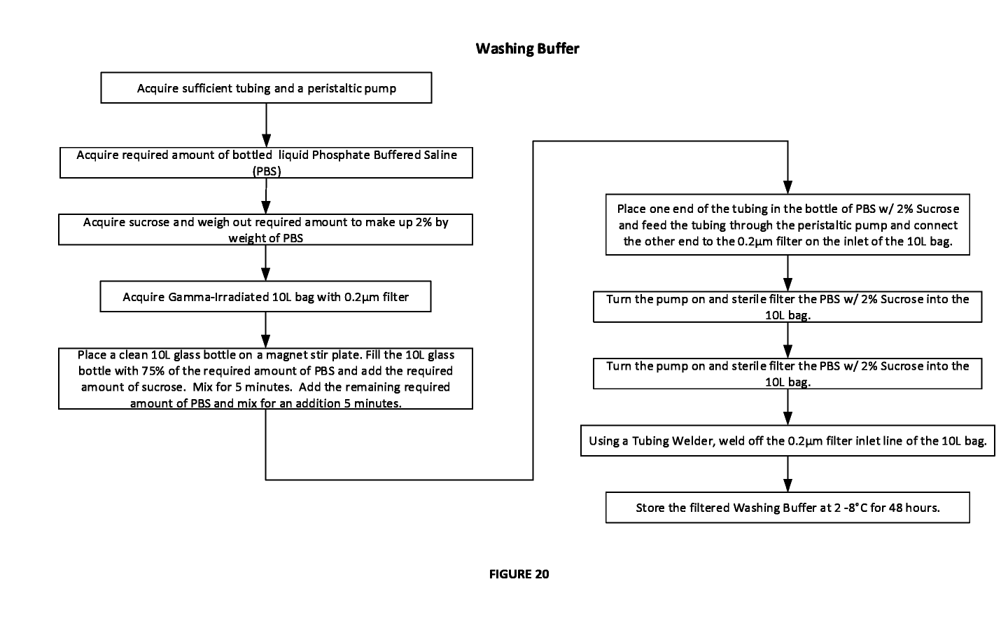

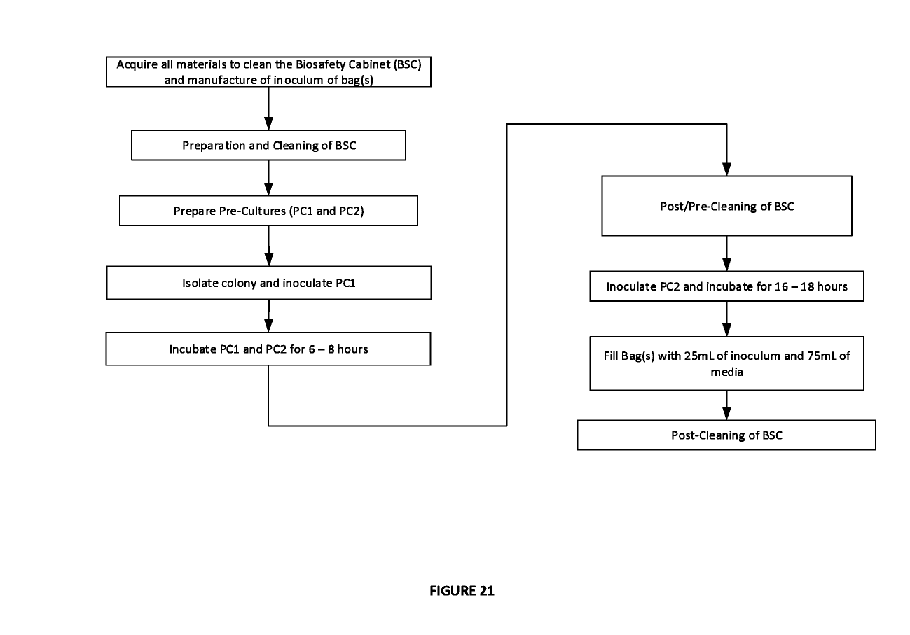

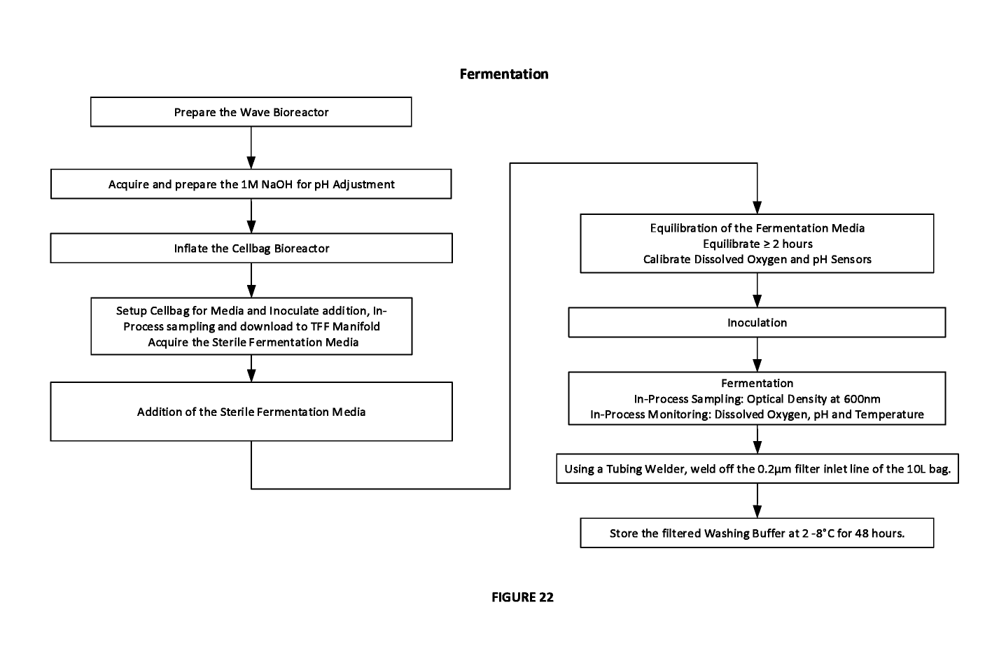

Mice were immunized IP with ADXS31-164 or irrelevant Lm-control immunotherapies and then implanted intra-cranially with 5,000 EMT6-Luc tumor cells, expressing luciferase and low levels of Her2/neu (FIG. 19A). Tumors were monitored at different times post-inoculation by ex vivo imaging of anesthetized mice. On day 8 post-tumor inoculation tumors were detected in all control animals, but none of the mice in ADXS31-164 group showed any detectable tumors (FIGS. 19A and 19B). ADXS31-164 could clearly delay the onset of these tumors, as on day 11 post-tumor inoculation all mice in negative control group had already succumbed to their tumors, but all mice in ADXS31-164 group were still alive and only showed small signs of tumor growth. These results strongly suggest that the immune responses obtained with the peripheral administration of ADXS31-164 could possibly reach the central nervous system and that LmddA-based immunotherapies might have a potential use for treatment of CNS tumors.

### Example 15: ADXS31-142 and ADXS31-164 Manufacturing Process

### Process Overview

The ADXS31-142/ADXS31-164 drug substance manufacturing process (FIG. 17) consists of four major steps:

A. Media Preparation

B. Pre-culture Process

C. Fermentation and Crossflow Process

D. Harvest and Aliquotation of drug substance

E. Aseptic Fill into Vials

### I. Process Description

A. Media Preparation

All media preparations are performed in a Grade C cleanroom, while all aseptic working steps are performed in a Grade A/B cleanroom. Non-sterile materials to be in contact with the media must be washed with water for injection (WFI) and autoclaved before the start of media preparation. Sterilized solutions have a specified expiration date of 8 weeks after preparation (see Table 3).

TABLE 3
Media Preparation
Formulation Table

| | | Component | | Release | | Storage | Expiry |
|---|---|---|---|---|---|---|---|
| Formulation | Components | Weights (g/L) | | Criteria | Specification | Conditions | Date |
| Flushing | Tryptic Soy | 40-70 | g | NA | NA | Room | 4-8 |
| Medium | Broth (TSB) | | | | | Temperature | weeks |
| (CFF) | granulate | | | | | | |
| | WFI | QS to 1-5 | L | | | | |
| Pre-culture | TSB granulate | 70-100 | g | NA | NA | Room | 4-8 |
| Medium | WFI | QS to 1-5 | L | | | Temperature | weeks |
| Fermentation | TSB granulate | 500-1000 | g | NA | NA | Room | 4-8 |
| Medium | Glucose | 100-300 | g | | | Temperature | weeks |
| | Yeast extract | 50-250 | g | | | | |
| | WFI | QS to 5-20 | L | | | | |
| pH Control | NaOH | 1-5 | L | NA | NA | Room | 4-8 |
| Solution | | | | | | Temperature | weeks |

a. Fermentation Medium

The main medium for fermentation is prepared directly before the start of production, and the transfer is performed via sterile filtration. The medium is based on tryptic soy broth (TSB). Preparation of the medium takes place in a 50 L mixtainer bag the mass of ingredients is calculated for a fermentation volume of 5-30 L due to the addition of 0.5-5 L pre-culture. First, 5-20 kg WFI are transferred into the bag. Afterwards, 500-1000 TSB granulate are added and solved completely with the integrated magnetic stirrer of the bag holder. Then, 100-300 g glucose and 50-250 g yeast extract are weighed in sterile containers and transferred into the bag (rinsed with 100±1 mg WFI). Total weight of the medium is 5-25 kg. Bioburden is tested by taking a 12±1 mL sample of the final solution in 15 mL sterile tubes. The mixtainer with the medium is closed and transferred into the pilot plant (Grade D cleanroom). For sterile filtration, the bag is connected with the autoclaved filtration line under a mobile laminar flow hood; filtration is already interlinked with the bag of the fermentor.

b. Pre-Culture Medium

Pre-culture medium is 1-5 L TSB. 70-100 g TSB granulate and 1-5 L WFI is mixed in a 5 L laboratory bottle and dissolved completely with a magnetic stir bar. To autoclave (121° C., 20 min) the 3 L is split into two 1.5 L aliquots in 2 L glass bottles, closed completely.

c. Base Solution

Base solution for pH control during fermentation is 1-5 L of NaOH. To prepare this, 1-5 L WFI are filled into a 5 L glass bottle, after which 50-450 g NaOH pellets are added. Final volume is adjusted to—−5 L by a 1 L graduated cylinder. Mass is noted for mass balance of the process.

d. Flushing Medium

The flushing medium for flushing filter cassettes (Cross Flow Filtration (CFF)) after sterilization is 1-5 L of TSB. 40-70 g TSB granulate and 1000-5000 mL WFI are mixed in a 1-10 L glass bottle and dissolved completely with a magnetic stir bar. This is then autoclaved (121° C., 20 min), with the glass bottle fitting up with a 2 ported bottle top, screw cap, venting filter silicone tubing, and sterile connector.

e. Washing Buffer

Approx. 5-20 L WFI is filled into a glass bottle. Other ingredients as specified in the Table 4 below are weighed, each in a sterile container, flushed and dissolved completely using a magnetic stirrer before the next raw material is added. The final solution is transferred into a sterile biotainer by measuring the complete volume in 1 L steps with a graduated cylinder. Adjustment up to 5-20 L is made with WFI. pH is then measured from a 3 mL sample taken with a 5 mL sterile pipette into a 15 mL sterile tube. The pH must be within 7.4±1.0. Adjustment does not take place; if target is not achieved, preparation has to be discarded and repeated. Bioburden is then analyzed from a 12±2 mL sample taken with a 25 mL sterile pipette into a 15 mL sterile tube. A second sterile biotainer is placed in Grade A cleanroom, arranged with the autoclaved “buffer filtration line”, with the outlet positioned directly over the biotainer. The inlet is positioned in the unsterile washing buffer (Grade B side). The buffer is sterile filtered and a filter integrity test is carried out. The filled biotainer with filtered solution is closed with a 2-ported cap, venting filter, hoses, and sterile connector. The mass of the final solution is weighed for the process balance. The container is then labeled and stored at 5±3° C.

TABLE 4
Buffer Preparation
Formulation Table

| | | Component | | Release | | Storage | |
|---|---|---|---|---|---|---|---|
| Formulation | Components | Weights (g/L) | | Criteria | Specification | Conditions | Expiry Date |
| Washing | Potassium | 1-5 | g | Appearance of | Clear, colorless | Room | 24 Hours |
| Buffer | DiHydrogen | | | Solution | and solution free | Temperature | unfiltered. |
| | Phosphate | | | | from particulate | | 6 weeks once |
| | (KH₂PO₄), | | | | matter | | filtered. |
| | DiSodium | 10-20 | g | pH of Solution | 6.8-7.8 | | |
| | Hydrogen | | | | | | |
| | Orthophosphate | | | | | | |
| | (Na₂HPO₄) | | | | | | |
| | Sodium | 100-200 | g | Isotonicity of | 300-380 | | |
| | Chloride (NaCl) | | | Solution | mOsmol/kg | | |
| | Potassium | 1-5 | g | Endotoxin | <0.25 EU/mL | | |
| | Chloride (KCl) | | | Content | | | |
| | Sucrose | 100-500 | g | Sterility | Pass | | |

| | |
|---|---|
| WFI | QS to 5-20 L |

B. Pre-Culture Process

a. Preparation and Pre-Incubation of the Pre-Culture

Under aseptic operating conditions (cleanroom Grade A/B), the Erlenmeyer flasks (pre-sterilized disposable shaking flasks) are filled with the autoclaved pre-culture medium. The pre-culture is split into two steps: pre-culture 1 (PC 1) and pre-culture 2 (PC 2), see Table 5. PC 1 consists of a shaking flask with 50-150 mL medium and PC 2 consists of five shaking flasks with 250-750 mL medium each. Gravimetric measurement has to be used for aliquotation. One of the pre-culture PC 2 flasks is used exclusively for analytical testing during the incubation process. For the OD600 measurement (blank value, dilution of broth) and the adjustment of the photometer a sample of 50±20 mL is taken. Directly before the analytic of the optical density, the photometer is set to the zero value with a medium sample. The prepared shaker flasks are labeled with strain, batch, and flask number, and transferred to the shaker for pre-incubation. This is carried out for 8-36 hours at 30-40° C. and a shaking frequency of 100-200 rpm. A data logger is placed in the incubator to monitor temperatures; the monitoring will be stopped and evaluated at the end of the pre-culture process.

b. Start of Incubation of PC 1

PC 1 is inoculated with 1 vial (500-1000 uL) of the corresponding master cell bank Listeria monocytogenes. The MCB is thawed completely at room temperature and immediately transferred in the shake flask PC 1 using a 1 mL sterile pipette.

c. Incubation of PC 1 and Start of Incubation of PC 2

The incubation is carried out at 30-40° C. and a shaking frequency of 100-200 rpm. A data logger is placed in the incubator to monitor temperatures for pre-incubation, and the incubation of PC1 without any pauses for the pre-culture process. PC 1 is incubated until OD600 of 1-4 is reached. The ending pH should be below 6.5. The first measurement of OD600 and pH takes place after 7±1 h (3±1 mL sample). When the OD600 increases above 0.4, the broth has to be diluted with factor 10 (using sterile medium). If the target OD600 is already achieved, the pre-culture is stopped. Until the target is reached, sampling and testing for both optical density and pH will take place in 30±15 minute intervals. Before inoculation of PC 2, the media aliquots are pre-incubated for 8-36 h to check visually the sterility of the medium; only flasks without turbidity have to be used. Each flask of PC 2 is inoculated with 15±1 mL broth of PC 1. The incubation of PC 2 is continued until an optical density target of 0.5-3 is reached.

d. Incubation of Pre-Culture PC 2

The incubation of PC 2 is continued until OD600 of 0.5-3 is reached. pH should be below 7.0. The first sampling (3±1 mL) of the flask PC 2e for the analytic of OD600 and pH takes place at 2.5±0.5 h after inoculation. Until the target OD600 is not met, the sampling will take place in 30±15 min intervals. When the OD600 increases above 0.4 the broth has to be diluted with factor 10 using sterile medium. After the target is met, the four PC 2 cultures are sampled and analyzed regarding OD600 and pH. The values are double checked and the cultures released for use by the supervisor. Only the released cultures of PC 2 are pooled into the prepared 5 L biotainer. The mass of inoculum is weighed, with a target mass of 1-4 kg. A 5±1 mL sample of the pooled culture is taken immediately after pooling for the determination of the viable cell count (VCC), OD600, and pH. The transfer of the inoculum from the Grade A/B cleanroom to the pilot plant is carried out immediately. During transport, the culture is oxygenated via manual shaking. The time between pooling and the start of the fermentation process is below 20 minutes.

TABLE 5
Preculture Step

| | Operational | | | In Process | Acceptance |
|---|---|---|---|---|---|
| Process Step | Parameters | | | Controls | Criteria |
| Inoculum | Temperature | 30-40° | C. | OD₆₀₀ | 1-4 |
| prep: | Agitation | 100-200 | rpm | pH | ≤6.5 |
| Preculture 1 | | | | | |
| Preculture 2 | Temperature | 30-40° | C. | OD₆₀₀ | 0.5-3 |
| | Agitation | 100-200 | rpm | pH | ≤7.0 |

| | | | | |
|---|---|---|---|---|
| Pool | NA | NA | OD₆₀₀ | 0.5-3 |
| | | | pH | ≤7.0 |
| | | | Viable Cell | Process |
| | | | Count | Trending |

C. Fermentation and Crossflow Process

a. Preparation of Transfer Lines for the SUB and CFF Process

Before the start of the production, different transfer lines are prepared in a Grade D cleanroom. The lines are sterilized and finalized under Grade A/B cleanroom conditions. Non-sterile materials which come into direct contact with the product have to be washed with WFI, assembled, and autoclaved afterwards. The inoculum, feed and harvest line are split in two parts. The SUB-side part is connected under cleanroom Grade A conditions with the bag of the SUB. The other half is added during the process taking place in the Grade D cleanroom. The washing buffer line CFF has already been prepared during media preparation.

b. Installation of the Single-Use-Bioreactor (SUB)

The single use bioreactor (SUB) has to be prepared. Sterile connections are already added under aseptic conditions in step C.a. The bioreactor is built up in the pilot plant (Grade D cleanroom), and the periphery is finalized (aeration lines, base line, sampling line). The aeration filters are autoclaved, and connected by sterile connectors. The connection of the medium filtration line between the sterile filter and medium tank takes place in a Grade D condition in the mobile LAF. The prepared fermentation medium is sterile filtered directly into the CultiBag. The medium filtration line is removed by tube welding after sterile filtration of the complete medium. Subsequently, a filter integrity test is carried out. The sterile filter has to pass this test before the inoculation of the fermentation medium. The setpoints for the pre-incubation of the fermentation medium are changed to the following values: Temperature: 30-40° C., Stirrer: 100-200 rpm, Overlay Aeration: 2-10 lpm, and Sparger Aeration: 0.5-5 lpm (both with air). Recording of data is carried out with the system software, and used in order to achieve the parameter targets from the start of pre-incubation; actual values are recorded. pH and pO¬2 probes are calibrated when the process parameters are stable. Then, a sterile sample of 50±20 mL is taken. The pH must be checked outside the system via external pH meter, and recalibrated if necessary (if a deviation exists between online and offline pH measurements greater than 0.15). The rest of the volume is stored for OD600 analytics for the blank value of diluted broth at room temperature. The external cooling unit must be switched on and set to 2-15° C.

c. Fermentation

Before inoculation, the medium is pre-incubated for 4-24 h. The values during pre-incubation are controlled to check the success of the sterile filtration and the actual data have to be recorded. The temperature must be a constant 30-40° C. A drift of the pH value is most likely due to the aging process of the pH probe and the degassing of the medium, but the value has to be in the range of 6-7. A check of the external pH is made (sampling of 3 mL medium with the sampling line by tube welding). If a higher deviation than 0.15 between offline and online pH exists, a recalibration is necessary. The percentage of dissolved oxygen (pO2) must also be nearly constant during pre-incubation. The level before inoculation is 50-150%. There is an adaptation with higher sparger aeration possible. If the other parameters are in the desired range and no turbidity occurs, the process can continue. pO2 is controlled by sparger aeration with oxygen. Therefore, the aeration is switched from air during pre-incubation to oxygen for fermentation. The adjustment of pH is carried out with NaOH solution. The tubing of the base line is filled and the base consumption rate is reset to zero. Then, the pH and pO2 control is started. The time range from pooling until the start of inoculation has to be below 20 minutes. The inoculum line with the pooled PC 2 is installed by sterile connectors under cleanroom Grade D conditions. The broth is transferred by peristaltic pump. During this process the culture is shaken manually to guarantee oxygen supply. Afterwards, the system is synchronized and the fermentation process begins. During fermentation, at the incubation time of 1-3 h and 4-6 h, a 3±1 mL sample is taken to analyze OD600 and pH. When the OD increases above 0.4 the broth has to be diluted with factor 10 (dilution with sterile medium). The values of the off and online pH values must be compared. This time, if a deviation greater than 0.2 occurs, a recalibration is necessary in order to guarantee the pH target of 7±0.2. The sampling is carried out by hose welding in the sampling manifold. At OD600 of 2.5-4.5, a temperature shift from 30-40° C. down to 20-30° C. is carried out to prevent high foam formation. The fermentation is continued to a culture density of OD600 3.5-6.5. The cooling of the fermentation broth down to 5-10° C. starts. The setpoint of the cooling unit is changed down to 1-5° C. Fermentation is finished when the culture temperature reaches a temperature of ≤20° C. and the concentration process is then initiated. For the concentration process, the pH of the broth will be adjusted to 6.5-7.5 with the connected NaOH base. Directly before concentration, a 5±1 mL sample is tested for OD, pH, and VCC. The mass of the rest of the volume of the base is weighed for the process mass balance. The mass of all samples, dead volumes, and discarded liquids must be estimated for the mass balance of the process.

TABLE 6
Fermentation Step

| | Operational | | | In Process | Acceptance |
|---|---|---|---|---|---|
| Process Step | Parameters | | | Controls | Criteria |
| 10 L SUB | Temperature | 30-40° | C. | OD₆₀₀ | ≥0.5 |
| | Agitation | 100-200 | rpm | pH | 6.5-7.5 |
| | Aeration Sparge | 0-3 | lpm | | |
| | Air Overlay | 5-15 | lpm | | |
| | DissolvepO2 (PO2) | 50-150% | | | |
| | pH | 6.5-7.5 | | | |
| Fermentation- | NA | 20-40° C. ↓ 20-30° C. | | OD₆₀₀ | 2.5-4.5 |
| Temperature | | | | pH | 6.5-7.5 |
| Shift | | | | | |
| Fermentation | Temperature | ≤20° | C. | OD₆₀₀ | 3.5-6.5 |
| Cooling Start | agitation | 100-200 | rpm | pH | Yes/No |
| | dissolved O₂ | 50-150% | | Adjustment | |
| | pH | 6.5-7.5 | | | |
| Fermentation | Temperature | ≤20° | C. | OD₆₀₀ | Process |
| Cooling End | agitation | 100-200 | rpm | pH | Trending |

| | | | |
|---|---|---|---|
| dissolved O₂ | 50-150% | Viable Cell | 6.0-8.0 |
| pH | 6.5-7.5 | Count | ≥1 × 10⁸ |
| | | | CFU/mL |

d. Preparation and Sterilization of the Crossflow Filtration Plant

Three filter cassettes are installed. The success of cleaning and filter integrity testing are documented in the crossflow protocol. The process is recorded by the system software. The prepared CFF lines are connected to the plant before the CFF plant is sterilized less than 72 hours before the downstream process is started.

e. Tangential Flow Filtration/Crossflow Filtration Process

(Concentration/Diafiltration)

Once the bacteria grow to a specific density, the Concentration and Diafiltration section of the assembly (FIGS. 24A and 24C) is used to remove the fermentation media and concentrate the batch by recirculating the mixture of fluid, including the fermentation media, and the construct through a loop including conduit 5, a hollow fiber filter 23, and the retentae bag 2. A 2-fold concentration is carried out, and the circulation may continue until the product reaches its final, 2-fold concentration.

During diafiltration, a wash/formulation buffer bag (e.g., a bag 29 holding wash/formulation buffer) is connected to a coupler 11 the retentae bag 1 of the tangential flow filtration assembly (used for concentration/diafiltration of the fermented media) (FIGS. 24A-C) and the bacterial cells are washed/purified (Diafiltration: ≥8 Diavolumes ≥4 L) while the pump 40 continues to circulate the remaining mixture and the filter 23 continues to remove media from the mixture. The remaining media is replaced with formulation buffer via a cross flow filtration in the hollow fiber filter, and the product is diluted to the final concentration. In some embodiments, the formulation buffer may be added at the same rate that fluid is removed to the permeate bag 2 by the filter 23, such that a substantially constant concentration of the construct is maintained while the old media is replaced with formulation buffer and diafiltration is started after the concentration is reached. The retentae bag 1 may be kept on a scale to measure and maintain a constant volume in the bag during diafiltration.

Before the concentration process, the connections to the cooling unit are opened. The set point of this cooling unit is 0° C. The connection between the SUB and the CFF/TFF system is carried out via sterile connectors. The system software is synchronized with the start of filtration; after complete fermentation, the broth is concentrated two-twentyfold to a mass of 10-1 kg. The setpoints for the pressure control during concentration are Pfeed=0.5-3 bar, Pretentate=Ppermeate=0-3 bar (open valves, pressure fluctuations are process related). The temperature in the retentate decreases at the start of the process, due to the room temperature of the CFF/TFF plant. After 15 minutes, the target of less than 20° C. has to be achieved and must be stable throughout the process. After the concentration step a diafiltration with 5-20 L washing buffer is carried out. The line for the washing buffer, kept cool in the refrigeration room, is connected directly before the concentration process. The setpoints for the pressure control are changed to Pfeed=0.5-3 bar with Pretentate=Ppermeate=0-3 bar. This process must be performed within 4 hours and the washed drug substance has to achieve a weight of 1-10 kg (2-20 fold concentration) in the reservoir tank of the CFF. At the end of the process, the harvest is transferred into a 5 L biotainer. The mass of the washed drug substance is determined by gravimetric measurement during aliquotation. The harvest line is disconnected after the CFF process by tube welding and has to be transferred into production for sampling and aliquotation. Prior to aliquoting to the patient the drug product may be sampled for pH, appearance, osmolality, colony PCR, actA gene presence, SIINFEKL tag (antigen presentation), monosepsis, viable cell count, % live/dead & endotoxin.

TABLE 7
Concentration/Diafiltration

| | Operational | | | In Process | Acceptance |
|---|---|---|---|---|---|
| Processing Step | Parameters | | | Controls | Criteria |
| Start of cooling | Temperature | 0° | C. | NA | NA |
| | Set-point. | | | | |
| Concentration | P_{feed} | 0.5-3 | bar | NA | NA |
| | Pᵣₑₜₑₙₜₐₜₑ | 0-3 | bar | | |
| | Pₚᵣₑₘₑₐₜₑ | 0-3 | bar | | |
| | Conc. | 2-20 | fold | | |
| | Factor | | | | |
| Diafiltration | P_{feed} | 0.5-3 | bar | NA | NA |
| | Pᵣₑₜₑₙₜₐₜₑ | 0-3 | bar | | |
| | Pₚᵣₑₘₑₐₜₑ | 0-3 | bar | | |
| | Diavolume | 5-20 | L | | |

D. Harvest and Aliquotation of Drug Substance

The biotainer with the drug substance is closed completely and transferred for sampling and aliquotation in a Grade A/B cleanroom. The biotainer may include the bags shown attached to the manifold 39 of the assembly shown in FIGS. 25-26. In such embodiments, the connection may be made by the fully enclosed system such that no additional sterilization is required. The biotainer with the harvest is weighed before aliquotation. Afterwards, a sample of 5±1 mL is taken to analyze OD600, pH, and VCC. Due to dilution factor of 100 (two factor 10 dilutions), the preparation for the measurements of OD600 is performed three times to get a representative average value. Each dilution step is vortexed for 5±2 sec and directly processed to avoid sedimentation. The target OD600 is ≥15-45. The harvest is split under cleanroom class A conditions in ≥10 pieces of 50-150 mL aliquots i biotainers for the fill and finish process.

The aliquots are stored in two different freezers at −80±10° C. The completely drained biotainer is weighed again to determine the mass of produced drug substance (target: 1-10 kg)

TABLE 8
Harvest/Dispensing

| Processing | Operational | | | In Process | Acceptance |
|---|---|---|---|---|---|
| Step | Parameters | | | Controls | Criteria |
| Harvest/ | Dispensing | 100 ± 5 | mL | OD₆₀₀ | ≥50 |
| Dispensing | weight | | | pH | 7.2 ± 0.3 |
| | Storage | −80 ± 10° | C. | Viable Cell | 1 × 10⁸- |
| | temp | | | Count | 1 × 10¹¹ |
| | | | | | CFU/mL |

E. Aseptic Fill into Vials

The viable cell count of one aliquot of drug substance is determined 2-7 days before filling in order to adjust the cell concentration for filling. The manufacturing starts with thawing of the drug substance aliquots (Biotainer with 50-150 mL of drug substance) at 2° C. to 8° C. overnight. Under aseptic conditions subsequent to thawing, the drug substance is adjusted to 1×10⁸-1×10¹¹ CFU/mL with washing buffer (product bulk) and filled to a volume of 1-100 mL in the appropriate sized vials at room temperature. The vials are capped, labeled, packed, and stored at −80±10° C.

While certain features of the inventions have been illustrated and described herein, many modifications, substitutions, changes, and equivalents will now occur to those of ordinary skill in the art. It is, therefore, to be understood that the appended claims are intended to cover all such modifications and changes as fall within the true spirit of the disclosure.

1. A process for the manufacturing of a formulation comprising a drug substance, said drug substance comprising a recombinant Listeria strain, said recombinant Listeria strain comprising a nucleic acid comprising an open reading frame encoding a recombinant polypeptide, said recombinant polypeptide comprising a prostate specific antigen (PSA) or a chimeric HER2 (cHER2) antigen fused to a Listeriolysin O (LLO) polypeptide, the method comprising the steps of:
a) Aseptically preparing a first pre-culture media (PC 1) in a container and a second pre-culture media (PC2) in at least two containers.
b) Aseptically adding a working cell bank (WCB) comprising said recombinant Listeria into PC 1.
c) Aseptically inoculating each container of said PC2 with an aliquot from said PC 1.
d) Incubating each container in c) until a target optical density (OD) is reached and pooling culture media from each of said container into a larger biotainer.
e) Preparing fermentation media and adding said fermentation media into a fermenter system.
f) Inoculating said fermentation media with the pooled culture media from step d) and initiating a fermentation process until a target optical density (OD) is reached.
g) Aseptically connecting said fermenter system to a filtration system and concentrating said drug substance within said fermentation media to a desired weight.
h) Obtaining a retentate or harvest solution comprising said drug substance from step g) and exchanging the spent fermentation media with an appropriate formulation for human use.
i) Aseptically transferring said harvest comprising said drug substance into biotainers.
j) Aseptically aliquoting said drug substance into vials for clinical use. Determining a viable cell count (VCC) prior to aliquoting into vials.
k) Disinfecting, inspecting, labeling, packaging and distributing said vials to clinical sites.

2. The process of claim 1, wherein said PC1 and PC2 are aseptically sampled and tested for Optical Density at 600 nm (OD_{600 nm}), and pH at regular intervals until said target OD is reached.

3. The process of claim 1, wherein said PC1 and PC2 is incubated for 12-24 h to ensure sterility.

4. The process of claim 1, wherein said fermentation media is pre-incubated for 12±6 h to verify sterility prior to inoculation with said working cell bank.

5. The process of claim 1, wherein the pooled culture in d) is sampled to determine the viable cell count (VCC), OD, and pH.

6. The process of claim 1, wherein said initiation of said fermentation process is preceded by a pre-incubation step of the fermentation media.

7. The process of claim 6, wherein said pre-incubation step comprises regulating and maintaining a constant temperature, constant pH, and constant dissolved oxygen percentage (pO₂).

8. The process of claim 7, wherein said pO2 level is controlled by sparger aeration with oxygen.

9. The process of claim 7, wherein said pH of said fermentation process is controlled using an alkylating agent.

10. The process of claim 9, wherein said alkylating agent is NaOH.

11. The process of claim 1, wherein said fermentation process is stopped by cooling the fermentation media to a temperature of ≤20° C. after said target OD has been reached.

12. The process of claim 1, wherein said fermented media is aseptically sampled and tested for OD, pH and viable cell count (VCC) prior to connecting to said filtration system.

13. The process of claim 1, wherein said concentrating step is carried out at a low temperature.

14. The process of claim 13, wherein said low temperature is 0-20° C.

15. The process of claim 1, wherein said fermentation media or broth is concentrated 2-20 fold to a mass of about 1-10 kg.

16. The process of claim 1, wherein said filtration system is a Cross Flow Filtration (CFF) or Tangential Filtration (TFF) system.

17. The process of claim 16, wherein said fermenter system is aseptically connected to the inlet of said TFF system.

18. The process of claim 1, wherein said drug substance is concentrated 2-20 fold.

19. The process of claim 1, wherein said exchanging comprises diafiltering said harvest comprising said drug substance with washing buffer.

20. The process of claim 1, wherein said harvest comprising said DS is transferred into a biotainer for sampling and aliquoting.

21. The process of claim 20, wherein said biotainer is a 1-10 L biotainer.

22. The process of claim 21, wherein said harvest is sampled and tested for OD₆₀₀, pH, and viability cell count (VCC) prior to aliquoting into one or more biotainers of smaller volume.

23. The process of claim 22, wherein said one or more biotainers comprise a volume of 125 ml.

24. The process of claim 22, wherein said one or more biotainers are stored at −80° C.±10° C. until they are aseptically aliquoted into vials for clinical use.

25. The process of claim 24, wherein a VCC is determined prior to aliquoting into vials.

26. The process of claim 25, wherein said VCC is determined 2-7 days prior to aliquoting in to said vials.

27. The process of claim 25, wherein determination of said VCC is used for the calculation of a dilution factor and required amount for formulation of the DS with the same buffer used for the diafiltration step in step h).

28. The process of claim 24, wherein said aliquots are adjusted to 1×10⁸-1×10¹¹ CFU/mL with a formulation buffer solution and filled to a desired volume in vials.

29. The process of claim 24, wherein said desired volume is about 1-5 ml.

30. The process of claim 24, wherein said vials are stored at ≤−80±10° C. and thawed at room temperature prior to any further processing.

31. The process of claim 1, wherein said LLO is an N-terminal LLO.

32. The process of claim 31, wherein said N-terminal LLO comprises SEQ ID NO: 2.

33. The process of claim 1, wherein said PSA comprises SEQ ID NO: 5.

34. The process of claim 1, wherein said recombinant polypeptide comprises SEQ ID NO: 13.

35. The process of claim 1, wherein said cHER2 comprises SEQ ID NO: 15.

36. The process of claim 1, wherein said recombinant polypeptide comprises SEQ ID NO: 17.

37. The process of claim 1, wherein said recombinant Listeria comprises a mutation, deletion or inactivation of an endogenous dal, dat and actA gene.

38. The process of claim 1, wherein said nucleic acid molecule is in a plasmid in said recombinant Listeria strain.

39. The process of claim 38, wherein said plasmid is stably maintained in said recombinant Listeria strain in the absence of antibiotic selection.

40. The process of claim 38, wherein said plasmid does not confer antibiotic resistance upon said recombinant Listeria.

41. The process of claim 38, wherein said plasmid comprises an open reading frame encoding a metabolic enzyme that complements said dal/dat gene mutation, deletion or inactivation.

42. The process of claim 41, wherein said metabolic enzyme encodes a D-alanine racemase enzyme or a D-amino acid transferase enzyme.

43. The process of claim 1, wherein said recombinant polypeptide is expressed by said recombinant Listeria.

44. The process of claim 1, wherein said Listeria has been passaged through an animal host.

45. The process of claim 1, wherein said recombinant Listeria is a Listeria monocytogenes.

46. A tangential flow filtration device comprising:
a retentate bag, the retentate bag comprising:
a recirculation outlet;
a recirculation inlet; and
a diafiltration inlet;
a permeate bag;
a filter; and
a circulation pump;
wherein a first conduit defines a first fluid path from the recirculation outlet to the recirculation inlet, and wherein the first conduit fluidly connects the retentate bag, the circulation pump, and the filter, such that the circulation pump is configured to pump a mixture from the retentate bag to the filter and back to the retentate bag;
wherein a second conduit defines a second fluid path from the filter to the permeate bag, wherein the filter is configured to allow at least a portion of the mixture into the permeate bag; and
wherein the recirculation outlet is defined proximate the retentate outlet, such that the retentate outlet is configured to mix the mixture of the retentate bag proximate the retentate outlet.

47. The device of claim 46, further comprising a valve on the first conduit, wherein the valve is configured to selectively control a pressure in the first conduit.

48. The device of claim 47, wherein the pressure is 3 psi.

49. The device of claim 46, wherein at least one of the recirculation outlet, recirculation inlet, or diafiltration inlet is disposed at or proximate a bottom of the retentate bag in an operational position.

50. The device of claim 49, wherein the recirculation outlet and the diafiltration inlet are disposed at or proximate the bottom of the retentate bag.

51. The device of claim 46, further comprising at least one optical density sensor configured to detect an optical density of the mixture.

52. The device of claim 51, wherein the at least one optical density sensor is optically connected to the retentate bag.

53. The device of claim 51, wherein the at least one optical density sensor is optically connected to the permeate bag.

54. The device of claim 51, wherein the at least one optical density sensor is optically connected to the first conduit.

55. The device of claim 46, further comprising at least one pressure sensor coupled to the first conduit.

56. A method of manufacturing a construct, the method comprising:
providing a retentate bag having a mixture of a first fluid and a construct;
concentrating the construct by:
circulating the mixture to a filter,
wherein the filter is fluidly connected to a permeate bag, such that the filter is configured to direct at least a portion of the first fluid passing through the membrane to enter the permeate bag and allow a remaining portion of the mixture to return to the retentate bag,
diafiltering by:
adding a second fluid to the remaining portion of the mixture to form a second mixture; and
circulating the second mixture to the filter;
wherein at least the second mixture is circulated at a flow rate,
wherein the flow rate causes an at least partially turbulent flow of the second mixture, and
wherein the flow rate is defined where little or no shearing the construct occurs.

57. The method of claim 56, wherein the construct is concentrated 2-fold.

58. The method of claim 56, wherein the flow rate is from 0.450 L/min to 0.850 L/min.

59. The method of claim 58, wherein the flow rate is 0.650 L/min.

60. The method of claim 56, further comprising maintaining a predetermined pressure at the filter.

61. The method of claim 60, wherein the predetermined pressure is maintained by controlling a valve to constrict the flow of the first mixture or the second mixture.

62. The method of claim 56, wherein the at least partially turbulent flow is detected with pressure sensors positioned before and after the filter in a fluid conduit.

63. The method of claim 62, wherein the pressure sensors are configured to detect a high pressure differential indicating a biofilm formation.

64. The method of claim 63, further comprising increasing the flow rate in response to a high pressure differential.

65. The method of claim 56, wherein the shearing is detected with one or more optical density sensors.

66. The method of claim 65, wherein the one or more optical density sensors detect a change in the optical density of the first mixture or the second mixture.

67. The method of claim 65, wherein the one or more optical density sensors are disposed in the permeate bag.

68. The method of claim 65, wherein the change is detected in comparison a baseline optical density.

69. The method of claim 56, further comprising a flow controller electrically connected to the circulation pump and configured to control the flow rate.

70. The method of claim 56 further comprising at least one flow rate sensor, wherein the at least one flow rate sensor comprises a first pressure sensor disposed upstream of the filter and a second pressure sensor disposed downstream of the filter, and wherein the minimum threshold is defined when a difference between a first pressure detected by the first pressure sensor and a second pressure detected by the second pressure sensor reaches a predetermined threshold.
